(19) United States
(12) Patent Application Publication
(10) Pub. No.: US 2007/0232577 A1
(43) Pub. Date: Oct. 4, 2007

(54) Synthetic pentacyclic triterpenoids and derivatives of betulinic acid and betulin

(75) Inventors: Ze-Qi Xu, Woodridge, IL (US); Ali Koohang, Plainfield, IL (US); Aye Aye Mar, Tinley Park, IL (US); Nathan D. Majewski, Bryn Mawr, PA (US); David A. Eiznhamer, Bloomingdale, IL (US); Michael T. Flavin, Darien, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Advanced Life Sciences, Inc., Woodridge, IL (US)

(21) Appl. No.: 11/728,284

(22) Filed: Mar. 23, 2007

Related U.S. Application Data
(60) Provisional application No. 60/785,309, filed on Mar. 23, 2006.

(51) Int. Cl. A61K 31/58; A61K 31/56; C07J 53/00

(52) U.S. Cl. 514/169; 514/176; 540/47; 552/511

(57) ABSTRACT

The present invention comprises small molecule inhibitors of cell proliferative conditions, in particular cancer and conditions associated with cancer. For example, associated malignancies include ovarian cancer, cervical cancer, breast cancer, colorectal cancer, and glioblastomas, among others. Accordingly, the compounds of the present invention are useful for treating, preventing, and/or inhibiting these diseases. Thus, the present invention also comprising pharmaceutical formulations comprising the compounds and methods of using the compounds and formulations to inhibit cancer and treat, prevent, or inhibit the foregoing diseases.

### CROSS-REFERENCE TO OTHER APPLICATIONS

This application claims the benefit of U.S. Provisional Application No. 60/785,309 filed Mar. 23, 2006, which is incorporated by reference in its entirety.

### FIELD OF THE INVENTION

This invention relates to the field of inhibitors of cell proliferative conditions. In particular, the invention relates to inhibitors of cancer and conditions associated with cancer.

### BACKGROUND OF THE INVENTION

Betulinic acid (3β-hydroxy-lup-20(29)-en-28-oic acid, also known as (1R,3aS,5aR,5bR,7aR,9S,11aR, 11bR,13aR,13bR)-9-hydroxy-5a,5b,8,8,11a-pentamethyl-1-(prop-1-en-2-yl)icosahydro-1H-cyclopenta[a]chrysene-3a-carboxylic acid) (1) and betulin (3β-lup-20(29)-en-3,28-diol or (1R,3aS,5aR,5bR,7aR,9S,11aR,11bR,13aR,13bR)-3a-(hydroxymethyl)-5a,5b,8,8,11a-pentamethyl-1-(prop-1-en-2-yl)icosahydro-1H-cyclopenta[a]chrysen-9-ol) (2) are lupane type triterpenoid molecules which can be isolated from a wide range of plant sources. The birch tree (Betula spp., Betulaceae) is one of the most substantial sources for both molecules.¹ Furthermore, betulin (2) can be converted to betulinic acid (2) in two steps by oxidation with Jones' reagent and selective reduction of the formed betulonic acid (3)² (also known as (1R,3aS,5aR,5bR,7aR,11aR,11bR,13aR,13bR)-5a,5b,8,8,11a-pentamethyl-9-oxo-1-(prop-1-en-2-yl)icosahydro-1H-cyclopenta[a]chrysene-3a-carboxylic acid).

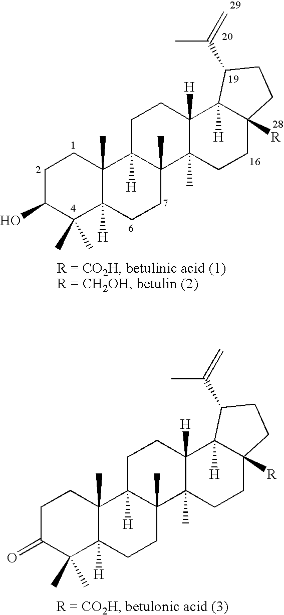

Betulinic acid and betulin have been reported to posses a wide range of biological properties including activities against cancer cell lines, viruses, bacteria and malaria, as well as inflammatory process in general.^{3,4,5} One of the most distinguishing features of betulinic acid is the lack of cytotoxicity against normal and healthy cells such as normal human astrocytes, human melanocytes, normal derma fibroblast, and peripheral blood lymphoblasts.^{5c,6,7}

The molecular mechanism of betulinic acid towards cancer cells is still a subject to continuous investigations and specific target(s) has yet to be identified. However, betulinic acid has been reported as a selective and dose-dependent apoptosis-inducing agent.⁸ Betulinic acid may target the mitochondria directly, thus triggering activation of pro-apoptotic proteins involved in internucleosomal DNA fragmentation, which is independent of both p53 and CD95.⁹ When combined with radiation therapy or with other chemotherapeutic agents, betulinic acid has demonstrated synergistic effects in the in vitro and in vivo systems.¹⁰

In the past few years, there has been a great deal of interest in the synthesis and evaluation of new derivatives of betulinic acid (1) and betulin (2) for their biological activities.¹² Thus, an object of this invention is the identification of betulinic acid and betulin derivative and analog compounds that specifically treat, prevent, inhibit, regulate and/or modulate cancer. In our continuous efforts in searching for molecules effective against cancers with novel mechanism of action, we have embarked upon the design, synthesis and evaluation of triterpenoid derivatives, especially derivatives of betulinic acid (1) and betulin (2).

### SUMMARY OF THE INVENTION

The invention provides compounds, and methods and pharmaceutical compositions comprising the compounds useful for treating diseases such as cancer. In one aspect, the invention provides compounds of the formula 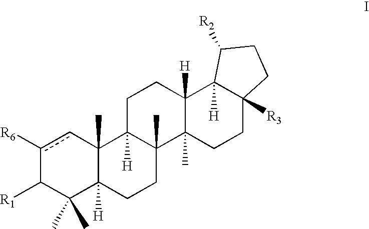
or a pharmaceutically acceptable salt thereof, wherein [symbol] is a single or double bond;

R₁ is H, halo, NH₂, OH, SH, ═O, ═S, ═N—OH, NHR₄, NH(CH₂)R₄, NR₄R₅, OR₄, OCOR₄, OC(O)OR₄, OC(O)NR₄R₅, SR₄, SCOR₄, SC(O)NR₄R₅, SC(O)NR₄R₅, NHCOR₄, NHC(O)OR₄, N(R₅)C(O)OR₄, NHC(O)NR₄R₅, N(R₅)C(O)NR₄R₅, ═N—OR₄, ═N—OCOR₄, OCO(HC═CH)ₙR₄, OCO(CH₂)ₙX, OSO₂(CH₂)ₙX, OSi(R₄)ₙ(R₅)₃₋ₙ, or SCO(CH₂)ₙX;

R₂ is C(CH₃)₂ or C(═CH₂)CH₃;

R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)ₙR₄, CH₂OCO(CH₂)ₙX, CH₂NH₂, CH₂NHR₄, CH₂N(CH₂)ₙR₄, CH₂NR₄R₅, CO₂R₄, C(O)NHR₄, or C(O)NR₄R₅;

R₄ and R₅ are independently H, C(O)X, halo, C₁₋₈ alkyl, aryl-C₁₋₈ alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;

R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;

R₆ is H, halo, Se-aryl, OR₄, CN, CHO, CO₂R₄, or C(R₄)ₙ(R₅)₃₋ₙ, or R₆ together with the ring to which it is attached form

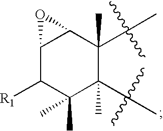

X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and

n=1-5;

provided that when R₁ is oxo, [symbol] is a double bond, R₂ is C(CH₃)₂ and R₃ is CO₂H, R₆ cannot be CN, Cl or CHO; and when R₁ is oxo, [symbol] is a double bond, R₂ is C(CH₃)₂ and R₃ is CO₂Me, R₆ cannot be CN, OMe or CHO.

In one embodiment according to formula I, R₁ is OSi(CH₃)₂-tert-butyl, OSi(CH₃)₃, OH, ═O, O—C(O)—CH₃, OCO(CH₂)OCH₃, OCO(HC═CH)phenyl wherein the phenyl is substituted with two OCOCH₃ or OH, NH(CH₂)₂—OH, NH(CH₂)₂—Cl, NH(CH₂)₂—SH, NH(CH₂)-phenyl or NH(CH₂)₂-phenyl wherein the phenyl is substituted with OH, NH₂, O-pyranyl, NH(CH₂)₂—NHC(O)O-tert-butyl or NH(CH₂)benzodioxolyl.

In another embodiment according to formula I, R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

In yet another embodiment, the invention provides compounds of the formula 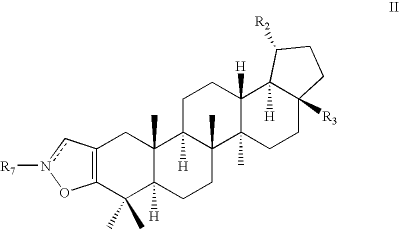
or a pharmaceutically acceptable salt thereof, wherein

[symbol] is a single or double bond, provided that when [symbol] is a double bond, R₇ is absent;

R₂ is C(CH₃)₂ or C(═CH₂)CH₃;

R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)ₙR₄, CH₂OCO(CH₂)ₙX, CH₂NH₂, CH₂NHR₄, CH₂N(CH₂)ₙR₄R₅, CH₂NR₄R₅, CO₂R₄, C(O)NHR₄, or C(O)NR₄R₅;

R₄ and R₅ are independently H, C(O)X, halo, C₁₋₈ alkyl, aryl-C₁₋₈ alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;

R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;

R₇is OH;

X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and

n=1-5.

In one embodiment according to formula III, R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

In still another embodiment, the invention provides compounds of the formula 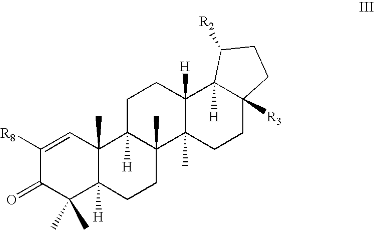
or a pharmaceutically acceptable salt thereof, wherein

R₂ is C(CH₃)₂ or C(═CH₂)CH₃;

R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)ₙR₄, CH₂OCO(CH₂)ₙX, CH₂NH₂, CH₂NHR₄, CH₂N(CH₂)ₙR₄R₅, CH₂NR₄R₅, CO₂R₄, C(O)NHR₄, or C(O)NR₄R₅;

R₄ and R₅ are independently H, C(O)X, halo, C₁₋₈ alkyl, aryl-C₁₋₈ alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;

R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;

R₈ is H, CN, halo, Se-phenyl, OC₁₋₈ alkyl or C(O)H, or R₈ together with the ring to which it is attached form

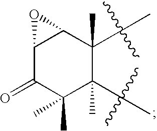

X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and

n=1-5;

provided that when R₂ is C(CH₃)₂ and R₃ is CO₂H, R₈ cannot be CN, Cl or CHO; and when R₂ is C(CH₃)₂ and R₃ is CO₂Me, R₈ cannot be CN, OMe or CHO.

In one embodiment according to formula III, R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

In another embodiment according to formula III, R₈ is H, CN, CHO, Cl or OCH₃.

In another aspect, the invention provides pharmaceutical compositions comprising a compound according to any one of formula I-III and pharmaceutically acceptable carrier, excipient, or diluent.

In yet another aspect, the invention provides methods for inhibiting cancer in a cell comprising contacting the cell in which inhibition is desired with an effective amount of a compound according to any one of formula I-III or a pharmaceutical composition comprising a compound according to any one of formula I-III and pharmaceutically acceptable carrier, excipient, or diluent.

In still another aspect, the invention provides methods of treating a disease comprising administering to a patient a pharmaceutical composition comprising a compound according to any one of formula I-III and pharmaceutically acceptable carrier, excipient, or diluent. In one embodiment, the disease involves a cell proliferative condition. In another embodiment, the cell proliferative condition is cancer. In still another embodiment, the cancer is melanoma, glioblastoma, ovarian carcinoma, colon carcinoma, and breast carcinoma, or cervical cancer.

In another embodiment, the invention provides methods for inhibiting viruses, bacteria or malaria in a cell comprising contacting the cell in which inhibition is desired with an effective amount of a compound according to any one of formulas I-III or a pharmaceutical composition comprising a compound according to any one of formula I-III and pharmaceutically acceptable carrier, excipient, or diluent. In still another embodiment, the invention provides methods for treating inflammation comprising administering to a patient a pharmaceutical composition comprising a compound according to any one of formula I-III and pharmaceutically acceptable carrier, excipient, or diluent.

### DETAILED DESCRIPTION OF THE INVENTION

The chemistries described above represent methods for the syntheses of compounds of the general structural formula I-III and, therefore, the present invention relates to compounds, compositions and methods for the prevention and inhibition of tumor growth and for the treatment of malignant tumors such as melanoma, glioblastoma, ovarian carcinoma, colon carcinoma, and breast carcinoma.

Definitions

As used in the present specification, the following words and phrases are generally intended to have the meanings as set forth below, except to the extent that the context in which they are used indicates otherwise or they are expressly defined to mean something different.

The symbol “−” means a single bond, “=” means a double bond, “−” means a triple bond, “ [symbol] ” means a single or double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

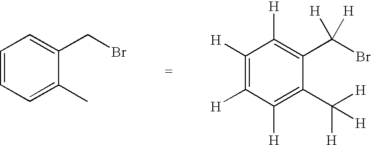

“Alkyl” is intended to include linear, branched, or cyclic hydrocarbon structures and combinations thereof, inclusively. For example, “C₆ alkyl” may refer to an n-hexyl, iso-hexyl, cyclobutylethyl, and the like. Lower alkyl refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more that eight carbon atoms. Exemplary alkyl groups are those of C₂₀ or below. Cycloalkyl is a subset of alkyl and includes cyclic hydrocarbon groups of from three to thirteen carbon atoms. Examples of cycloalkyl groups include c-propyl, c-butyl, c-pentyl, norbornyl, adamantyl and the like. In this application, alkyl refers to alkanyl, alkenyl, and alkynyl residues (and combinations thereof); it is intended to include cyclohexylmethyl, vinyl, allyl, isoprenyl, and the like. Thus when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-ynyl groups; and for example, “propyl” or “C₃ alkyl” each include n-propyl, propenyl, and isopropyl. Alkyl also includes unsaturated hydrocarbon groups, such as alkenyl and alkynyl groups.

“Alkoxy” or “alkoxyl” refers to the group —O-alkyl, for example including from one to eight carbon atoms of a straight, branched, cyclic configuration, unsaturated chains, and combinations thereof attached to the parent structure through an oxygen atom. Examples include methoxy, ethoxy, propoxy, isopropoxy, cyclopropyloxy, cyclohexyloxy and the like. Lower-alkoxy refers to groups containing one to six carbons.

“Aryl” refers to aromatic six- to fourteen-membered carbocyclic ring, and includes mono-, bicyclic or polycyclic groups, for example, benzene, naphthalene, acenaphthylene, anthracene, indane, tetralin, fluorene and the like. Aryl as substituents includes univalent or polyvalent substituents. As univalent substituents, the aforementioned ring examples are named, phenyl, naphthyl, acenaphthyl, anthracenyl, indanyl, tetralinyl, and fluorenyl.

When a group is referred to as “aryl-C₁-C₈ alkyl” or “C₁-C₈-alkyl-aryl”, an aryl moiety is attached to a parent structure via an alkylene group. Examples include benzyl, phenethyl, and the like. Both the aryl and the corresponding alkylene portion of an “C₁-C₆ alkyl-aryl” group may be optionally substituted.

In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Fused-polycyclic” or “fused ring system” refers to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic.

“Halogen” or “halo” refers to fluorine, chlorine, bromine or iodine. “Haloalkyl” and “haloaryl” refer generically to alkyl and aryl groups that are substituted with one or more halogens, respectively. Thus, “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is within the scope of dihaloaryl. The phrase “mono- to per-halogenated” when combined with another group refers to groups wherein one hydrogen, more than one hydrogen, or all hydrogens are replaced with a halo. For example, a “mono- to per-halogenated methyl” would encompass groups such as —CH₂F, —CHCl₂ or —CF₃.

“Heterocycle” or “heterocyclyl” refers to a stable three- to fifteen-membered ring substituent that consists of carbon atoms and from one to five heteroatoms selected from the group consisting of nitrogen, phosphorus, oxygen and sulfur. A heterocycle includes an aromatic heterocyclyl group. For purposes of this invention, the heterocyclyl substituent may be a monocyclic, bicyclic or tricyclic ring system, which may include fused or bridged ring systems as well as spirocyclic systems; and the nitrogen, phosphorus, carbon or sulfur atoms in the heterocyclyl group may be optionally oxidized to various oxidation states. In a specific example, the group —S(O)₀₋₂—, refers to —S— (sulfide), —S(O)— (sulfoxide), and —SO₂— (sulfone). For convenience, nitrogens, particularly but not exclusively, those defined as annular aromatic nitrogens, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms may be optionally quaternized; and the ring substituent may be partially or fully saturated or aromatic. Examples of heterocyclyl groups include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, dioxaphospholanyl, and oxadiazolyl.

Preferred heterocyclyls include, but are not limited to, acridinyl, azocinyl, benzimidazolyl, benzofuranyl, benzothiophenyl, benzoxazolyl, benzthiazolyl, benztriazolyl, pyridotriazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,3,4-triazolyl, and xanthenyl.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted aryl-C₁₋₈ alkyl,” both the “C₁₋₈ alkyl” portion and the “aryl” portion of the molecule may or may not be substituted. A list of exemplary optional substitutions is presented below in the definition of “substituted.”

“Substituted” alkyl, aryl, and heterocyclyl, refer respectively to alkyl, aryl, and heterocyclyl, one or more (for example up to about five, in another example, up to about three) hydrogen atoms are replaced by a substituent independently selected from: alkyl (for example, fluoromethyl), aryl (for example, 4-hydroxyphenyl), arylalkyl (for example, 1-phenyl-ethyl), heterocyclylalkyl (for example, 1-pyridin-3-yl-ethyl), heterocyclyl (for example, 5-chloro-pyridin-3-yl or 1-methyl-piperidin-4-yl), alkoxy, alkylenedioxy (for example methylenedioxy), amino (for example, alkylamino and dialkylamino), amidino, aryloxy (for example, phenoxy), arylalkyloxy (for example, benzyloxy), carboxy (—CO₂H), carboalkoxy (that is, acyloxy or —OC(═O)R), carboxyalkyl (that is, esters or —CO₂R), carboxamido, benzyloxycarbonylamino (CBZ-amino), cyano, acyl, halogen, hydroxy, nitro, sulfanyl, sulfinyl, sulfonyl, thiol, halogen, hydroxy, oxo, carbamyl, acylamino, and sulfonamido. And each substituent of a substituted group is optionally substituted, but these optional substituents themselves are not further substituted. Thus, an optionally substituted moiety is one that may or may not have one or more substituents, and each of the substituents may or may not have one or more substituents. But, the substituents of the substituents may not be substituted.

Some of the compounds of the invention may have imino, amino, oxo or hydroxy substituents off aromatic heterocyclyl systems. For purposes of this disclosure, it is understood that such imino, amino, oxo or hydroxy substituents may exist in their corresponding tautomeric form, i.e., amino, imino, hydroxy or oxo, respectively.

Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as any and all possible stereoisomers, geometric isomers, enantiomers, diastereomers and anomers. All such single stereoisomers, racemates and mixtures thereof, and geometric isomers are intended to be within the scope of this invention.

The description of the invention herein should be construed in congruity with the laws and principals of chemical bonding. It is assumed that when considering generic descriptions of compounds of the invention for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

When a particular group with its bonding structure is denoted as being bonded to two partners; that is, a divalent group, for example, —OCH₂—, then it is understood that either of the two partners may be bound to the particular group at one end, and the other partner is necessarily bound to the other end of the particular group, unless stated explicitly otherwise. Stated another way, divalent groups are not to be construed as limited to the depicted orientation, for example “—OCH₂—” is meant to mean not only “—OCH₂—” as drawn, but also “—CH₂O—.”

In addition to the preferred embodiments recited hereinabove, also preferred are embodiments comprising combinations of preferred embodiments.

Methods for the preparation and/or separation and isolation of single stereoisomers from racemic mixtures or non-racemic mixtures of stereoisomers are well known in the art. For example, optically active (R)— and (S)— isomers may be prepared using chiral synthons or chiral reagents, or resolved using conventional techniques. Enantiomers (R— and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinorna, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

“Pharmaceutically acceptable salt” include acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977;66:1-19 which is incorporated herein by reference.)

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

“Treating” or “treatment” as used herein covers the treatment of a disease-state in a human, which disease-state is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

General Administration

In the second aspect, the invention provides pharmaceutical compositions comprising compounds according to the invention and a pharmaceutically acceptable carrier, excipient, or diluent. In certain other preferred embodiments, administration may preferably be by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, rectally, or via urethral, ocular intratumoral and irrigation method, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, etc. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc. The dosage form can be designed as a sustained release or timed release.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), dextrose, mannitol, polyvinylpyrrolidone, gelatin, hydroxycellulose, acacia, suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants. The liquid formulation can be buffered, isotonic solution.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

In one embodiment, represenative compounds of the invention are illustrated in Table 1. The compounds of Table 1 serve merely to further illustrate the compounds of the invention and do not limit in any way the scope of the invention.

TABLE 1

| Cpd. | | |
|---|---|---|
| No. | Structure | Name |
| 4 | 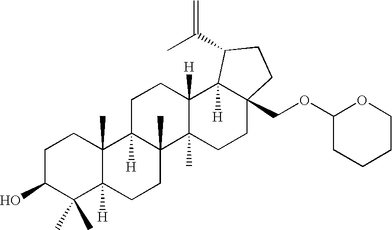 | (1R,3aS,5aR,5bR,7aR,9S,11aR,11bR,13aR,13bR)- 5a,5b,8,8,11a-pentamethyl-1-(prop-1-en-2-yl)- 3a-((tetrahydro-2H-pyran-2-yloxy)methyl)icosa- hydro-1H-cyclopenta[a]chrysen-9-ol |
| 5 | 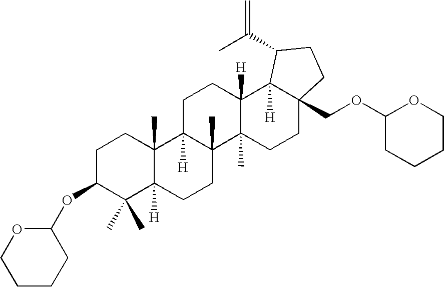 | 2- ((1R,3aS,5aR,5bR,7aR,9S,11aR,11bR,13aR,13b R)-5a,5b,8,8,11a-pentamethyl-1-(prop-1-en-2- yl)-3a-((tetrahydro-2H-pyran-2- yloxy)methyl)icosahydro-1H- cyclopenta[a]chrysen-9-yloxy)tetrahydro-2H- pyran |
| 6 | 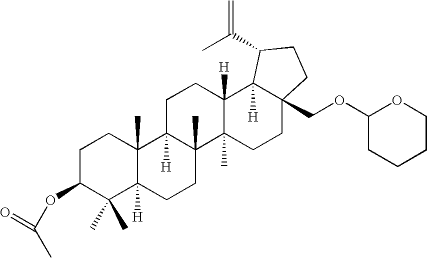 | (1R,3aS,5aR,5bR,7aR,9S,11aR,11bR,13aR,13b R)-5a,5b,8,8,11a-pentamethyl-1-(prop-1-en-2-yl)- 3a-((tetrahydro-2H-pyran-2- yloxy)methyl)icosahydro-1H- cyclopenta[a]chrysen-9-ylacetate |
| 7 | 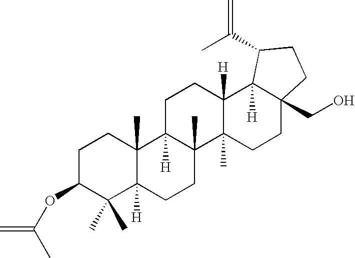 | ((1R,3aS,5aR,5bR,7aR,9S,11aR,11bR,13aR,13b R)-5a,5b,8,8,11a-pentamethyl-1-(prop-1-en-2- yl)-9-(prop-1-en-2-yloxy)icosahydro-1H- cyclopenta[a]chrysen-3a-yl)methanol |
| 8 | 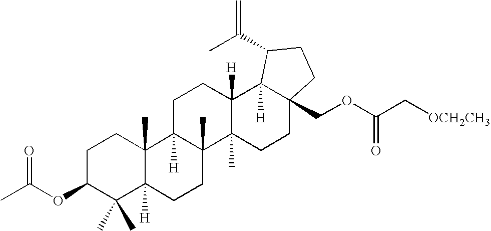 | ((1R,3aS,5aR,5bR,7aR,9S,11aR,11bR,13aR,13b R)-9-acetoxy-5a,5b,8,8,11a-pentamethyl-1-(prop- 1-en-2-yl)icosahydro-1H-cyclopenta[a]chrysen- 3a-yl)methyl 2-ethoxyacetate |
| 9 | 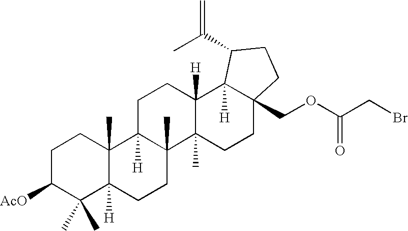 | ((1R,3aS,5aR,5bR,7aR,9S,11aR,11bR,13aR,13b R)-9-acetoxy-5a,5b,8 ,8,11a-pentamethyl-1-(prop- 1-en-2-yl)icosahydro-1H-cyclopenta[a]chrysen- 3a-yl)methyl 2-bromoacetate |
| 10 | 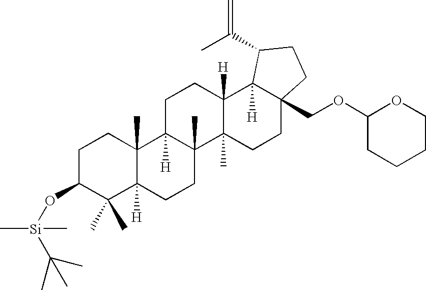 | ((1R,3aS,5aR,5bR,7aR,9S,11aR,11bR,13aR,13b R)-icosahydro-5a,5b,8,8,11a-pentamethyl-1- (prop-1-en-2-yl)-3a-((tetrahydro-2H-pyran-2- yloxy)methyl)-1H-cyclopenta[a]chrysen-9- yloxy)(tert-butyl)dimethylsilane |
| 10a | 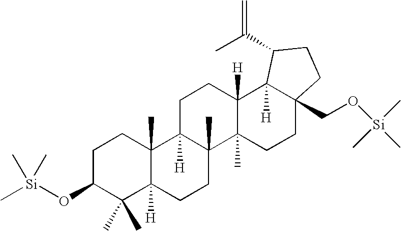 | trimethyl((1R,3aS,5aR,5bR,7aR,9S,11aR,11bR, 13aR,13bR)-5a,5b,8,8,11a-pentamethyl-1-(prop-1- en-2-yl)-3a- ((trimethylsilyloxy)methyl)icosahydro-1H- cyclopenta[a]chrysen-9-yloxy)silane |
| 11 | 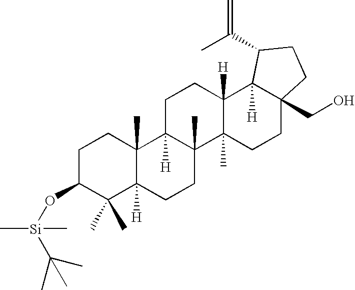 | ((1R,3aS,5aR,5bR,7aR,9S,11aR,11bR,13aR,13b R)-9-(tert-butyldimethylsilyloxy)-5a,5b,8,8,11a- pentamethyl-1-(prop-1-en-2-yl)icosahydro-1H- cyclopenta[a]chrysen-3a-yl)methanol |
| 12 | 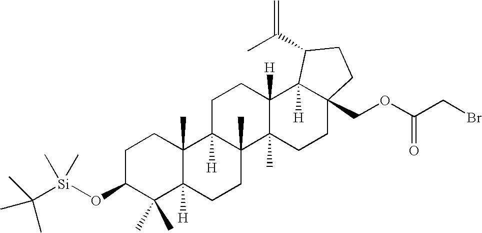 | ((1R,3aS,5aR,5bR,7aR,9S,11aR,11bR,13aR, 13bR)-9-(tert-butyldimethylsilyloxy)-5a,5b,8,8,11a- pentamethyl-1-(prop-1-en-2-yl)icosahydro-1H- cyclopenta11a]chrysen-3a-yl)methyl 2- bromoacetate |
| 13 | 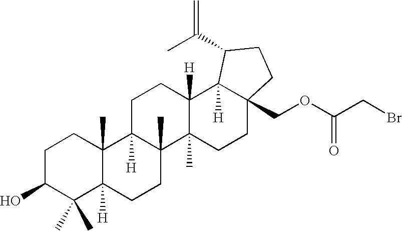 | ((1R,3aS,5aR,5bR,7aR,9S,11aR,11bR,13aR,13b R)-9-hydroxy-5a,5b,8,8,11a-pentamethyl-1- (prop-1-en-2-yl)icosahydro-1H- cyclopenta[a]chrysen-3a-yl)methyl 2- bromoacetate |
| 14 | 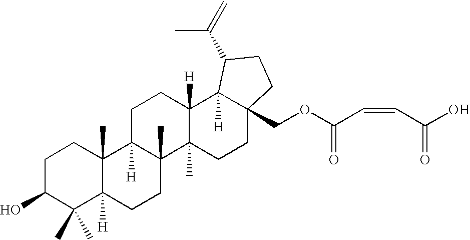 | (Z)-4- (((1R,3aS,5aR,5bR,7aR,9S,11aR,11bR,13aR,13b R)-9-hydroxy-5a,5b,8,8,11a-pentamethyl-1- (prop-1-en-2-yl)icosahydro-1H- cyclopenta[a]chrysen-3a-yl)methoxy)-4-oxobut- 2-enoic acid |
| 15 | 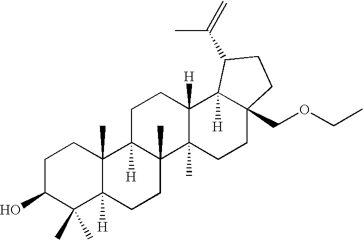 | (1R,3aS,5aR,5bR,7aR,9S,11aR,11bR,13aR,13bR)- 3a-(ethoxymethyl)-5a,5b,8,8,11a-pentamethyl- 1-(prop-1-en-2-yl)icosahydro-1H- cyclopenta[a]chrysen-9-ol |
| 16 | 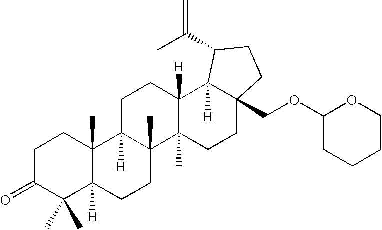 | (1R,3aS,5aR,5bR,7aR,11aR,11bR,13aR,13bR)- 5a,5b,8,8,11a-pentamethyl-1-(prop-1-en-2-yl)-3a- ((tetrahydro-2H-pyran-2- yloxy)methyl)octadecahydro-1H- cyclopenta[a]chrysen-9(5bH)-one |
| 17 | 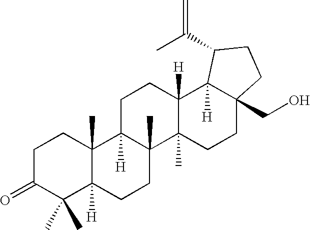 | (1R,3aS,5aR,5bR,7aR,11aR,11bR,13aR,13bR)- 3a-(hydroxymethyl)-5a,5b,8,8,11a-pentamethyl- 1-(prop-1-en-2-yl)octadecahydro-1H- cyclopenta[a]chrysen-9(5bH)-one |
| 18 | 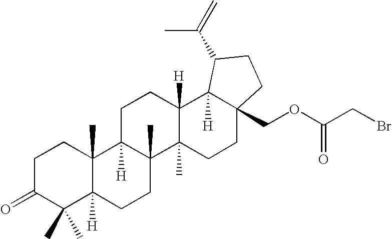 | ((1R,3aS,5aR,5bR,7aR,11aR,11bR,13aR,13bR)- 5a,5b,8,8,11a-pentamethyl-9-oxo-1-(prop-1-en-2- yl)icosahydro-1H-cyclopenta[a]chrysen-3a- yl)methyl 2-bromoacetate |
| 19 | 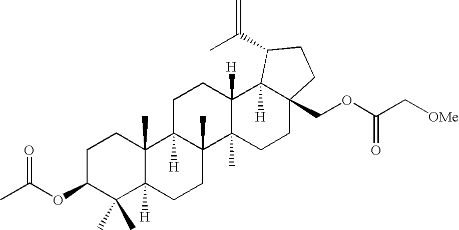 | ((1R,3aS,5aR,5bR,7aR,9S,11aR,11bR,13aR,13b R)-9-acetoxy-5a,5b,8,8,11a-pentamethyl-1-(prop- 1-en-2-yl)icosahydro-1H-cyclopenta[a]chrysen- 3a-yl)methyl 2-methoxyacetate |
| 20 | 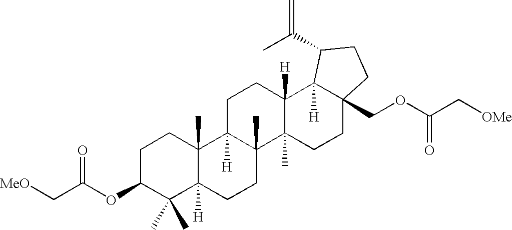 | ((1R,3aS,5aR,5bR,7aR,9S,11aR,11bR,13aR, 13bR)-9-(2-methoxyacetoxy)-5a,5b,8,8,11a- pentamethyl-1-(prop-1-en-2-yl)icosahydro-1H- cyclopenta[a]chrysen-3a-yl)methyl 2- methoxyacetate |
| 24 | 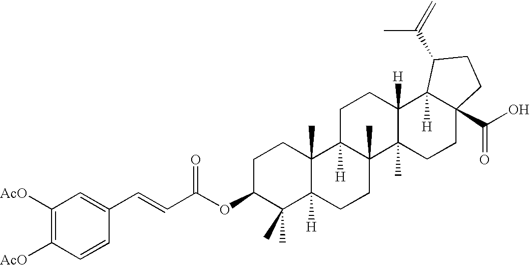 | (1R,3aS,5aR,5bR,7aR,9S,11aR,11bR,13aR, 13bR)-9-((E)-3-(3,4-diacetoxyphenyl)acryloyloxy)- 5a,5b,8,8,11a-pentamethyl-1-(prop-1-en-2- yl)icosahydro-1H-cyclopenta[a]chrysene-3a- carboxylic acid |
| 25 | 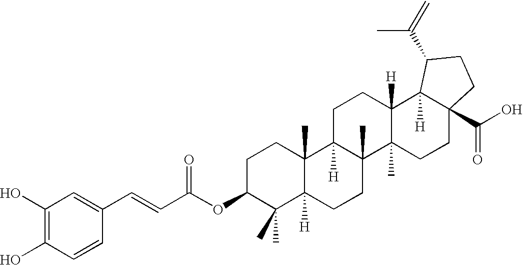 | (1R,3aS,5aR,5bR,7aR,9S,11aR,11bR,13aR, 13bR)-9-((E)-3-(3,4-dihydroxyphenyl)acryloyloxy)- 5a,5b,8,8,11a-pentamethyl-1-(prop-1-en-2- yl)icosahydro-1H-cyclopenta[a]chrysene-3a- carboxylic acid |
| 26 | 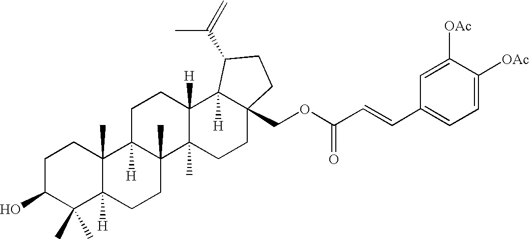 | (E)- ((1R,3aS,5aR,5bR,7aR,9S,11aR,11bR,13aR, 13bR)-9-hydroxy-5a,5b,8,8,11a-pentamethyl-1- (prop-1-en-2-yl)icosahydro-1H- cyclopenta[a]chrysen-3a-yl)methyl 3-(3,4- dihydroxyphenyl)acrylate |
| 27 | 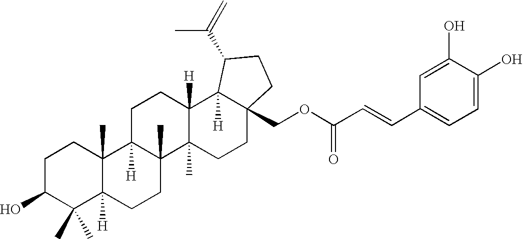 | (E)- ((1R,3aS,5aR,5bR,7aR,9S,11aR,11bR,13aR, 13bR)-9-hydroxy-5a,5b,8,8,11a-pentamethyl-1- (prop-1-en-2-yl)icosahydro-1H- cyclopenta[a]chrysen-3a-yl)methyl 3-(3,4- dihydroxyphenyl)acrylate |
| 28 | 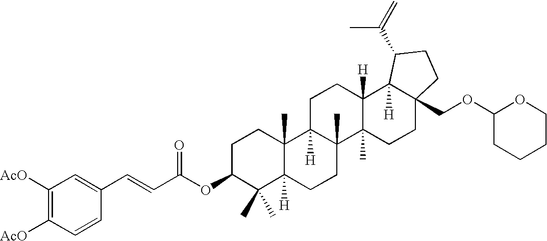 | 4-((E)-3-oxo-3- ((1R,3aS,5aR,5bR,7aR,9S,11aR,11bR,13aR,13b R)-5a,5b,8,8,11a-pentamethyl-1-(prop-1-en-2- yl)-3a-((tetrahydro-2H-pyran-2- yloxy)methy1)icosahydro-1H- cyciopenta[a]chrysen-9-yloxy)prop-1-enyl)-1,2- phenylene diacetate |
| 29 | 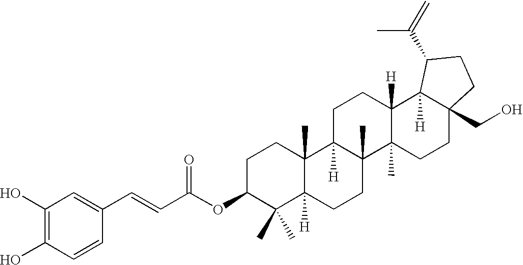 | (E)- ((1R,3aS,5aR,5bR,7aR,9S,11aR,11bR,13aR,13b R)-3a-(hydroxymethyl)-5a,5b,8,8,11a- pentamethyl-1-(prop-1-en-2-yl)icosahydro-1H- cyclopenta[a]chrysen-9-yl) 3-(3,4- dihydroxyphenyl)acrylate |
| 30 | 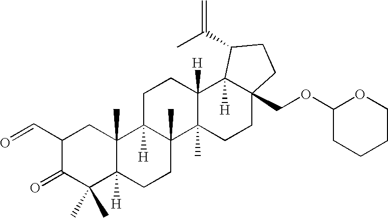 | (1R,3aS,5aR,5bR,7aR,11aR,11bR,13aR,13bR)- 5a,5b,8,8,11a-pentamethyl-9-oxo-1-(prop-1-en-2- yl)-3a-((tetrahydro-2H-pyran-2- yloxy)methyl)icosahydro-1H- cyclopenta[a]chrysene-10-carbaldehyde |
| 31 | 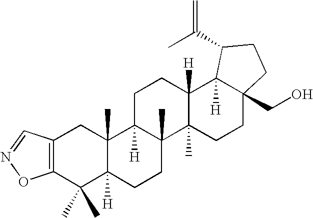 | [(1R,3aS,5aR,5bR,7aR,12aR,12bR,14aR,14bR)-1- isopropenyl-5a,5b,8,8,12a-pentamethyl- 1,2,3,4,5,5a,5b,6,7,7a,8,12,12a,12b,13,14,14a,14b- octadecahydro-3aH- cyclopenta[7,8]chryseno[3,2-d]isoxazol-3a- yl]methanol |
| 32 | 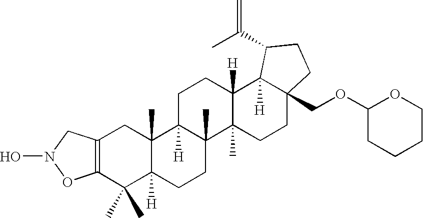 | (1R,3aS,5aR,5bR,7aR,12aR,12bR,14aR,14bR)-1- isopropenyl-5a,5b,8,8,12a-pentamethyl-3a- [(tetrahydro-2H-pyran-2-yloxy)methyl]- 1,2,3,3a,4,5,5a,5b,6,7,7a,8,11,12,12a,12b,13,14, 14a,14b-icosahydro-10H- cyclopenta[7,8]chryseno[3,2-d]isoxazol-10-ol |
| 33 | 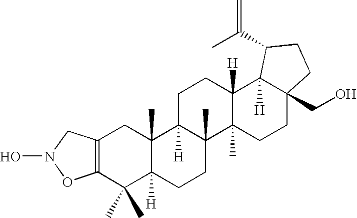 | (1R,3aS,5aR,5bR,7aR,12aR,12bR,14aR,14bR)-3a- (hydroxymethyl)-1-isopropenyl-5a,5b,8,8,12a- pentamethyl- 1,2,3,3a,4,5,5a,5b,6,7,7a,8,11,12,12a,12b,13,14, 14a,14b-icosahydro-10H- cyclopenta[7,8]chryseno[3,2-d]isoxazol-10-ol |
| 34 | 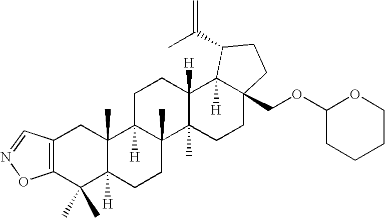 | (1R,3aS,5aR,5bR,7aR,12aR,12bR,14aR,14bR)-1- isopropenyl-5a,5b,8,8,12a-pentamethyl-3a- [(tetrahydro-2H-pyran-2-yloxy)methyl]- 2,3,3a,4,5,5a,5b,6,7,7a,8,12,12a,12b,13,14,14a, 14b-octadecahydro-1H- cyclopenta[7,8]chryseno[3,2-d]isoxazole |
| 35 | 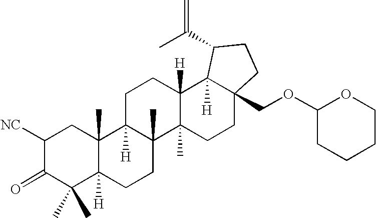 | (1R,3aS,5aR,5bR,7aR,11aR,11bR,13aR,13bR)- 5a,5b,8,8,11a-pentamethyl-9-oxo-1-(prop-1-en-2- yl)-3a-((tetrahydro-2H-pyran-2- yloxy)methyl)icosahydro-1H- cyclopenta[a]chrysene-10-carbonitrile |
| 36 | 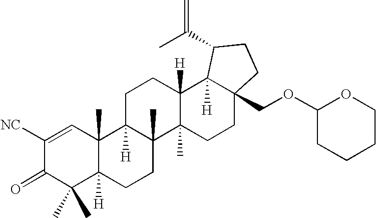 | (1R,3aS,5aR,5bR,7aR,11aR,11bR,13aR,13bR)- 5a,5b,8,8,11a-pentamethyl-9-oxo-1-(prop-1-en-2- yl)-3a-((tetrahydro-2H-pyran-2-yloxy)methyl)- 2,3,3a,4,5,5a,5b,6,7,7a,8,9,11a,11b,12,13,13a,13b- octadecahydro-1H-cyclopenta[a]chrysene-10- carbonitrile |
| 37 | 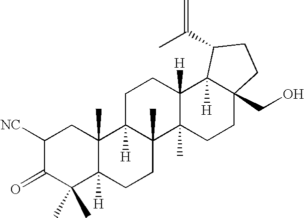 | (1R,3aS,5aR,5bR,7aR,11aR,11bR,13aR,13bR)- 3a-(hydroxymethyl)-5a,5b,8,8,11a-pentamethyl- 9-oxo-1-(prop-1-en-2-yl)icosahydro-1H- cyclopenta[a]chrysene-10-carbonitrile |
| 38 | 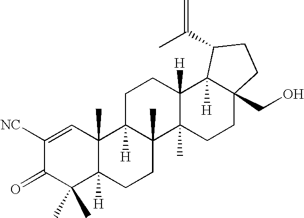 | (1R,3aS,5aR,5bR,7aR,11aR,11bR,13aR,13bR)- 3a-(hydroxymethyl)-5a,5b,8,8,11a-pentamethyl- 9-oxo-1-(prop-1-en-2-yl)-2,3,3a,4,5,5a,5b,6,7,7a, 8,9,11a,11b,12,13,13a,13b- octadecahydro-1H-cyclopenta[a]chrysene-10- carbonitrile |
| 39 | 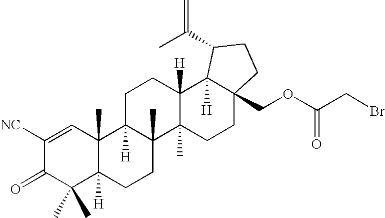 | ((1R,3aS,5aR,5bR,7aR,11aR,11bR,13aR,13bR)- 10-cyano-5a,5b,8,8,11a-pentamethyl-9-oxo-1- (prop-1-en-2-yl)- 2,3,3a,4,5,5a,5b,6,7,7a,8,9,11a,11b,12,13,13a,13b- octadecahydro-1H-cyclopenta[a]chrysen-3a- yl)methyl 2-bromoacetate |
| 40 | 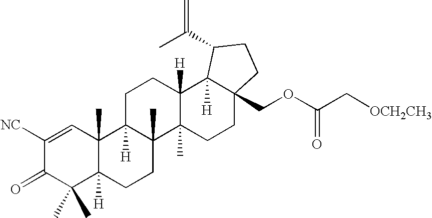 | ((1R,3aS,5aR,5bR,7aR,11aR,11bR,13aR,13bR)- 10-cyano-5a,5b,8,8,11a-pentamethyl-9-oxo-1- (prop-1-en-2-yl)- 2,3,3a,4,5,5a,5b,6,7,7a,8,9,11a,11b,12,13,13a,13b- octadecahydro-1H-cyclopenta[a]chrysen-3a- yl)methyl 2-ethoxyacetate |
| 41 | 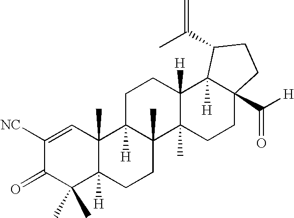 | (1R,3aS,5aR,5bR,7aR,11aR,11bR,13aR,13bR)- 3a-formyl-5a,5b,8,8,11a-pentamethyl-9-oxo-1- (prop-1-en-2-yl)- 2,3,3a,4,5,5a,5b,6,7,7a,8,9,11a,11b,12,13,13a,13b- octadecahydro-1H-cyclopenta[a]chrysene-10- carbonitrile |
| 42 | 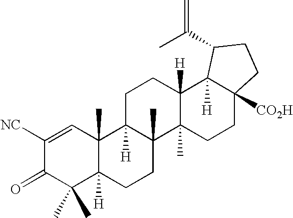 | (1R,3aS,5aR,5bR,7aR,11aR,11bR,13aR,13bR)- 10-cyano-5a,5b,8,8,11a-pentamethyl-9-oxo-1- (prop-1-en-2-yl)- 2,3,3a,4,5,5a,5b,6,7,7a,8,9,11a,11b,12,13,13a,13b- octadecahydro-1H-cyclopenta[a]chrysene-3a- carboxylic acid |
| 43 | 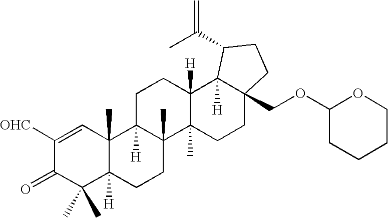 | (1R,3aS,5aR,5bR,7aR,11aR,11bR,13aR,13bR)- 5a,5b,8,8,11a-pentamethyl-9-oxo-1-(prop-1-en-2- yl)-3a-((tetrahydro-2H-pyran-2-yloxy)methyl)- 2,3,3a,4,5,5a,5b,6,7,7a,8,9,11a,11b,12,13,13a,13b- octadecahydro-1H-cyclopenta[a]chrysene-10- carbaldehyde |
| 44 | 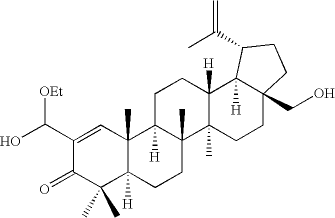 | (1R,3aS,5aR,5bR,7aR,11aR,11bR,13aR,13bR)- 10-(ethoxy(hydroxy)methyl)-3a- (hydroxymethyl)-5a,5b,8,8,11a-pentamethyl-1- (prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 45 | 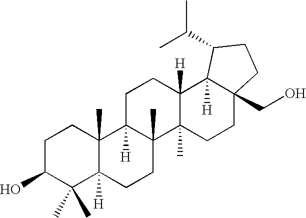 | (1S,3aS,5aR,5bR,7aR,9S,11aR,11bR,13aR,13bR)- 3a-(hydroxymethyl)-1-isopropyl-5a,5b,8,8,11a- pentamethylicosahydro-1H- cyclopenta[a]chrysen-9-ol |
| 46 | 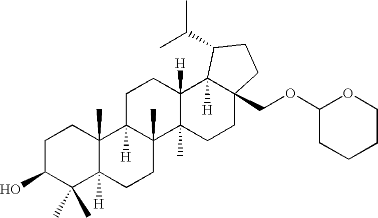 | (1S,3aS,5aR,5bR,7aR,9S,11aR,11bR,13aR,13bR)- 1-isopropyl-5a,5b,8,8,11a-pentamethyl-3a- ((tetrahydro-2H-pyran-2- yloxy)methyl)icosahydro-1H- cyclopenta[a]chrysen-9-ol |
| 47 | 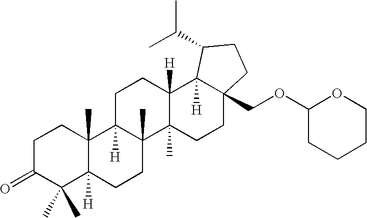 | (1S,3aS,5aR,5bR,7aR,11aR,11bR,13aR,13bR)-1- isopropyl-5a,5b,8,8,11a-pentamethyl-3a- ((tetrahydro-2H-pyran-2- yloxy)methyl)octadecahydro-1H- cyclopenta[a]chrysen-9(5bH)-one |
| 48 | 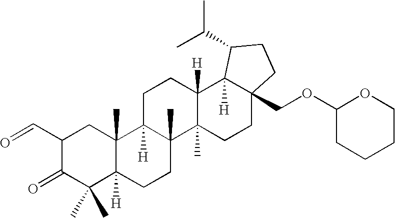 | (1S,3aS,5aR,5bR,7aR,11aR,11bR,13aR,13bR)-1- isopropyl-Sa,5b,8,8,11a-pentamethyl-9-oxo-3a- ((tetrahydro-2H-pyran-2- yloxy)methyl)icosahydro-1H- cyclopenta[a]chrysene-10-carbaldehyde |
| 49 | 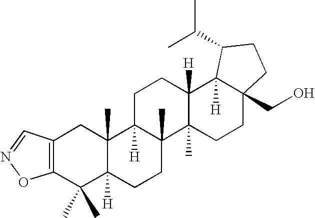 | [(1S,3aS,5aR,5bR,7aR,12aR,12bR,14aR,14bR)-1- isopropyl-5a,5b,8,8,12a-pentamethyl- 1,2,3,4,5,5a,5b,6,7,7a,8,12,12a,12b,13,14,14a, 14b-octadecahydro-3aH- cyclopenta[7,8]chryseno[3,2-d]isoxazol-3a- yl]methanol |
| 50 | 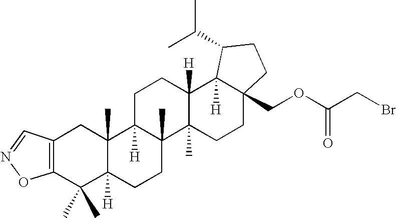 | [(1S,3aS,5aR,5bR,7aR,12aR,12bR,14aR,14bR)-1- isopropyl-5a,5b,8,8,12a-pentamethyl- 1,2,3,4,5,5a,5b,6,7,7a,8,12,12a,12b,13,14,14a, 14b-octadecahydro-3aH- cyclopenta[7,8]chryseno[3,2-d]isoxazol-3a- yl]methyl bromoacetate |
| 51 | 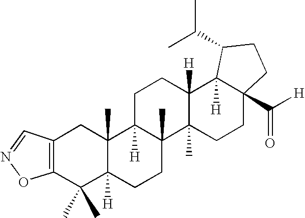 | (1S,3aS,5aR,5bR,7aR,12aR,12bR,14aR,14bR)-1- isopropyl-5a,5b,8,8,12a-pentamethyl- 1,2,3,4,5,5a,5b,6,7,7a,8,12,12a,12b,13,14,14a, 14b-octadecahydro-3aH- cyclopenta[7,8]chryseno[3,2-d]isoxazole-3a- carbaldehyde |
| 52 | 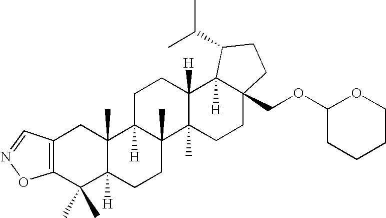 | (1S,3aS,5aR,5bR,7aR,12aR,12bR,14aR,14bR)-1- isopropyl-5a,5b,8,8,12a-pentamethyl-3a- [(tetrahydro-2H-pyran-2-yloxy)methyl]- 2,3,3a,4,5,5a,5b,6,7,7a,8,12,12a,12b,13,14,14a, 14b-octadecahydro-1H- cyclopenta[7,8]chryseno[3,2-d]isoxazole |
| 53 | 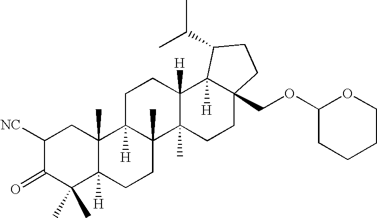 | (1S,3aS,5aR,5bR,7aR,11aR,11bR,13aR,13bR)-1- isopropyl-5a,5b,8,8,11a-pentamethyl-9-oxo-3a- ((tetrahydro-2H-pyran-2- yloxy)methyl)icosahydro-1H- cyclopenta[a]chrysene-10-carbonitrile |
| 54 | 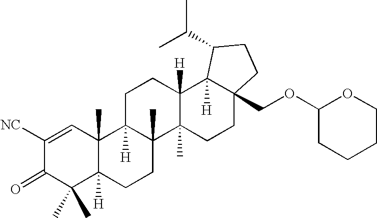 | (1S,3aS,5aR,5bR,7aR,11aR,11bR,13aR,13bR)-1- isopropyl-5a,5b,8,8,11a-pentamethyl-9-oxo-3a- ((tetrahydro-2H-pyran-2-yloxy)methyl)- 2,3,3a,4,5,5a,5b,6,7,7a,8,9,11a,11b,12,13,13a,13b- octadecahydro-1H-cyclopenta[a]chrysene-10- carbonitrile |
| 55 | 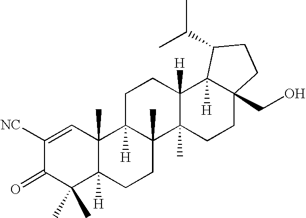 | (1S,3aS,5aR,5bR,7aR,11aR,11bR,13aR,13bR)- 3a-(hydroxymethyl)-1-isopropyl-5a,5b,8,8,11a- pentamethyl-9-oxo- 2,3,3a,4,5,5a,5b,6,7,7a,8,9,11a,11b,12,13,13a,13b- octadecahydro-1H-cyclopenta[a]chrysene-10- carbonitrile |
| 56 | 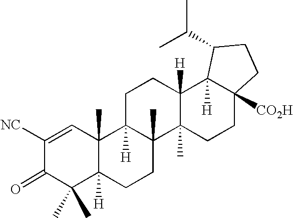 | (1S,3aS,SaR,5bR,7aR,11aR,11bR,13aR,13bR)- 10-cyano-1-isopropyl-5a,5b,8,8,11a-pentamethyl- 9-oxo- 2,3,3a,4,5,5a,5b,6,7,7a,8,9,11a,11b,12,13,13a,13b- octadecahydro-1H-cyclopenta[a]chrysene-3a- carboxylic acid |
| 57 | 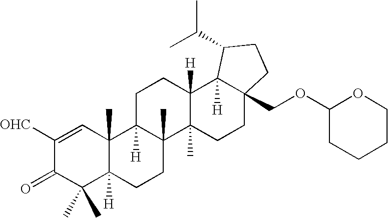 | (1S,3aS,5aR,5bR,7aR,11aR,11bR,13aR,13bR)-1- isopropyl-5a,5b,8,8,11a-pentamethyl-9-oxo-3a- ((tetrahydro-2H-pyran-2-yloxy)methyl)- 2,3,3a,4,5,5a,5b,6,7,7a,8,9,11a,11b,12,13,13a,13b- octadecahydro-1H-cyclopenta[a]chrysene-10- carbaldehyde |
| 58a | 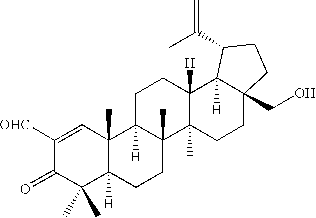 | (1R,3aS,SaR,5bR,7aR,11aR,11bR,13aR,13bR)- 3a-(hydroxymethyl)-5a,5b,8,8,11a-pentamethyl- 9-oxo-1-(prop-1-en-2-yl)- 2,3,3a,4,5,5a,5b,6,7,7a,8,9,11a,11b,12,13,13a,13b- octadecahydro-1H-cyclopenta[a]chrysene-10- carbaldehyde |
| 58b | 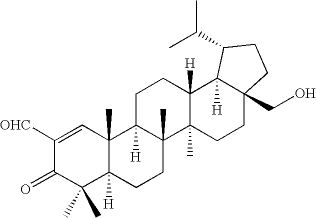 | (1S,3aS,5aR,5bR,7aR,11aR,11bR,13aR,13bR)- 3a-(hydroxymethyl)-1-isopropyl-5a,5b,8,8,11a- pentamethyl-9-oxo- 2,3,3a,4,5,5a,5b,6,7,7a,8,9,11a,11b,12,13,13a,13b- octadecahydro-1H-cyclopenta[a]chrysene-10- carbaldehyde |
| 59a | 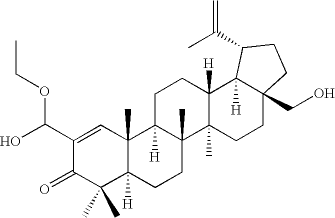 | (1R,3aS,5aR,5bR,7aR,11aR,11bR,13aR,13bR)- 10-(ethoxy(hydroxy)methyl)-3a- (hydroxymethyl)-5a,5b,8,8,11a-pentamethyl-1- (prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 59b | 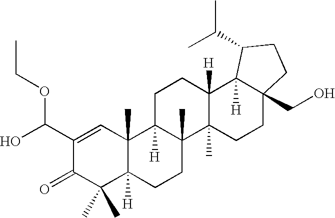 | (1S,3aS,5aR,5bR,7aR,11aR,11bR,13aR,13bR)- 10-(ethoxy(hydroxy)meihyl)-3a- (hydroxymethyl)-1-isopropyl-5a,5b,8,8,11a- pentamethyl- 2,3,3a,4,5,5a,6,7,7a,8,11a,11b,12,13,13a,13b- hexadecahydro-1H-cyclopenta[a]chrysen- 9(5bH)-one |
| 60a | 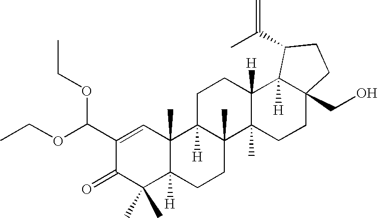 | (1R,3aS,5aR,5bR,7aR,11aR,11bR,13aR,13bR)- 10-(diethoxymethyl)-3a-(hydroxymethyl)- 5a,5b,8,8,11a-pentamethyl-1-(prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 60b | 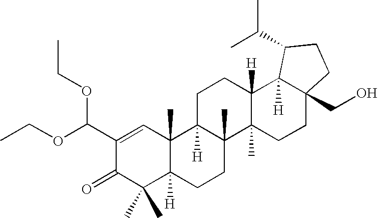 | (1S,3aS,5aR,5bR,7aR,11aR,11bR,13aR,13bR)- 10-(diethoxymethyl)-3a-(hydroxymethyl)-1- isopropyl-5a,5b,8,8,11a-pentamethyl- 2,3,3a,4,5,5a,6,7,7a,8,11a,11b,12,13,13a,13b- hexadecahydro-1H-cyclopenta[a]chrysen- 9(5bH)-one |
| 61 | 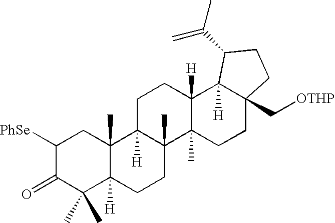 | (1R,3aS,5aR,5bR,7aR,11aR,11bR,13aR,13bR)- 5a,5b,8,8,11a-pentamethyl-10-(phenylselanyl)-1- (prop-1-en-2-yl)-3a-((tetrahydro-2H-pyran-2- yloxy)methyl)octadecahydro-1H- cyclopenta[a]chrysen-9(5bH)-one |
| 62 | 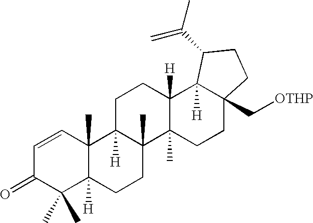 | (1R,3aS,5aR,5bR,7aR,11aR,11bR,13aR,13bR)- 5a,5b,8,8,11a-pentamethyl-1-(prop-1-en-2-yl)-3a- ((tetrahydro-2H-pyran-2-yloxy)methyl)- 2,3,3a,4,5,5a,6,7,7a,8,11a,11b,12,13,13a,13b- hexadecahydro-1H-cyclopenta[a]chrysen- 9(5bH)-one |
| 63 | 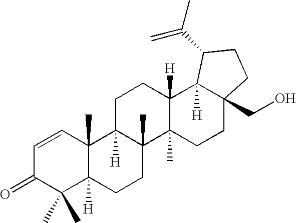 | (1R,3aS,5aR,5bR,7aR,11aR,11bR,13aR,13bR)- 3a-(hydroxymethyl)-5a,5b,8,8,11a-pentamethyl- 1-(prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 64a | 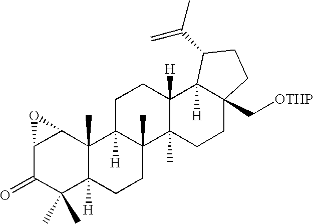 | (1aR,3aR,5aR,5bR,7aS,10R,10aR,10bR,12aS, 12bR,12cR)-10-isopropenyl-3,3,5a,5b,12b- pentamethyl-7a-[(tetrahydro-2H-pyran-2- yloxy)methyl]icosahydro-2H- cyclopenta[7,8]chryseno[3,4-b]oxiren-2-one |
| 64b | 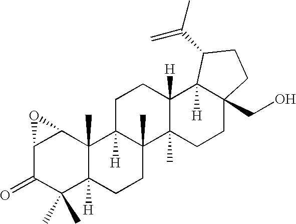 | (1aR,3aR,5aR,5bR,7aS,10R,10aR,10bR,12aS, 12bR)-7a-(hydroxymethyl)-10-isopropenyl- 3,3,5a,5b,12b-pentamethylicosahydro-2H- cyclopenta[7,8]chryseno[3,4-b]oxiren-2-one |
| 64c | 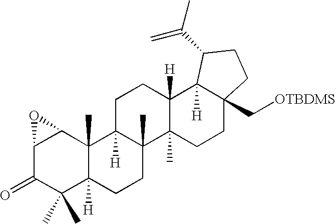 | (1aR,3aR,5aR,5bR,7aS,10R,10aR,10bR,12aS, 12bR,12cR)-7a-({[tert- butyl(dimethyl)silyl]oxy}methyl)-10- isopropenyl-3,3,5a,5b,12b- pentamethylicosahydro-2H- cyclopenta[7,8]chryseno [3,4-b]oxiren-2-one |
| 65a | 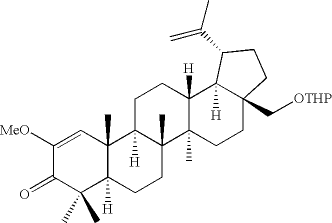 | (1R,3aS,5aR,5bR,7aR,11aR,11bR,13aR,13bR)- 10-methoxy-5a,5b,8,8,11a-pentamethyl-1-(prop- 1-en-2-yl)-3a-((tetrahydro-2H-pyran-2- yloxy)methyl)- 2,3,3a,4,5,5a,6,7,7a,8,11a,11b,12,13,13a,13b- hexadecahydro-1H-cyclopenta[a]chrysen- 9(5bH)-one |
| 65b | 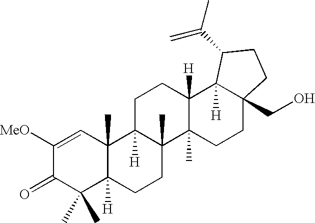 | (1R,3aS,5aR,5bR,7aR,11aR,11bR,13aR,13bR)- 3a-(hydroxymethyl)-10-methoxy-5a,5b,8,8,11a- pentamethyl-1-(prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 65c | 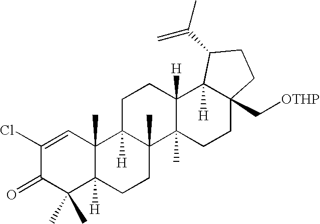 | (1R,3aS,5aR,5bR,7aR,11aR,11bR,13aR,13bR)- 10-chloro-5a,5b,8,8,11a-pentamethyl-1-(prop-1- en-2-yl)-3a-((tetrahydro-2H-pyran-2- yloxy)methyl)- 2,3,3a,4,5,5a,6,7,7a,8,11a,11b,12,13,13a,13b- hexadecahydro-1H-cyclopenta[a]chrysen- 9(5bH)-one |
| 65d | 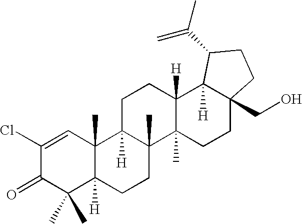 | (1R,3aS,5aR,5bR,7aR,11aR,11bR,13aR,13bR)- 10-chloro-3a-(hydroxymethyl)-5a,5b,8,8,11a- pentamethyl-1-(prop-1-en-2-yl)- 2,3,3a,4,5,5a,6,7,7a,8,11a,11b,12,13,13a,13b- hexadecahydro-1H-cyclopenta[a]chrysen- 9(5bH)-one |
| 66 | 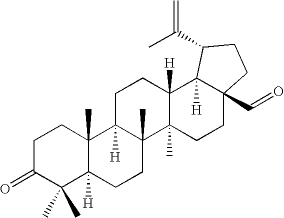 | (1R,3aS,5aR,5bR,7aR,11aR,11bR,13aR,13bR)- 5a,5b,8,8,11a-pentamethyl-9-oxo-1-(prop-1-en-2- yl)icosahydro-1H-cyclopenta[a]chrysene-3a- carbaldehyde |
| 67 | 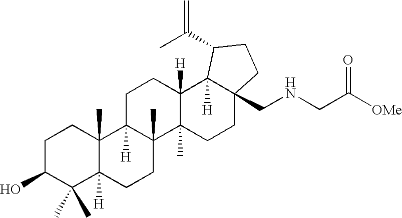 | methyl 2- (((1R,3aS,5aR,5bR,7aR,9S,11aR,11bR,13aR,13b R)-9-hydroxy-5a,5b,8,8,11a-pentamethyl-1- (prop-1-en-2-yl)icosahydro-1H- cyclopenta[a]chrysen-3a-yl)methylamino)acetate |
| 68 | 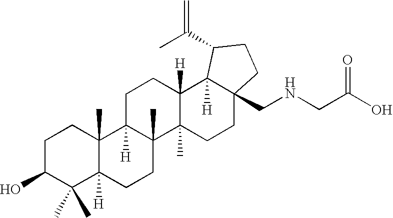 | 2- (((1R,3aS,5aR,5bR,7aR,9S,11aR,11bR,13aR,13bR)- 9-hydroxy-5a,5b,8,8,11a-pentamethyl-1- (prop-1-en-2-yl)icosahydro-1H- cyclopenta[a]chrysen-3a-yl)methylamino)acetic acid |
| 69 | 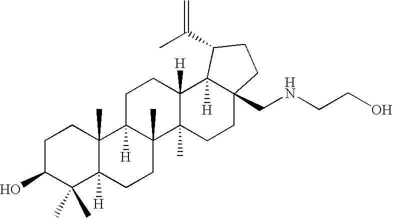 | (1R,3aS,5aR,5bR,7aR,9S,11aR,11bR,13aR,13bR)- 3a-((2-hydroxyethylamino)methyl)- 5a,5b,8,8,11a-pentamethyl-1-(prop-1-en-2- yl)icosahydro-1H-cyclopenta[a]chrysen-9-ol |
| 70 | 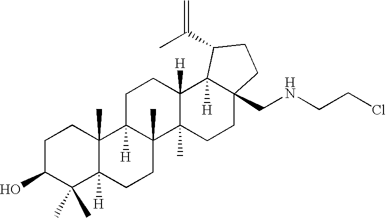 | (1R,3aS,5aR,5bR,7aR,9S,11aR,11bR,13aR,13bR)- 3a-((2-chloroethylamino)methyl)-5a,5b,8,8,11a- pentamethyl-1-(prop-1-en-2-yl)icosahydro-1H- cyclopenta[a]chrysen-9-ol |
| 71 | 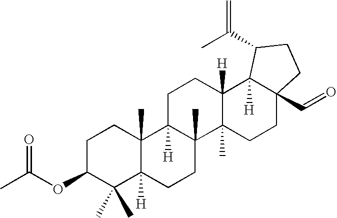 | (1R,3aS,5aR,5bR,7aR,9S,11aR,11bR,13aR,13bR)- 3a-formyl-5a,5b,8,8,11a-pentamethyl-1-(prop- 1-en-2-yl)icosahydro-1H-cyclopenta[a]chrysen- 9-ylacetate |
| 72 | 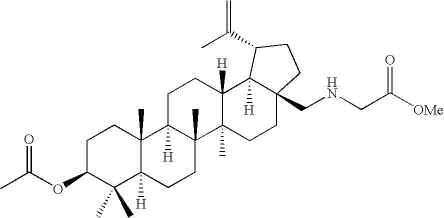 | methyl 2- (((1R,3aS,5aR,5bR,7aR,9S,1iaR,11bR,13aR,13bR)- 9-acetoxy-5a,5b,8,8,11a-pentamethyl-1-(prop- 1-en-2-yl)icosahydro-1H-cyclopenta[a]chrysen- 3a-yl)methylamino)acetate |
| 73 | 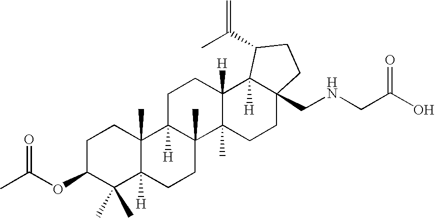 | (1R,3aS,5aR,5bR,7aR,9S,11aR,11bR,13aR,13bR)- 3a-((2-hydroxyethylamino)methyl)- 5a,5b,8,8,11a-pentamethyl-1-(prop-1-en-2- yl)icosahydro-1H-cyclopenta[a]chrysen-9-yl acetate |
| 74 | 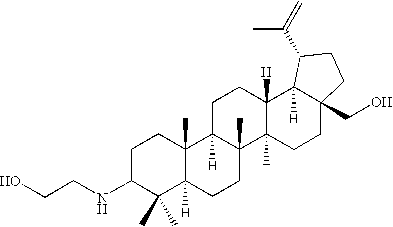 | 2- ((1R,3aS,5aR,5bR,7aR,11aR,11bR,13aR,13bR)- 3a-(hydroxymethyl)-5a,5b,8,8,11a-pentamethyl- 1-(prop-1-en-2-yl)icosahydro-1H- cyclopenta[a]chrysen-9-ylamino)ethanol |
| 75 | 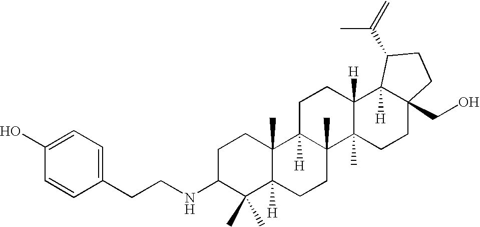 | 4-(2- ((1R,3aS,5aR,5bR,7aR,11aR,11bR,13aR,13bR)- 3a-(hydroxymethyl)-5a,5b,8,8,11a-pentamethyl- 1-(prop-1-en-2-yl)icosahydro-1H- cyclopenta[a]chrysen-9-ylamino)ethyl)phenol |
| 76 | 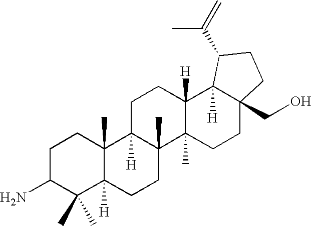 | ((1R,3aS,5aR,5bR,7aR,1iaR,11bR,13aR,13bR)- 9-amino-5a,5b,8,8,11a-pentamethyl-1-(prop-1- en-2-yl)icosahydro-1H-cyclopenta[a]chrysen-3a- yl)methanol |
| 77 | 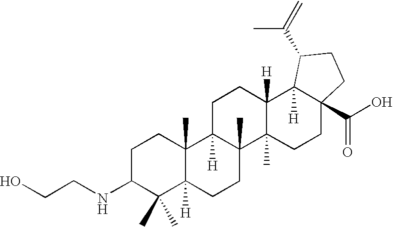 | (1R,3aS,5aR,5bR,7aR,11aR,11bR,13aR,13bR)-9- (2-hydroxyethylamino)-5a,5b,8,8,11a- pentamethyl-1-(prop-1-en-2-yl)icosahydro-1H- cyclopenta[a]chrysene-3a-carboxylic acid |
| 78 | 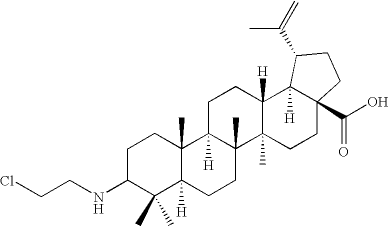 | (1R,3aS,5aR,5bR,7aR,11aR,11bR,13aR,13bR)-9- (2-chloroethylamino)-5a,5b,8,8,11a-pentamethyl- 1-(prop-1-en-2-yl)icosahydro-1H- cyclopenta[a]chrysene-3a-carboxylic acid |
| 79 | 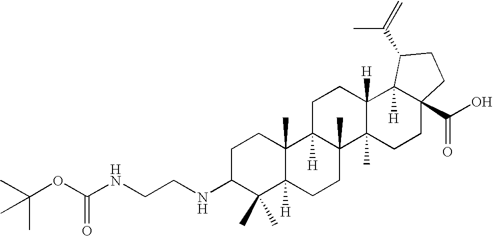 | (1R,3aS,5aR,5bR,7aR,11aR,11bR,13aR,13bR)-9- (2-(tert-butoxycarbonylamino)ethylamino)- 5a,5b,8,8,11a-pentamethyl-1-(prop-1-en-2- yl)icosahydro-1H-cyclopenta[a]chrysene-3a- carboxylic acid |
| 80 | 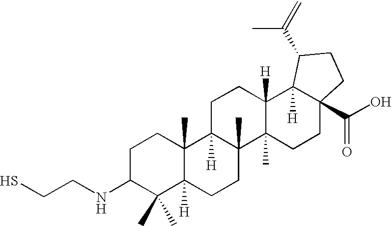 | (1R,3aS,5aR,5bR,7aR,11aR,11bR,13aR,13bR)-9- (2-mercaptoethylamino)-5a,5b,8,8,11a- pentamethyl-1-(prop-1-en-2-yl)icosahydro-1H- cyclopenta[a]chrysene-3a-carboxylic acid |
| 81 | 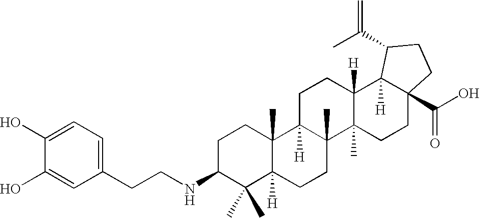 | (1R,3aS,5aR,5bR,7aR,9S,11aR,11bR,13aR,13bR)- 9-(3,4-dihydroxyphenethylamino)- 5a,5b,8,8,11a-pentamethyl-1-(prop-1-en-2- yl)icosahydro-1H-cyclopenta[a]chrysene-3a- carboxylic acid |
| 82 | 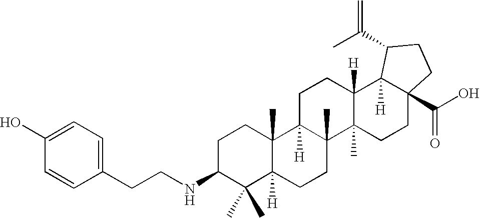 | (1R,3aS,5aR,5bR,7aR,9S,11aR,11bR,13aR,13bR)- 9-(4-hydroxyphenethylamino)-5a,5b,8,8,11a- pentamethyl-1-(prop-1-en-2-yl)icosahydro-1H- cyclopenta[a]chrysene-3a-carboxylic acid |
| 83 | 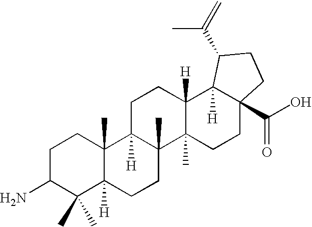 | (1R,3aS,5aR,5bR,7aR,11aR,11bR,13aR,13bR)-9- amino-5a,5b,8,8,11a-pentamethyl-1-(prop-1-en- 2-yl)icosahydro-1H-cyclopenta[a]chrysene-3a- carboxylic acid |
| 84 | 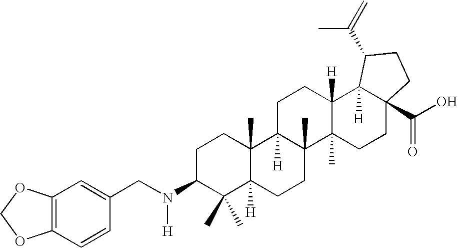 | (1R,3aS,5aR,5bR,7aR,9S,11aR,11bR,13aR,13bR)- 9-(benzo [d][1,3]dioxol-5 -ylmethylamino)- 5a,5b,8,8,11a-pentamethyl-1-(prop-1-en-2- yl)icosahydro-1H-cyclopenta[a]chrysene-3a- carboxylic acid |
| 85 | 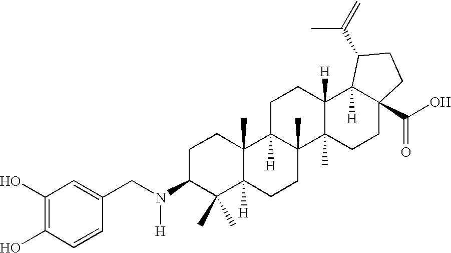 | (1R,3aS,5aR,5bR,7aR,9S,11aR,11bR,13aR,13bR)- 9-(3,4-dihydroxybenzylamino)-5a,5b,8,8,11a- pentamethyl-1-(prop-1-en-2-yl)icosahydro-1H- cyclopenta[a]chrysene-3a-carboxylic acid |
| 86 | 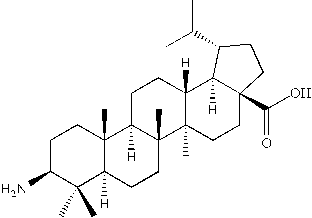 | (1S,3aS,5aR,5bR,7aR,9S,11aR,11bR,13aR,13bR)- 9-amino-1-isopropyl-5a,5b,8,8,11a- pentamethylicosahydro-1H- cyclopenta[a]chrysene-3a-carboxylic acid |
| 90 | 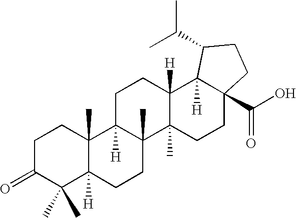 | (1S,3aS,5aR,5bR,7aR,11aR,11bR,13aR,13bR)-1- isopropyl-5a,5b,8,8,11a-pentamethyl-9- oxoicosahydro-1H-cyclopenta[a]chrysene-3a- carboxylic acid |

### Synthetic Procedures

In order to derivatize betulin at 28-position, 3-O-protected betulin (7 and 11) is required and can be synthesized from betulin in three steps. Selective THP protection of the primary alcohol at the 28-position is followed by acylation or silylation at C-3 and the deprotection of the THP group to free the 28-OH.

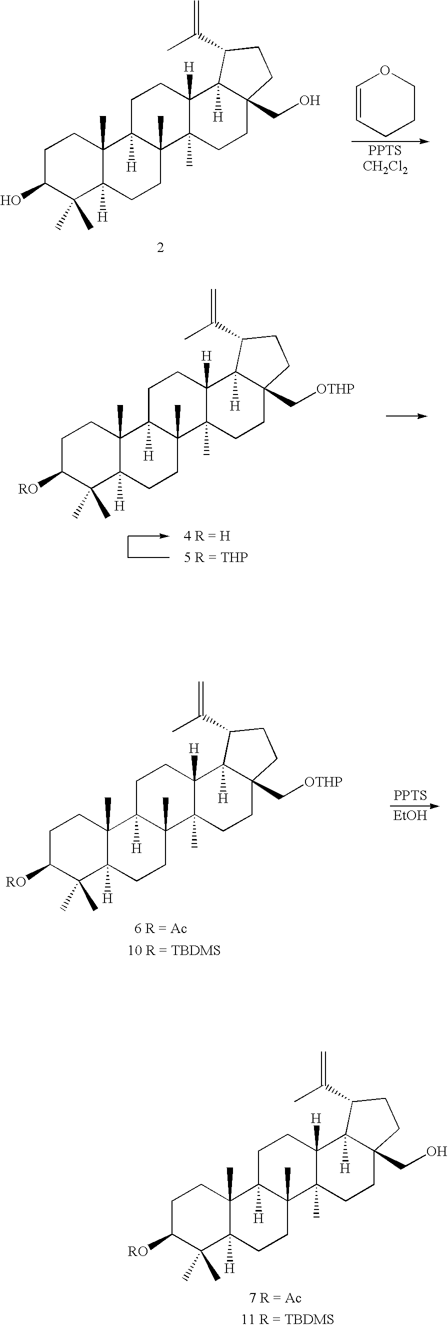

In the THP protection step, 3,28-bis-O-THP-betulin (5) was also formed. In the presence of catalytic amount of acids, the 3-O-THP group can be selectively removed to furnish the 28-O-THP betulin 4 in moderate yields. Thus, this procedure can be utilized to recycle the byproduct 3,28-bis-O-THP-betulin (5).

The O-alkylation of betulin with ethyl iodide in the presence of NaH was achieved selectively at the 28-position to yield 15. When ethyl bromoacetate was used as the alkylating agent, however, the 28-O-acylation, instead of the O-alkylation, took place to generate the corresponding bromoacetate (13). Addition of phase transfer catalyst TBAI did not alter the reaction outcome.

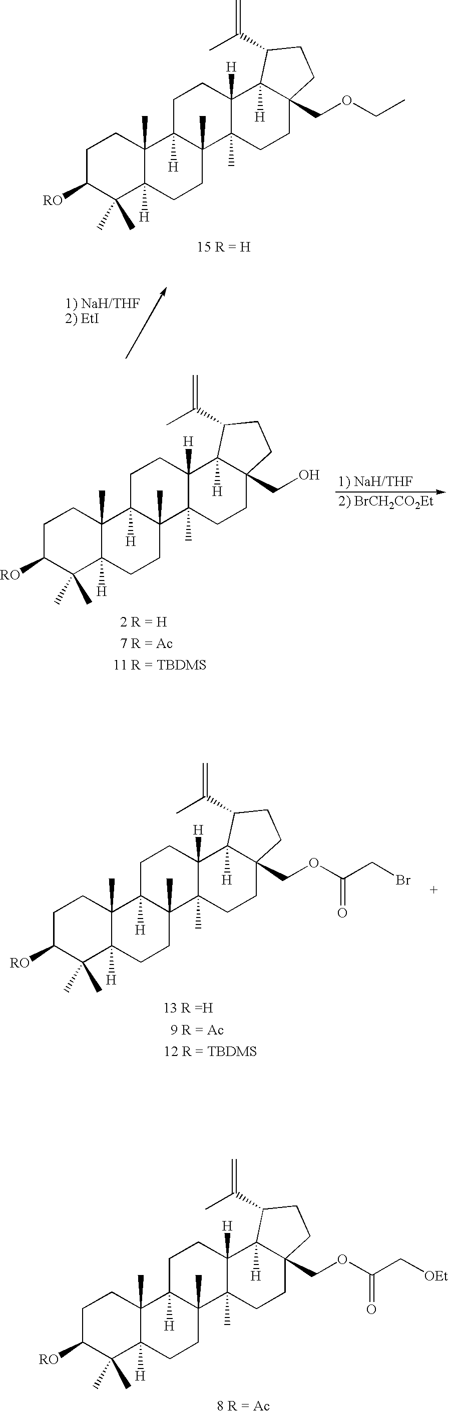

Similarly, reaction of 3-β-acetoxy-betulin (7) and 3-β-O-TBDMS-betulin (11) with ethyl bromoacetate provided two unexpected 28-O-acyl products 9 and 12 respectively. Interestingly, the ethyl ester 8 was also recovered in conjunction with bromoacetyl 9. The alkaline hydrolysis of 8 and 9 at room temperature furnished back 3-β-acetoxybetulin (7). 28-O-Acylation of betulone 17, prepared from 4 by Jones oxidation and deprotection of THP group, was also achieved with ethyl bromoacetate to afford 3-oxo-28-bromoacetyl ester 18.

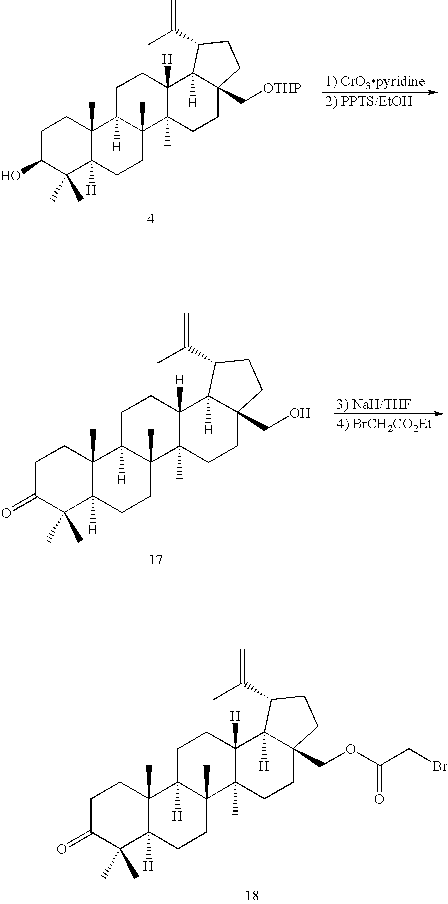

Direct acylation of 3-β-acetylbetulin 7 with bromoacetyl chloride produced a 2-component mixture. Attempts to separate and purify the products proved unsuccessful. However, ¹H NMR spectrum indicated that the major component was compound 9.

The preference of O-acylation at 28-position was further confirmed by reaction of 3-β-acetylbetulin 7 with methoxy acetyl chloride. The product thus formed was the 28-methoxyacetate 19, which demonstrated similar chemical shift frequency and multiplicity patterns in its ¹H NMR spectrum for CH₂OCO and COCH₂OMe protons as those observed in 8, 9, 12 and 13.

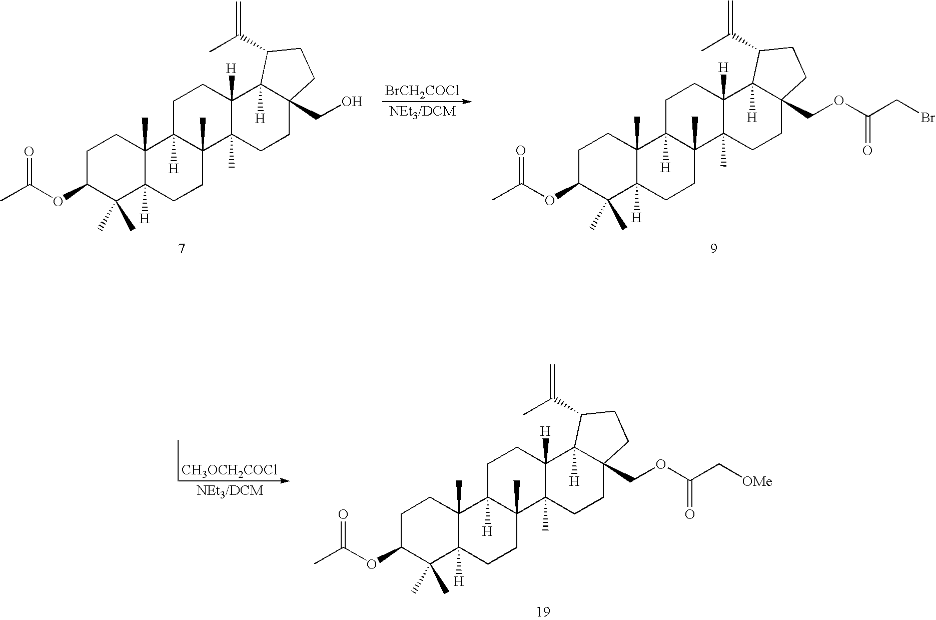

Reaction of unprotected betulin with methoxy acetyl chloride under the same conditions produced the bis-methoxy acetyl analogue 20. However, when 3,4-diacetoxycaffeic acid chloride and maleic anhydride were used as the acylating reagents, only the 28-O-acyl products 14 and 27 were isolated.

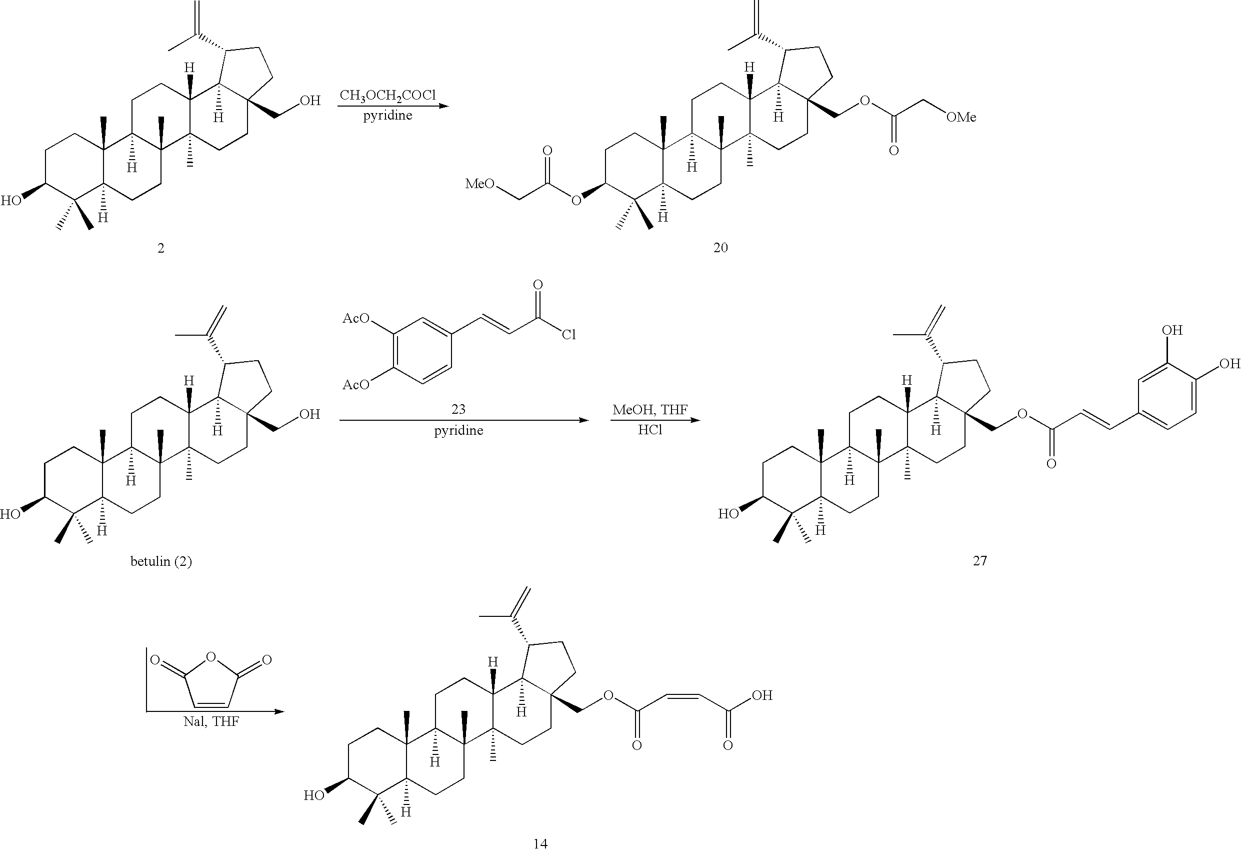

3-O-Acylation is achieved when 28-OH is protected (e.g., 4) or masked as other functional groups (e.g., 1). Thus, reaction of betulinic acid (1) or 28-O-THP-betulin (4) with 3,4-diacetoxycaffeic acid chloride, followed by acid catalyzed removal of acetyl groups, furnished the corresponding caffeic acid ester derivatives 25 and 29, respectively.

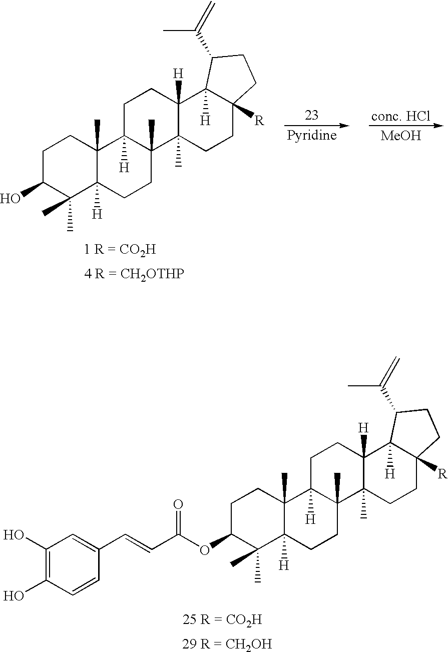

Further modifications on the A-ring of betulinic acid and betulin are described below. 28-O-THP-Betulin (4) and 28-O-THP-dihydrobetulin (46) were converted to the corresponding isoxazoles 34 or 52 in four steps by oxidation (CrO₃.pyridine) at 3-OH followed by α-formylation of the resulting ketones (HCO₂Et, NaOMe) and cyclization to isoxazole intermediates 31 and 49 (NH₂OH.HCl, EtOH). The THP protecting groups were not stable under slightly acidic conditions and were reassembled to afford the corresponding C-28 protected isoxazoles respectively. Cleavage of N—O bond in 34 or 52 was affected by deprotonation of the isoxazole rings (NaOMe, toluene). Oxidation of the resulting α-cyano ketones 35 and 53 at C I-C2 bond promoted by DDQ followed by removal of THP groups provided the corresponding α-β-unsaturated-α-cyano keto alcohols 38 and 55 in good yields.

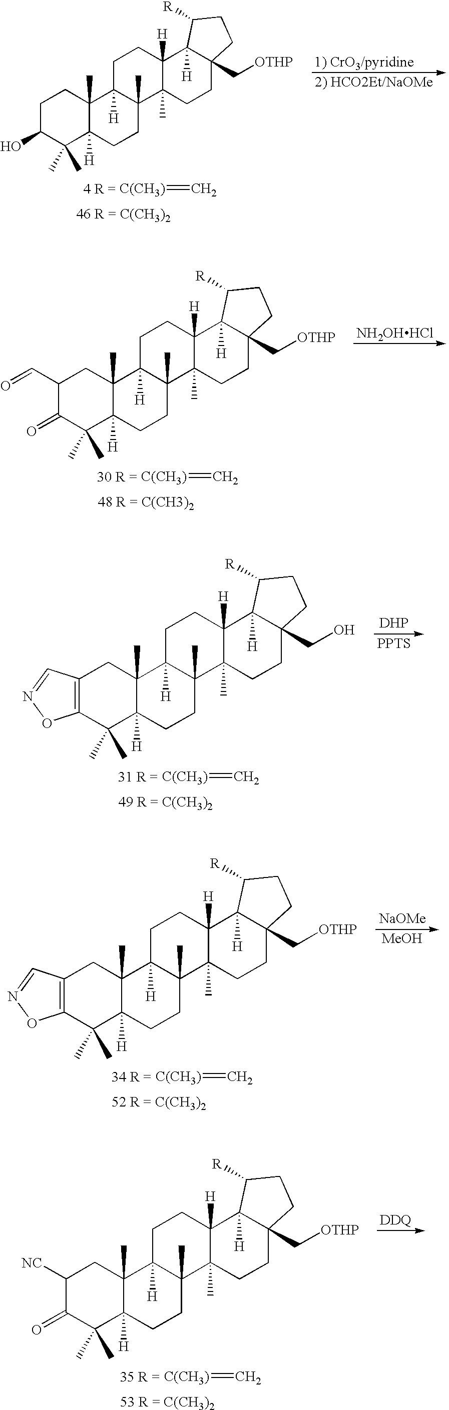

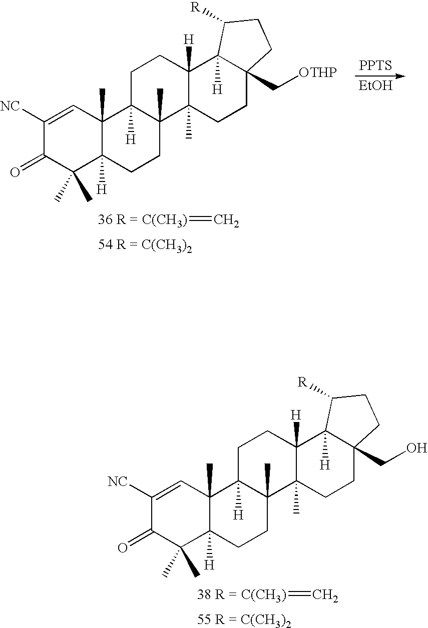

28-O-Acylation of 38 and 49 with ethyl bromoacetate in the presence of NaH afforded the A-ring modified derivatives 39, 40 and 50.

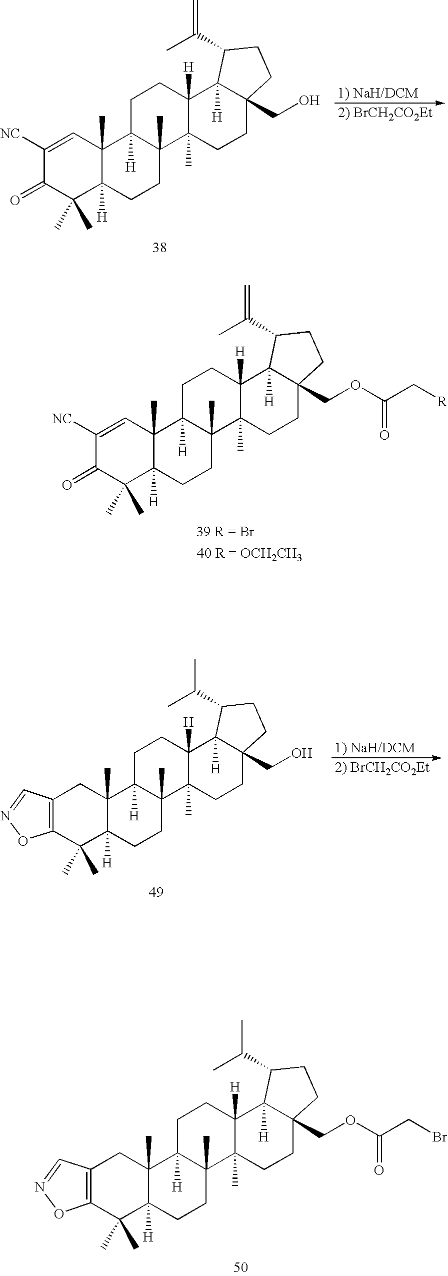

Cyclization reaction of keto aldehyde 30 under basic conditions (NH₂OH.HCl/KOH) was investigated and an N—OH isoxazole was isolated which was deprotected at C-28 to afford 33.

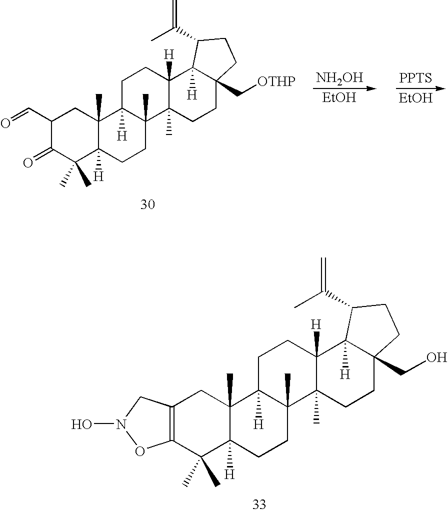

Oxidation of α-cyano ketone derivative 38 and isoxazole 49 by CrO₃.pyridine yielded the corresponding aldehyde derivatives 41 and 51 respectively. However, oxidation of 38 and 55 by Jones' reagent (CrO₃.H₂SO₄) provided the corresponding betulinic acid derivatives 42 and 56 respectively.

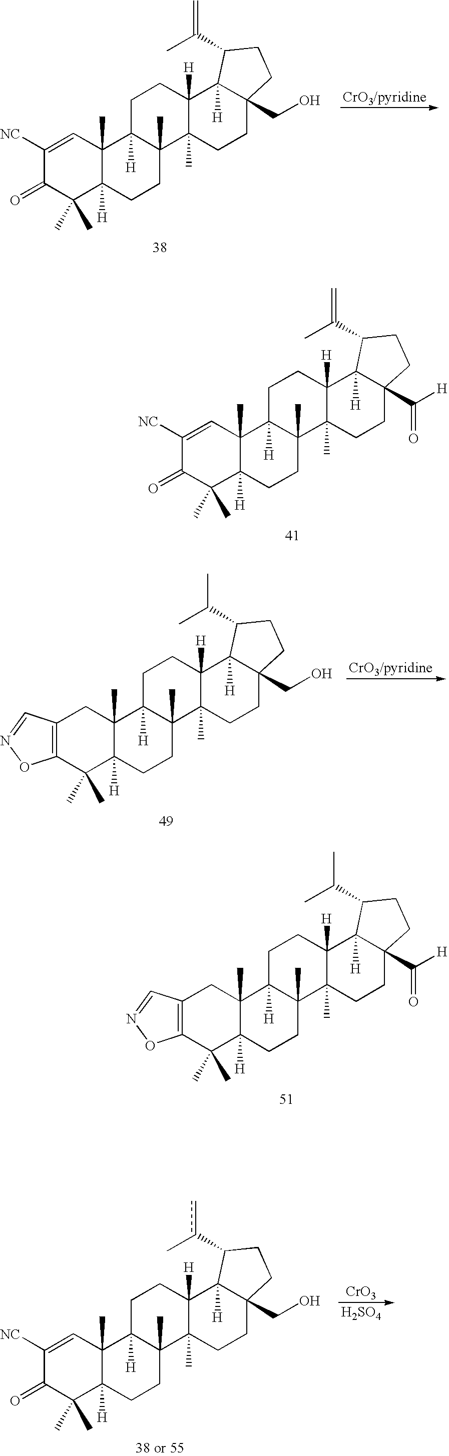

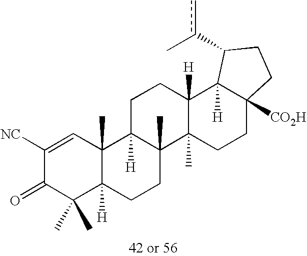

Dehydrogenation of 2-aldyhed derivatives 30 and 48 using DDQ resulted in the corresponding enone analogues 43 and 57. Removal of THP in EtOH afforded compounds 58, along with the semi-acetal and acetal compounds 59 and 60.

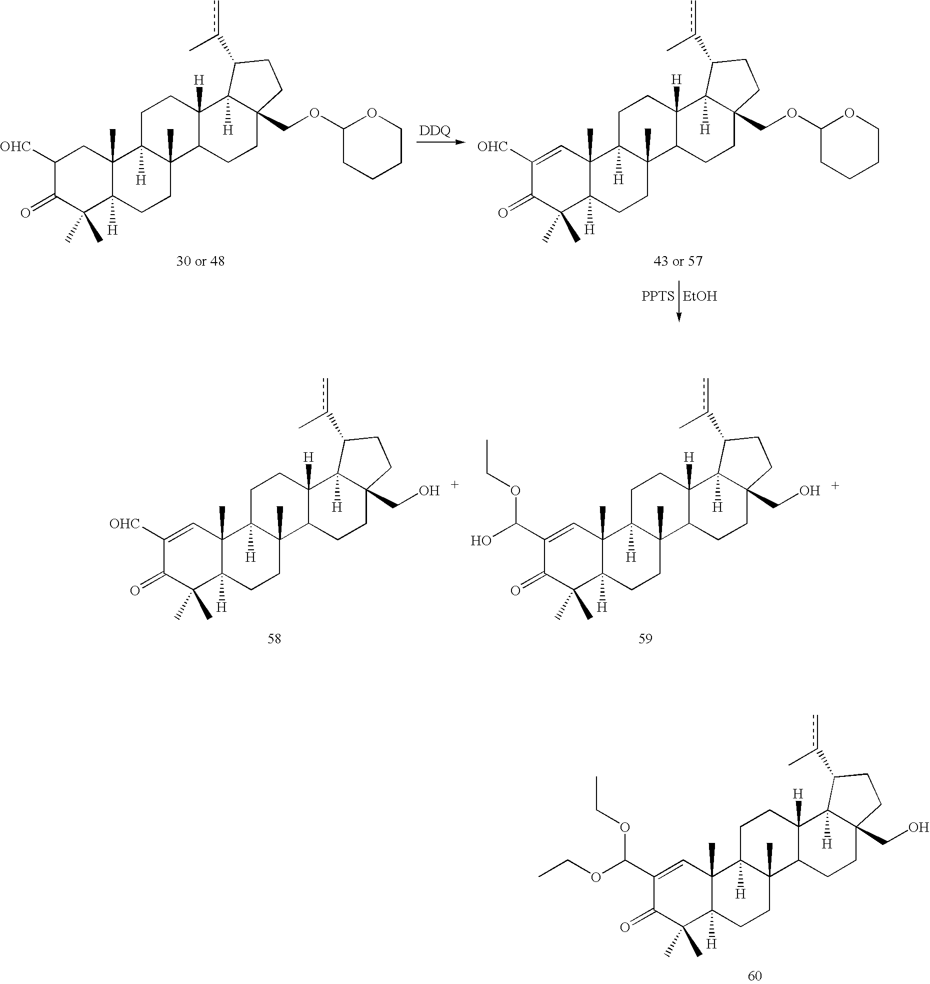

Further access to different C-2 substituted enone analogues of lupne-type pentacyclic triterpenoids was conceived by regio- and stereoselective 1,2-epoxidation of the A ring modified enone. Nucleophilic opening of the epoxide at the C-2 position and spontaneous dehydration of the resulting alkoxy or halohydrins furnished such C-2 substituted enones.

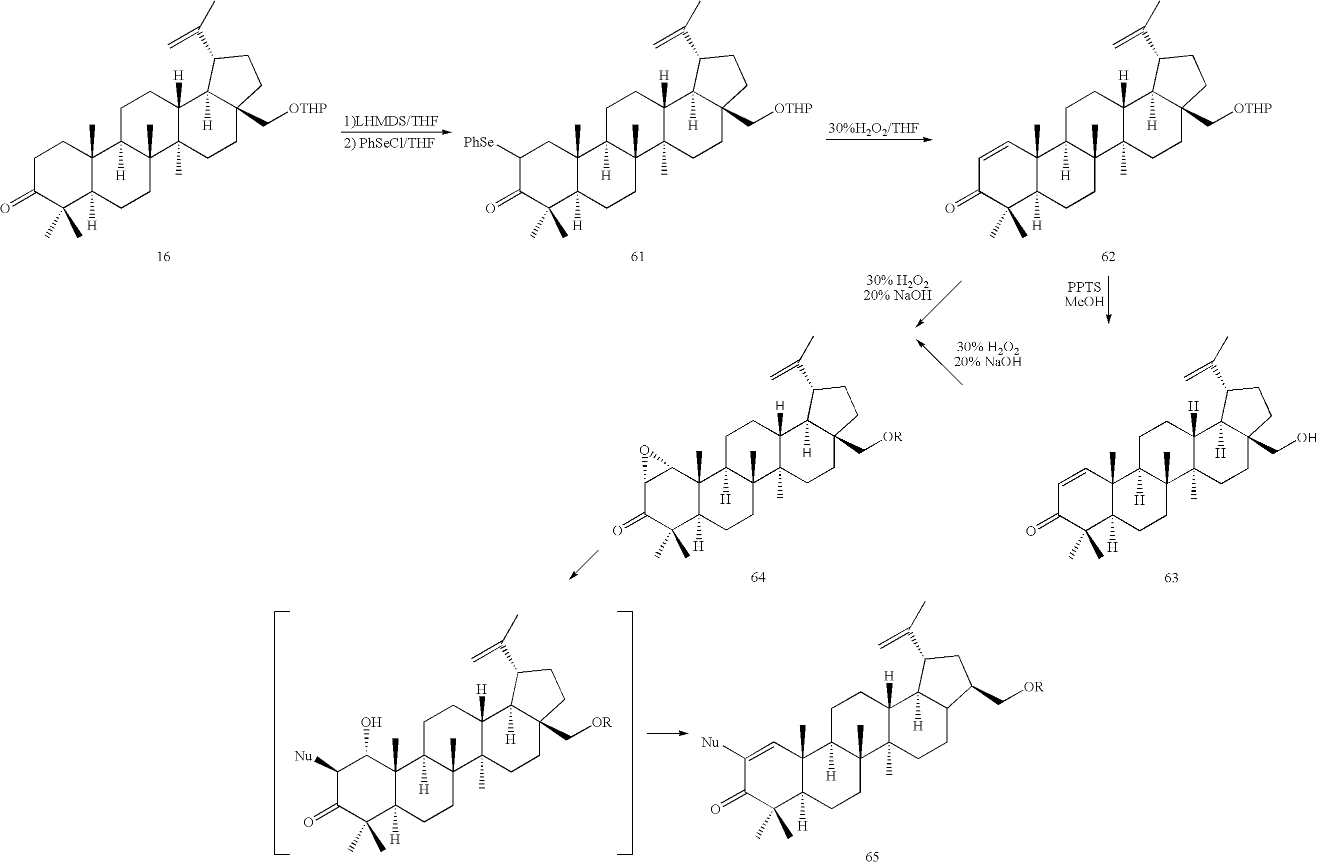

A series of new molecules including 28-aza and 3-aza analogues of betulinic acid and betulin were synthesized. For preparation of 28-aza analogues, betulin (2) was oxidized with CrO₃.pyridine complex and the resulting keto aldehyde 66 was subjected to reductive amination, in the presence of sodium cyanoborohydride, with glycine methyl ester hydrochloride, ethanolamine, and 2-chloroethylamine to provide 28-aza analogues 67, 68 and 69 respectively. The ketone moiety at the C-3 position was also reduced in all reactions. The resulting 3-hydroxy groups in 67, 68 and 69 are all assumed to have the 0 configuration by ¹H NMR spectral comparison to betulin. Hydrolysis of 67 to the amino acid 68 was achieved with KOH in THF and H₂O.

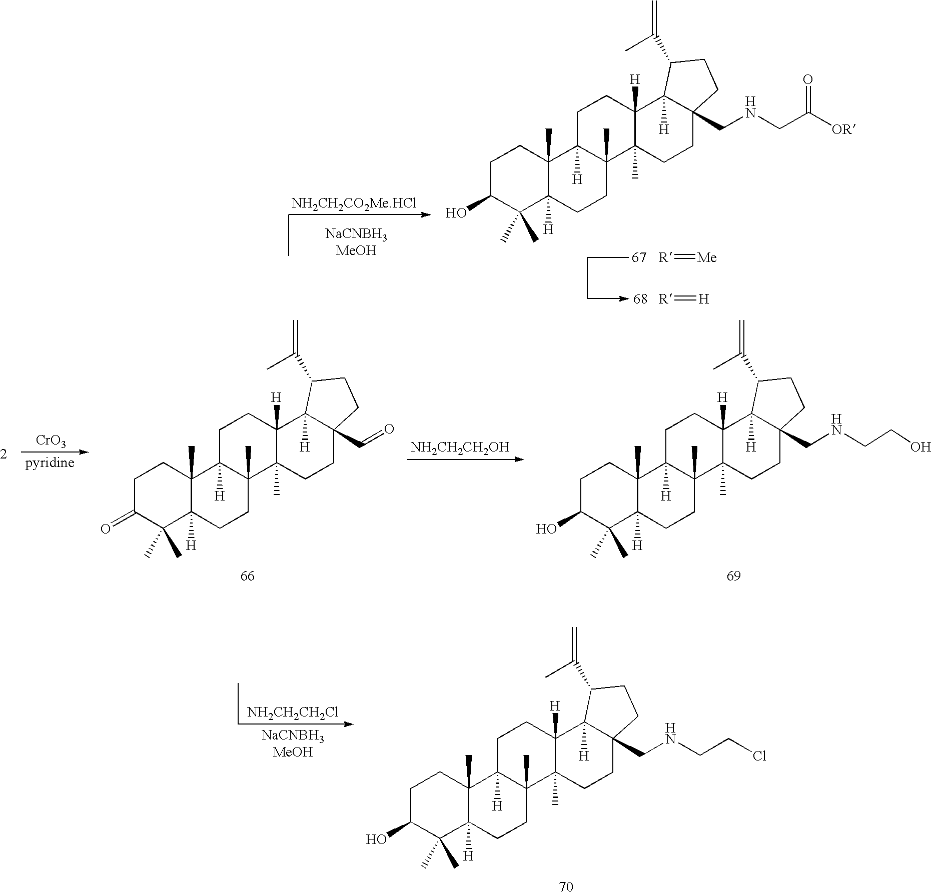

Oxidation of 3-acetylbetulin 7 followed by reductive amination of the resulting acetyl aldehyde with either glycine methyl ester hydrochloride or ethanolamine provided the new 28-aza analogues 72 and 73 respectively.

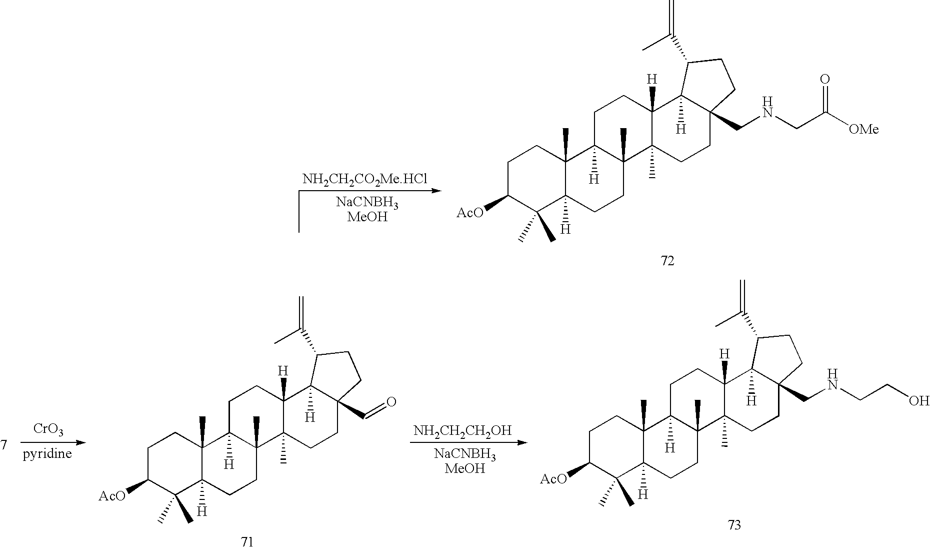

For the synthesis of 3-aza analogues, keto alcohol 17 or betulonic acid 3 successively underwent reductive amination in the presence of sodium cyanoborohydride to furnish amine compounds such as 74-82.

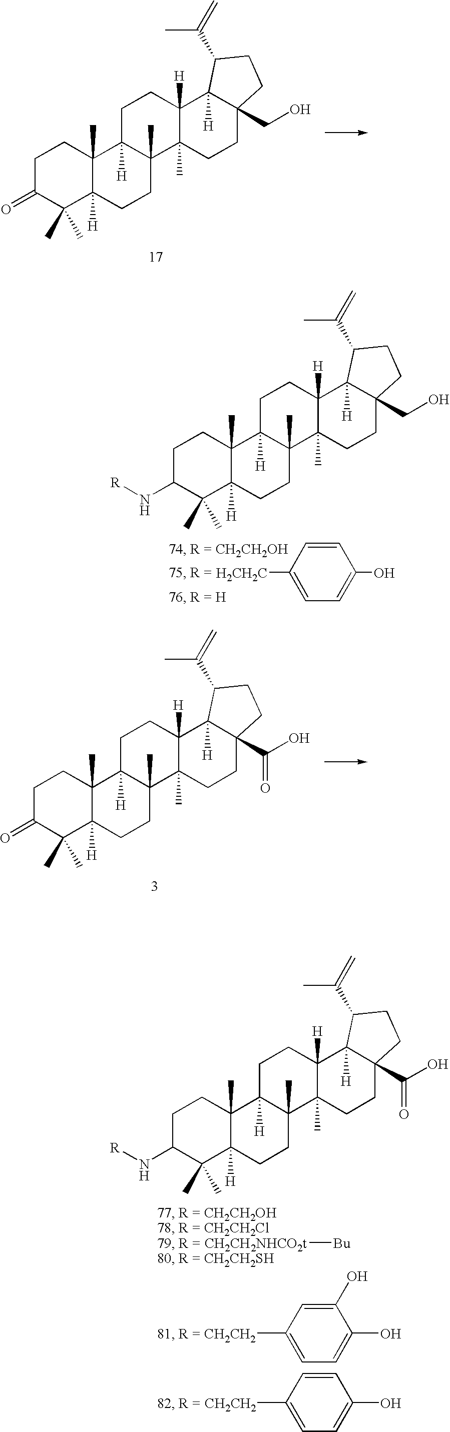

Reductive amination of betulonic acid (3) with ammonium acetate provided the corresponding 3-amino-betulinic acid 83, hydrogenation of which gave access to 20,29-dihydro-3-amino-betulinic acid (86). Further reductive amination of 83 and 86 with aldehyde or ketone compounds in the presence of NaCNBH₃ provided an additional route for the synthesis of substituted amino derivatives such as 84 and 85.

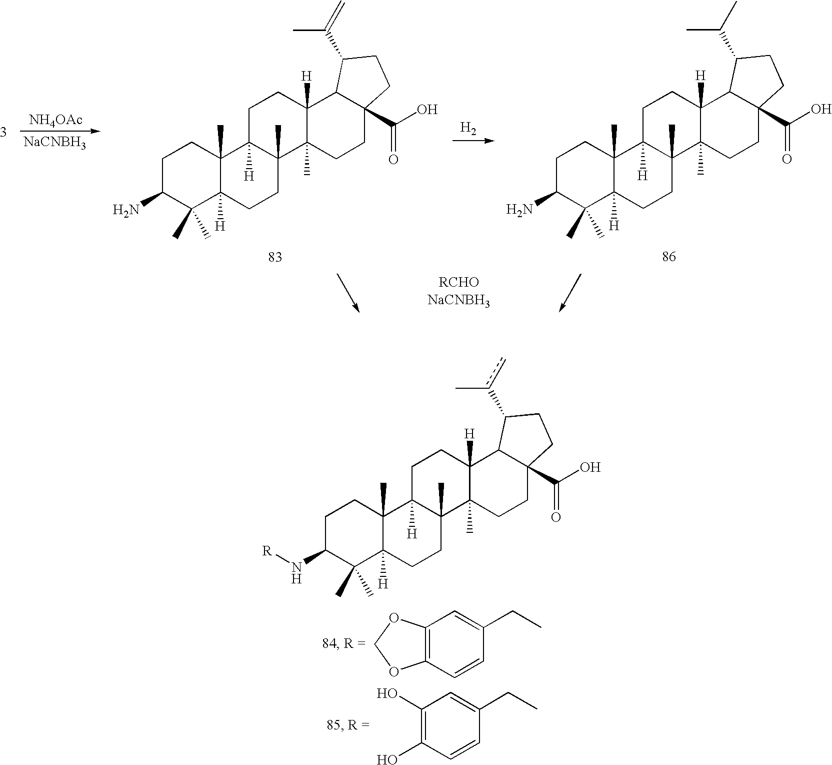

### EXPERIMENTAL

### Example 1

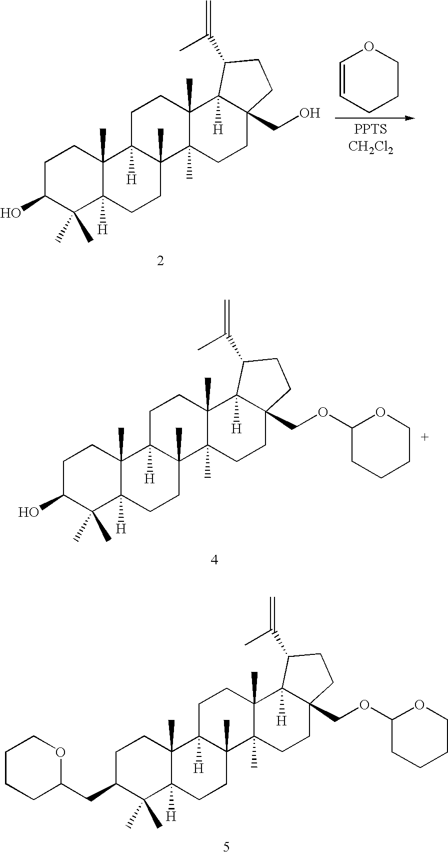

To a suspension of betulin (2) (4.5 g, 10.16 mmol) in anhydrous CH2Cl2 (150 mL), while stirring at rt under N₂, was added 3,4-dihydro-2H-pyran (0.94 g, 11.18 mmol) dropwise. Thereafter pyridinium p-tolune sulfonate (PPTS) (0.3 g, 1.20 mmol) was added all at once. The reaction was allowed to proceed at rt under N₂ for 4 days and monitored by TLC analysis. The reaction mixture was then quenched with saturated NaHCO₃ (50 mL). The organic layer was sepaated and washed with H₂ O (50 mL), dried over Na₂SO₄ and concentrated under reduced pressure to a crude yellow solid. Purification by SiO₂ column chromatography with gradient elution (5-20% EtOAc/hexane) afforded 2.8 g (52% yield) of 4 as a mixture of diastereomers (m.p. 135-140° C.) and 2.6 g (42% yield) of 5 as a mixture of diastereomers (m.p. 144-154° C.). Compound 4: ¹H NMR (400 MHz, CDCl₃) δ 0.6-2.1 (m), 0.76 (s), 0.82 (s), 0.96 (s), 0.97 (s), 1.01 (s), 1.03 (s), 1.68 (s), 2.43 (m), 2.98 (d, J=9.6 Hz), 3.18 (dd, J=5.2, 11.2 Hz), 3.37 (d, J=9.6 Hz), 3.52 (m), 3.86 (m), 3.92 (d, J=9.2 Hz ), 4.57 (m), 4.67 (s). Compound 5: ¹H NMR (400 MHz, CDCl₃) δ 0.6-2.1 (m), 0.77 (s), 0.78 (s), 0.83 (s), 0.88 (s), 0.89 (s), 0.95 (s), 0.96 (s), 1.012 (s), 1.015 (s), 1.02 (s), 1.68 (s), 2.43(m), 2.98 (d, J=9.6 Hz), 3.02 (dd, J=4.4, 11.6 Hz), 3.20 (dd, J=4.0, 11.6 Hz), 3.37 (d, J=9.6 Hz), 3.47 (m), 3.88 (m), 4.57 (m), 4.67 (s), 4.72 (m).

### Example 2

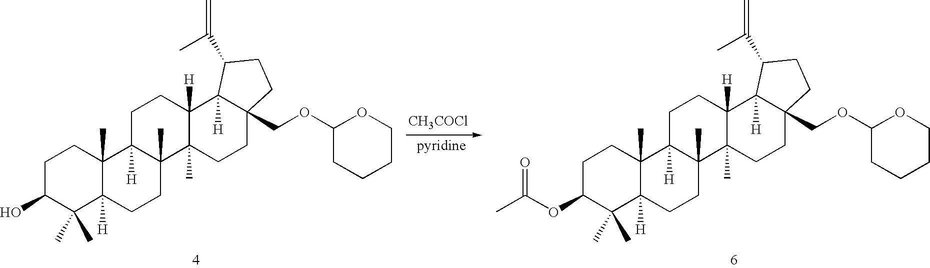

To a solution of 4 (1.0 g, 1.89 mmol) in pyridine (20 mL), while stirring at rt under N₂, was added AcCl (0.27 mL, 3.79 mmol) dropwise. The solution turned cloudy and yellow while precipitates were formed. After stirring for 16 h, pyridine was removed under vacuum. The crude residue obtained was partitioned between EtOAc (30 mL) and water (30 mL). The organic layer was separated, washed with H₂ O (30 mL) and brine (2×30 mL), dried over Na₂SO₄ and concentrated under reduced pressure. The orange/red crude residue was purified by gradient silica gel column chromatography (5-10% EtOAc/hexane) to provid 0.8 g (74% yield) of 6 as a mixture of diastereomers (m.p. 146-151° C). ¹H NMR (400 MHz, CDCl₃) δ 0.7-2.1 (m), 0.83 (s), 0.84 (s), 0.96 (s), 1.01 (s), 1.03 (s), 1.68 (s), 2.04 (s), 2.43(m), 2.98 (d, J=9.6 Hz), 3.37 (d, J=9.2 Hz), 3.50 (m), 3.84 (m), 3.92 (d, J=9.2 Hz), 4.47 (m), 4.57 (m), 4.67 (s).

### Example 3

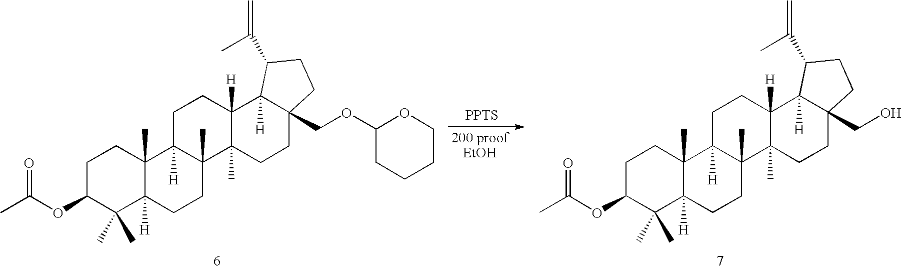

To a solution of 6 (300 mg, 0.53 mmol) in EtOH (20 mL) and CH₂Cl₂ (2 mL), while stirring at rt, was added PPTS (265 mg, 1.06 mmol). Afterr 6 days, p-toluenesulfonic acid monohydrate (PTSA.H₂O) (25 mg, 0.13 mmol) was added and stirring continued for another 24 h. The reaction mixture was then concentrated under reduced pressure. The crude residue obtained was dissolved in CH₂Cl₂ (30 mL) and washed with sat. NaHCO₃ (30 mL). The organic layer was separated, dried over Na₂SO₄ and concentrated under vacuum. Purification by SiO₂ column chromatography with gradient elution (5-20% EtOAc/hexane) afforded 243 mg (92% yield) of 7 (m.p. 259-268° C.). ¹H NMR (400 MHz, CDCl₃) δ 0.7-2.1 (m, 25 H), 0.82 (s, 3H), 0.84 (s, 6H), 0.96 (s, 3H), 1.01 (s, 3H), 1.69 (s, 3H), 2.03 (s, 3H), 2.37 (m, 1H), 3.33 (d, J=11.2 Hz, 1H), 3.78 (d, J=10.4 Hz, 1H), 4.46 (m, 1H), 4.58 (d, J=1.6 Hz, 1H), 4.67 (d, J=2.4 Hz, 1H). ¹³C NMR (125.6 MHz, CDCl₃) δ 14.7, 15.9, 16.1, 16.4, 18.1, 19.0, 20.8, 21.3, 23.6, 25.1, 27.0, 27.9, 29.1, 29.7, 33.9, 34.1, 37.0, 37.2, 37.7, 38.3, 40.9, 42.7, 47.7, 47.8, 48.7, 50.2, 55.3, 60.5, 80.9, 109.7,150.4,171.0.

### Example 4

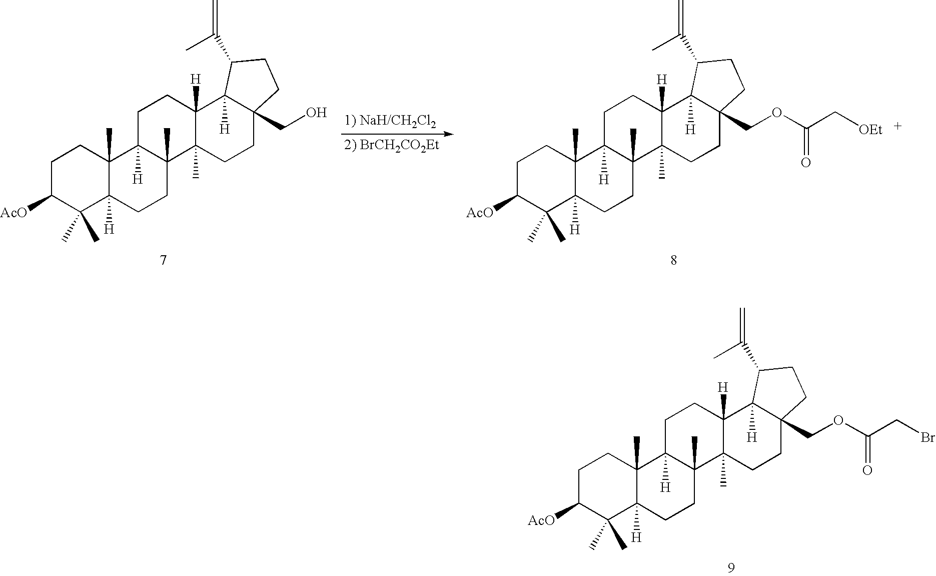

To a suspension of NaH (198 mg of 60% dispersion in mineral oil, 4.95 mmol) in anhydrous CH₂Cl₂ (2 mL) while stirring at rt under N₂, was added a solution of 7 (100 mg, 0.21 mmol) in anhydrous CH₂Cl₂ (1 mL) dropwise. After 0.5 h, a solution of ethyl bromoacetate (35 mg, 0.21 mmol) in anhydrous CH₂Cl₂ (1 mL) was added dropwise. The resulting mixture was stirred at rt for 16 h whereupon another equivalent of ethyl bromoacetate (35 mg, 0.21 mmol) was added. The reaction mixture was stirred for another 4 h and then quenched with H₂ O (1 mL). The mixture was poured in a mixture of EtOAc (2 mL) and brine (2 mL) while stirring. The organic layer was separated, dried over Na₂SO₄ and concentrated under reduced pressure to an oily residue, which was further purified by SiO₂ column chromatography with gradient elution (0-20% EtOAc/hexane) to yield 37 mg (31% yield) of 8 (m.p. 94-97° C.) and 65 mg (58% yield) of 9 (m.p. 97-100° C.). Compound 8: ¹H NMR (500 MHz, CDCl₃) δ 0.8-2.1 (m, 24 H), 0.83 (s, 3H), 0.844 (s, 3H), 0.848 (s, 3H), 0.96 (s, 3H), 1.03 (s, 3H), 1.26 (t, J=8 Hz, 3H), 1.68 (s, 3H), 2.04 (s, 3H), 2.44 (m, 1H), 3.60 (dq, J=6.8, 1.2 Hz, 2H), 3.93 (d, J=11.2 Hz, 1H), 4.09 (s, 2H), 4.38 (d, J=11.2 Hz, 1H), 4.46 (m, 1H), 4.59 (m, 1H), 4.69 (d, J=2.0 Hz, 1H). ¹³C NMR (125.6 MHz, CDCl₃) δ 14.7, 15.0, 16.0, 16.1, 16.4, 18.1, 19.0, 20.7, 21.3, 23.6, 25.1, 27.0, 27.9, 29.5, 29.7, 34.1, 34.5, 37.0, 37.6, 37.7, 38.3, 40.8, 42.7, 46.4, 47.7, 48.8, 50.2, 55.3, 63.1, 67.2, 68.0, 80.9, 110.0,150.0, 171.02, 171.06. Compound 9: ¹H NMR (400 MHz, CDCl₃) δ 0.7-2.1 (m, 25 H), 0.83 (s, 3H), 0.84 (s, 3H), 0.85 (s, 3H), 0.97(s, 3H), 1.03 (s, 3H), 1.68 (s, 3H), 2.04 (s, 3H), 2.42 (m, 1H), 3.86 (s, 2H), 3.95 (d, J=10.8 Hz, 1H), 4.38 (dd, J=1.2, 10.8 Hz, 1H), 4.47 (dd, J=5.2, 10 Hz, 1H), 4.59 (dd, J=1.6, 2.0 Hz, 1H), 4.69 (d, J=2.0 Hz, 1H). ¹³C NMR (100.5 MHz, CDCl₃) δ 14.7, 16.0, 16.1, 16.4, 18.1, 19.0, 20.7, 21.3, 23.6, 25.1, 25.9, 27.0, 27.9, 29.5, 29.6, 34.1, 34.4, 37.0, 37.6, 37.7, 38.3, 40.8, 42.7, 46.5, 47.7, 48.8, 50.2, 55.3, 64.8, 80.9, 110.0,149.9, 167.6, 171.0.

### Example 5

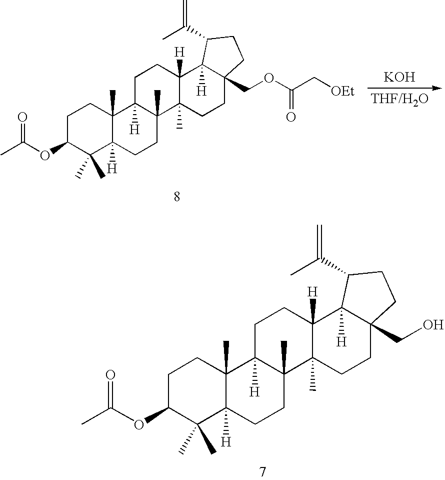

To a solution of 8 (14 mg, 0.025 mmol) in THF (0.5 mL), while stirring at rt, was added dropwise a solution of KOH (85%) (16.2 mg, 0.25 mmol) in distilled H₂ O (0.5 mL). The resulting heterogeneous mixture was stirred at rt for 48 h, acidified to pH ˜2 (10 drops of 1N HCl) and extracted with CH₂Cl₂ (3 mL). The organic layer was separated, dried over Na₂SO₄, concentrated under reduced pressure. The crude product was dissolved in CH₂Cl₂ and filtered through a short silica gel column path to afford 7 (11.1 mg, 90% yield) as a white solid.

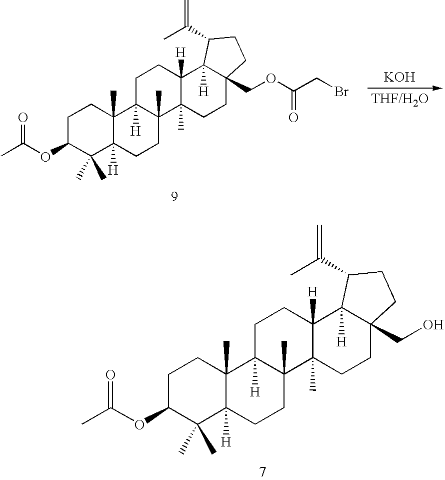

To a solution of 9 (15 mg, 0.028 mmol) in THF (0.5 mL), while stirring at rt, was added dropwise a solution of KOH (85%) (18.2 mg, 0.28 mmol) in distilled H₂ O (0.5 mL). The resulting heterogeneous mixture was stirred at rt for 48 h, acidified to pH ˜2 (10 drops of 1N HCl) and extracted with CH₂Cl₂ (3 mL). The organic layer was separated and dried over Na₂SO₄, concentrated under reduced pressure. The crude product was dissolved in CH₂Cl₂ and filtered through a short silica gel column path to afford 7 (10.7 mg, 77% yield) as a white solid.

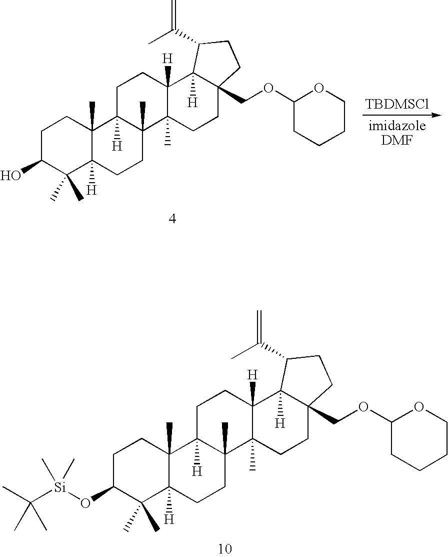

To a solution of 4 (1.0 g, 1.89 mmol) in DMF (25 mL), while stirring at rt under N₂, were added imidazole (0.39 g, 5.69 mmol) and TBDMSCl (0.57 g, 3.79 mmol) in succession. The resulting solution turned cloudy in 0.5 h and a precipitate began to form. The reaction mixture was stirred at rt for 48 h, whereupon H₂ O was added (200 mL). The white solids formed were collected through filtration, washed with H₂ O (2×100 mL) and dried under reduced pressure overnight to provide 1.09 g (90% yield) of 10 (m.p. 95-100° C.). ¹H NMR (500 MHz, CDCl₃) δ 0.026 (s), 0.6-2.1 (m), 0.72 (s), 0.81 (s), 0.88 (s), 0.96(s), 1.01 (s), 1.02 (s), 1.68 (s), 2.44(m), 2.98 (d, J=9 Hz), 3.15 (dd, J=4.5, 11 Hz) 3.37(d, J=9 Hz), 3.52 (m), 3.84 (m), 3.92 (d, J=10 Hz), 4.57 (m), 4.67 (s).

### Example 6

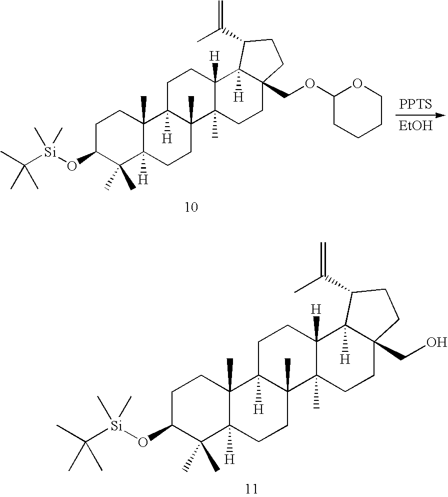

To a solution of 10 (300 mg, 0.52 mmol) in EtOH (20 mL) and CH₂Cl₂ (2 mL), while stirring at rt, was added PPTS (261 mg, 1.04 mmol). After 5 days, PTSA.H₂O (50 mg, 0.26 mmol) was added and stirring continued for another 24 h whereupon the reaction mixture was concentrated under reduced pressure. The residue was dissolved in CH₂Cl₂ (30 mL) and washed with saturated NaHCO₃ (30 mL) and H₂O (2×30 mL). The organic layer was separated, dried over Na₂SO₄, concentrated under reduced pressure and purified by SiO₂ column chromatography eluting with a gradient (5-20% EtOAc/hexane) to afford 243 mg (92% yield) of 11 (m.p. 127-130° C.). ¹H NMR (400 MHz, CDCl₃) δ 0.03 (s, 6H), 0.6-2.1 (m, 25 H), 0.72 (s, 3H), 0.82 (s, 3H), 0.88 (s, 12H), 0.97 (s, 3H), 1.01 (s, 3H), 1.68 (s, 3H), 2.39 (m, 1H), 3.15 (dd, J=4.4, 10.8 Hz, 1H), 3.33 (d, J=10.8 Hz, 1H), 3.80 (d, J=9.6 Hz, 1H), 4.58 (m, 1H), 4.68 (d, J=2.0 Hz, 1H).

### Example 7

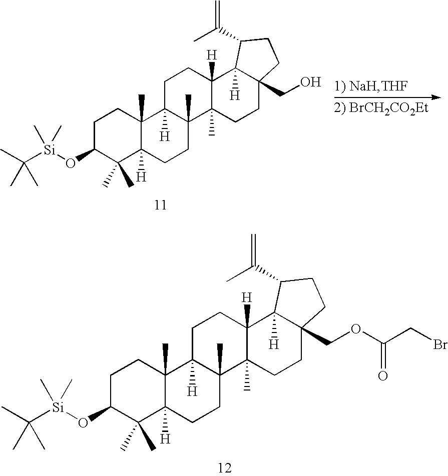

To a solution of 11 (113 mg, 0.20 mmol) in anhydrous THF (3 mL), while stirring at rt under N₂, was added NaH (16 mg of 60% dispersion in mineral oil, 0.40 mmol) portionwise. After 0.5 h, ethyl 2-bromoacetate (68 mg, 0.40 mmol) was added dropwise. The resulting suspension was stirred at rt for 72 h, quenched with H₂O (10 drops) and partitioned between EtOAc (2 mL) and brine (2 mL). The organic layer was separated, dried over Na₂SO₄ and concentrated under reduced pressure to an orange residue, which was purification by SiO₂ column chromatography using gradient elution (pure CH₂Cl₂ and then 0-50% EtOAc/hexane) to provide 92 mg (71% yield) of 12 (m.p. 177° C.). ¹H NMR (400 MHz, CDCl₃) δ 0.027 (s, 6H) 0.6-2.1 (m, 25H), 0.72 (s, 3H), 0.82 (s, 3H), 0.88 (s, 12H), 0.97 (s, 3H), 1.02 (s, 3H), 1.68 (s, 3H), 2.43 (m, 1H), 3.15 (dd, J=4.8, 11.2 Hz, 1H), 3.86 (s, 2H), 3.96 (d, J=11.2 Hz, 1H), 4.37 (d, J=12 Hz, 1H), 4.60 (m, 1H), 4.69 (d, J=2.0 Hz, 1H); ¹³C NMR (400 MHz, CDCl₃) δ−4.9,−3.7, 14.7, 15.8, 16.0, 16.1, 18.1, 18.4, 19.1, 20.7, 25.2, 25.92, 25.98, 27.0, 27.8, 28.4, 29.5, 29.6, 34.2, 34.4, 37.0, 37.6, 38.6, 39.4, 40.8, 42.6, 46.5, 47.6, 48.8, 50.3, 55.3, 64.8, 79.4, 109.9,149.9, 167.6.

### Example 8

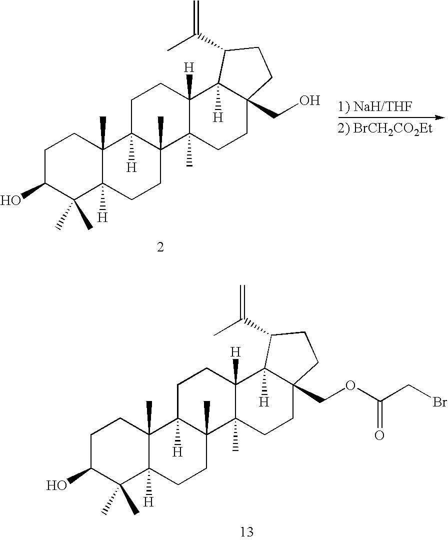

To a suspension of NaH (13 mg, 0.56 mmol) in THF (5 mL), while stirring at rt, was added a solution of 2 (betulin) (100 mg, 0.23 mmol) in THF (mL). After 0.5 h at rt, ethyl 2-bromoacetate (110 mg, 0.68 mmol) was added and the resulting mixture was allowed to stir at rt for 5 days, whereupon the reactin was quenched with water (5 mL). The mixture was extracted with EtOAc (15 mL) and the organic layer washed with 0.5 N HCl (10 mL), dried over Na₂SO₄ and concentrated under reduced pressure. The crude product obtained was purified by SiO₂ column chromatography eluting with 15:1 hexane/ethyl acetate to furnish 40 mg of 13 (36% yield) as solid (m.p. 170° C.). ¹H NMR (400 HMz, CDCl₃) δ 0.76 (s, 3H), 0.82 (s, 3H), 1.68 (s, 3H), 0.6-2.1 (m, 35 H), 2.3-2.5 (m, 1H), 3.16,3.17, 3.19, 3.2 (dd, J=4.8 Hz, j=5.2 Hz, 1H), 3.94, 3.96 (d, J=9.6 Hz, 1H) 3.78-4.28 (m, 2H), 4.36, 4.37, 4.393, 4.398 (dd, J=2.0 Hz, J=2.0 Hz, 1H), 4.58-4.60 (m, 1H), 4.691-4.698 (m, 1H); ¹³C NMR (400MHz, CDCl₃) δ 14.7, 15.3, 16.01, 16.08, 18.2, 19.1, 20.7, 25.1, 25.9, 27.0, 27.3, 27.9, 29.5, 29.6, 34.1, 34.4, 37.1, 37.6, 38.6, 38.8, 40.8, 42.7, 46.5, 47.6, 48.8, 50.3, 55.2, 64.8, 78.9, 109.9, 149.9, 167.6.

### Example 9

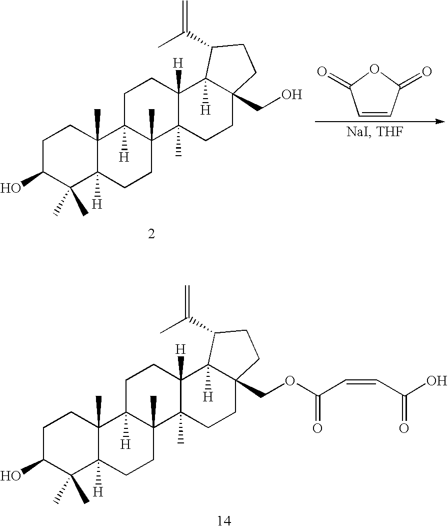

To a suspension of NaH (27 mg, 1.12 mmol) in THF (5 mL), while stirring at rt under N₂, was added 2 (betulin) (200 mg, 0.45 mmol). After 15 min, maleic anhydride (130 mg, 1.35 mmol) and NaI (70 mg, 0.45 mmol) were added in succession. The suspension was stirred overnight, whereupon it was cooled to 0° C. and quenched with dropwise addition of water (5 mL). The resulting mixture was extracted with ethyl acetate (3×5 mL) and the combined organic layers were washed successively with 0.5 N HCl (10 mL), 10% NaHCO₃ (10 mL), brine (10 mL) and water (10 mL). was After being dried over Na₂SO₄ and concentrated under vacuum, the crude material thus obtained was purified by SiO₂ column chromatography (5% MeOH/CH₂Cl₂) to yield 100 mg (44% yield) of 14 (m.p. 233° C). ¹H NMR (400 HMz, CDCl₃): δ 0.60-1.98 (m, 24 H), 0.65 (s, 3H), 0.76 (s, 3H), 0.87 (s, 3H), 0.93 (s, 3H), 0.99 (s, 3H), 1.64 (s, 3H), 2.46 (m, 1H), 2.97 (m, 1H), 2.98-3.6 (broad, 1H), 3.76, (d, J=11.2 Hz, 1H), 4.20 (broad, 1H), 4.29 (d, J =10.4 Hz, 1H), 4.56 (s, 1H), 4.70 (d, J=2 Hz, 1H), 6.05 (d, J=12 Hz, 1H), 6.35 (d, J=12 Hz, 1H).

### Example 10

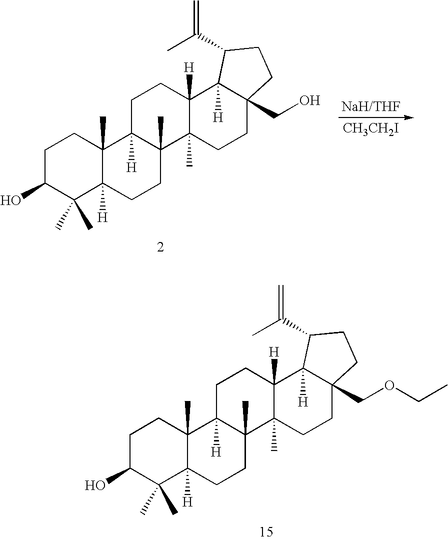

To a suspension of NaH (330 mg, 13.71 mmol) in THF (5 mL), while stirring at rt under N₂, was added 2 (betulin) (200 mg, 0.45 mmol). After 0.5h, iodoethane (0.11 mL, 1.37 mmol) was added and the resultant reaction mixture was stirred at rt for 3 days, whereupon water (5 mL) was added carefully. The mixture was extracted with EtOAc (3×10 mL). The organic layers were combined and washed successively with 0.5 N HCl (10 mL), 10% NaHCO₃ (10 mL), brine (10 mL) and water (10 mL). After being dried over Na₂SO₄ and concentrated under reduced pressure, the crude product was purified by SiO₂ column chromatography (8:1 Hexane/EtOAc) to afford 34 mg (16% yield) of 15 (m.p. 114-117° C.). ¹H NMR (500 HMz, CDCl₃) δ 0.76 (s, 3H), 0.82 (s, 3H), 0.97 (s, 3H), 1.03 (s, 3H), 1.33 (s, 3H), 1.67 (s,3H), 1.67 (s,3H), 0.76-2.04 (m, 29H), 2.37-2.43 (m, 1H), 3.08 (d, J=10 Hz, 1H), 3.19 (dd, J=5, 5 Hz, 1H), 3.45-3.52 (m, 2H), 4.57 (m, 1H), 4.67 (d, J=2 Hz, 1H).

### Example 11

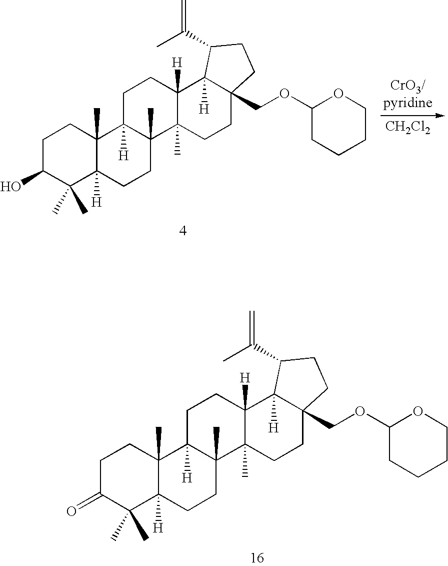

To a solution of pyridine (0.97 mL, 12.0 mmol) in CH₂Cl₂ (20 mL), while stirring at rt under N₂, was added CrO₃ (600 mg, 6.0 mmol). The resulting dark brown suspension was stirred at rt for 1 h, whereupon it was cooled to 0° C. and into which was added dropwise a solution of 4 (526.9 mg, 1.0 mmol) in CH₂Cl₂ (5 mL). The suspension was stirred at 0° C. for an additional hour and filtered through a short column of silica gel eluting with CH₂Cl₂/hexane (1:1) and 10% EtOAc/hexane. Fractions containing the product were combined and concentrated under reduced pressure to afford 304 mg (58% yield) of 16 as a mixture of diastereomers (m.p. 85-90° C.). 1H NMR (400 MHz, CDCl₃) δ 0.8-2.1 (m), 0.92 (s), 0.98 (s), 1.02 (s), 1.05 (s), 1.07 (s), 1.68 (s), 2.44(m), 2.99 (d, J=9.6 Hz), 3.38 (d, J=9.2 Hz), 3.53 (m), 3.85 (m), 3.93 (d, J=8.4 Hz), 4.57 (m), 4.68 (s).

### Example 12

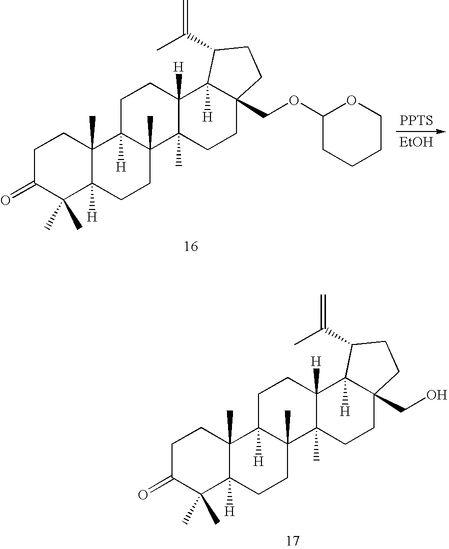

To a solution of 16 (224 mg, 0.43 mmol) in EtOH (3 mL), while stirring at rt, was added PPTS (214 mg, 0.85 mmol). After 2 days, PTSA.H₂O (25 mg, 0.13 mmol) was added and stirring was continued at rt for 3 days. The reaction mixture was concentrated under reduced pressure and the residue obtained was dissolved in CH₂Cl₂ (20 mL) and washed with saturated NaHCO₃ (20 mL) and H₂O (20 mL). After being dried over Na₂SO₄ and concentrated under reduced pressure, the residue was purified by SiO₂ column chromatography with gradient elution (0-50% EtOAc/hexane) to provide 105 mg (56% yield) of 17 as a white foam (m.p. 118-120° C.). ¹H NMR (400 MHz, CDCl₃) δ 0.8-2.1 (m, 24H), 0.92 (s, 3H), 0.99 (s, 3H), 1.02 (s, 3H), 1.06 (s, 3H), 1.07 (s, 3H), 1.68 (s, 3H), 2.43 (m, 2H), 3.35 (d, J=10.8 Hz, 1H), 3.80 (dd, J=1.2, 10.8 Hz, 1H), 4.58 (m, 1H), 4.68 (d, J=2.4 Hz, 1H). ¹³C NMR (400 MHz, CDCl₃) δ 14.6, 15.7, 15.9, 19.0, 19.6, 21.0, 25.1, 26.6, 27.0, 29.1, 29.7, 33.4, 33.9, 34.1, 36.8, 37.4, 39.5, 40.8, 42.7, 47.3, 47.7, 48.6, 49.7, 54.9, 60.5, 109.7, 150.3, 218.1.

### Example 13

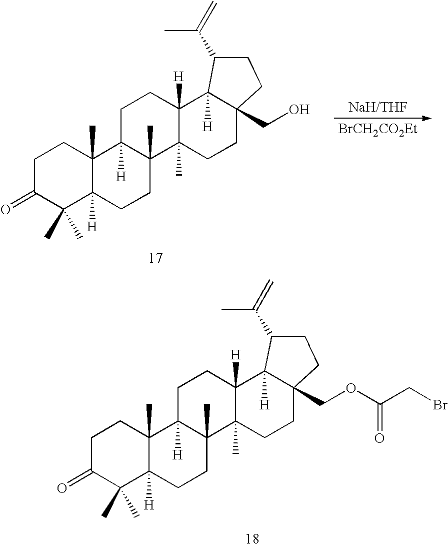

To a suspension of NaH (0.6 g, 6.8 mmol) in THF (5 mL), while stirring at rt under N₂, was added 17 (0.1 g, 0.22 mmol). After 0.5 h, ethyl 2-bromo acetate (0.075 mL, 0.68 mmol) was added dropwise. The resulting reaction mixture was stirred at rt overnight, whereupon it was quenched with water (5 mL). The reaction mixture was then extracted with EtOAc (3×10 mL) and the combined organic layers were washed successively with 0.5N HCl (5 mL), water (5 mL), 10% NaHCO₃ (5 mL) and brine (5 mL). After being dried over Na₂SO₄ and concentrated under reduced pressure, the crude material was purified by SiO₂ column chromatography (8:1 hexane/EtOAc) to afford 10.6 mg (10% yield) of 18 (m.p. 95-100° C.). ¹H NMR (500 MHz, CDCl₃) δ 0.93 (s, 3H), 0.99 (s, 3H), 1.02 (s, 3H), 1.07 (s, 6H), 1.68 (s, 3H), 0.93-2.0 (m, 23H), 2.46 (m, 3H), 3.86 (s, 2H), 3.97 (d, J=15 Hz, 1H), 4.40 (d, J=10 Hz, 1H), 4.60 (m, 1H), 4.70 (s, 1H).

### Example 14

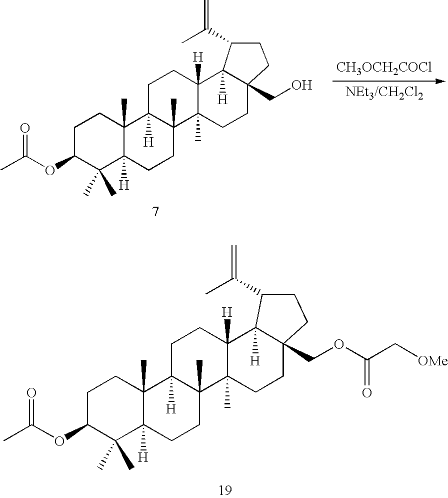

To a solution of 7 (44 mg, 0.09 mmol) and NE₃ (0.1 mL, 0.72 mmol) in CH₂Cl₂ (2 mL), while stirring at 0° C. under N₂, was added methoxyacetyl chloride (19 mg, 0.17 mmol) dropwise. The solution was warmed up to rt and stirring was continued for 24 h. The reaction mixture was then diluted with MeOH (1 mL) and EtOAc (10 mL). The organic layer was separated and washed with saturated NaHCO₃ (10 mL). After being dried over Na₂SO₄ and concentrated under reduced pressure, a clear oil was obtained which was further purified by silica gel column chromatography eluting with a gradient of 0-20% EtOAc/hexane to provide 33 mg (65% yield) of 19 (m.p. 133-137° C.). ¹H NMR (400 MHz, CDCl₃) δ 0.7-2.1 (m, 24 H), 0.83 (s, 3H), 0.844 (s, 3H), 0.849 (s, 3H), 0.96 (s, 3H), 1.03 (s, 3H), 1.68 (s, 3H), 2.04 (s, 3H), 2.43 (m, 1H), 3.46 (s, 3H), 3.94 (d, J=10.5 Hz, 1H), 4.05 (d of AB quartet, 2H), 4.38 (d, J=11 Hz, 1H), 4.46 (dd, J=6.5 and 10.5 Hz, 1H), 4.59 (m, 1H), 4.69 (d, J=1.5 Hz, 1H). ¹³C NMR (125.6 MHz, CDCl₃) δ 14.7, 16.0, 16.1, 16.4, 18.1, 19.0, 20.7, 21.3, 23.6, 25.1, 27.0, 27.9, 29.5, 29.7, 34.1, 34.5, 37.0, 37.6, 37.7, 38.3, 40.8, 42.6, 46.4, 47.6, 48.7, 50.2, 55.3, 59.4, 63.1, 69.8, 80.9, 109.9, 149.9, 170.7, 171.0.

### Example 15

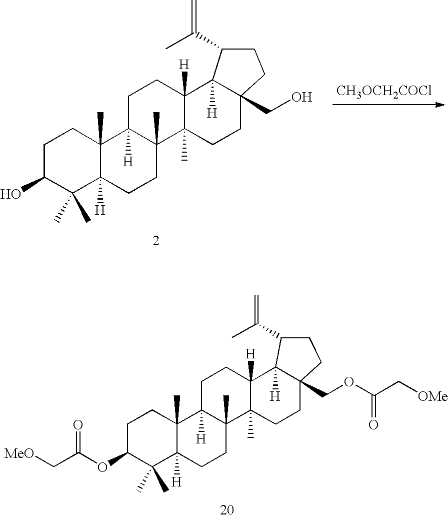

To a solution of betulin (2) (100 mg, 0.22 mmol) in pyridine (3 mL), while stirring at 0° C. under N₂, was added dropwise methoxyacetyl chloride (0.1 mL, 1.09 mmol). The solution was warmed up to rt and stirring was continued for 2 days. The reaction was hen quenched by careful addition of H₂O (10 mL) and the mixture extracted with EtOAc (20 mL). The organic layer was washed with 0.2 N HCl (10 mL) and sat. NaHCO₃ (10 mL), dried over Na₂SO₄ and concentrated under reduced pressure to a brown foam, which was purified by silica gel column chromatography eluting with a gradient of 0-15% EtOAc/hexane to provide 114 mg (86% yield) of 20 (m.p. 125-130° C.). ¹H NMR (400 MHz, CDCl₃) δ 0.7-2.1 (m, 24 H), 0.84 (s, 3H), 0.85 (s, 6H), 0.97 (s, 3H), 1.03 (s, 3H), 1.68 (s, 3H), 2.44(m, 1H), 3.45 (s, 3H), 3.46 (s, 3H), 3.94 (d, J=11 Hz, 1H), 4.01 (d of AB quartet, 2H), 4.05 (d of AB quartet, 2H), 4.38 (d, J=11 Hz, 1H), 4.59 (m, 2H), 4.69 (m, 11H).

### Example 16

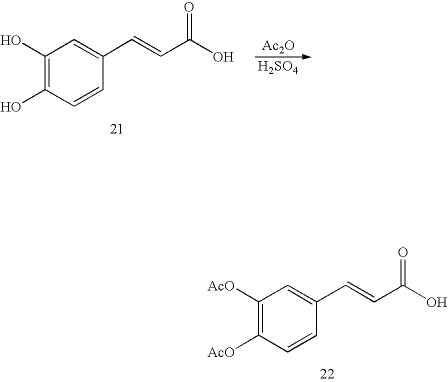

To a suspension of 21 (caffeic acid) (3 g, 19.98 mmol) in acetic anhydride (11.61 g, 113. 88 mmol), whilw stirring at rt, was added dropwise sulfuric acid (0.05 mL). The resulting mixture was heated at 60° C. for 1 h, whereupon it was cooled to rt and poured into ice water (100 mL). After stirring vigorously for 0.5 h, the off-white solids were collected through filtration and washed with water until the filtrate is neutral. The crude solid (4.02 g) was re-crystallized (EtOAc/hexane) to afford 2.9 g (65% yield) of 22 (m.p. 205-208° C.). ¹H NMR (500 MHz, CDCl₃) δ 2.29 (s, 6H), 6.55 (d, J=15 Hz, 1H), 7.32 (s, J=15 Hz, 1H), 7.62 (m, 3H), 12.46 (s, 1 H).

### Example 17

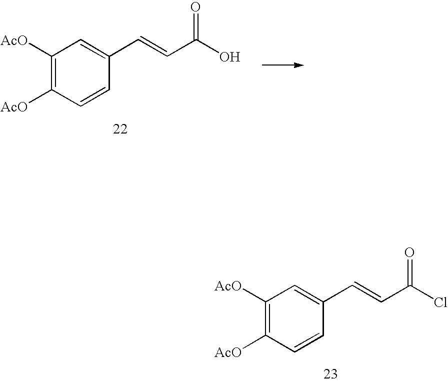

A solution of 22 (2.9 g, 10.97 mmol) in SOCl₂ (65.4 g, 549.7 mmol) and benzene (320 mL) was stirred at 80° C. for 3 h, whereupon the resulting solution was cooled to rt. Concentration under reduced pressure afforded 2.9 g (93% yield) of 23 (m.p. 86-95° C.). This material was used without further purification.

### Example 18

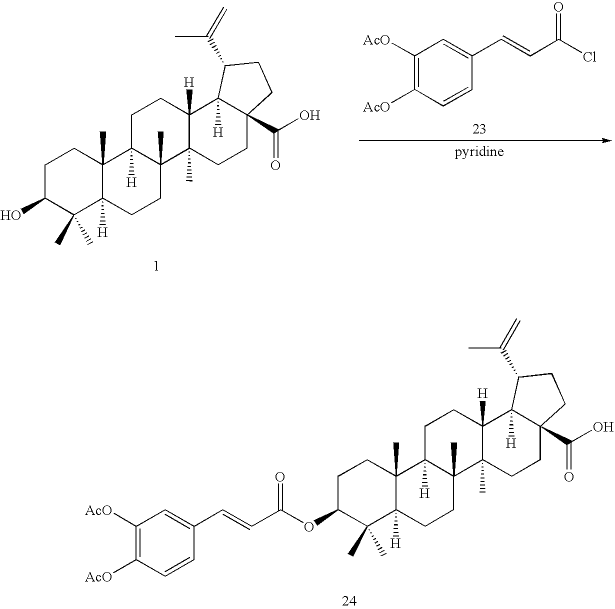

To a solution of betulinic acid (1) (0.2 g, 0.43 mmol) in pyridine (10 mL), while stirring at rt under N₂, was added neat 23 (0.60 g, 2.08 mmol) dropwise. After stirring for 8 days, pyridine was removed under reduced pressure and the resulting residue was diluted with EtOAc (10 mL) and washed with water (3×10 mL). The organic layer was dried over Na₂SO₄ and concentrated under reduced pressure. The crude material thus obtained was purified by SiO₂ column chromatography (0.5% MeOH/CH₂Cl₂) to give 30 mg (8% yield) of 24 (m.p. 209-211 OC). ¹H NMR (500 MHz, DMSO-d₆) δ 0.83 (s, 6H), 0.88 (s, 6H), 0.95 (s, 3H), 1.65 (s, 3H), 2.28 (s, 6H), 0.78-2.31(m, 24H), 2.95 (m, 1H), 4.56 (m, 1H), 4.56 (s, 1H), 4.69 (s, 1H), 6.64 (m, 1H), 7.32 (m, 1H), 7.72-7.59 (m, 3H), 12.07 (bs, 1H).

### Example 19

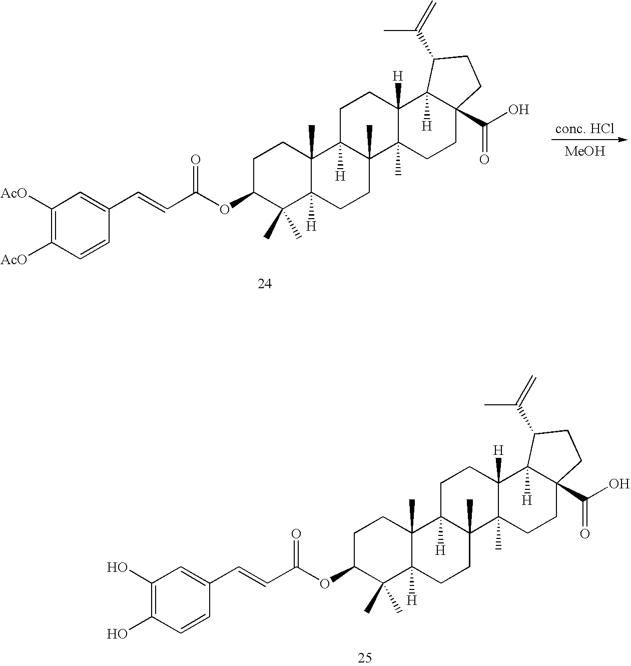

A solution of 24 (0.16 g, 0.22 mmol) in MeOH (3 mL) and THF (3 mL) was acidified by careful addition of conc. HCl (0.36 mL) and then heated at 60° C. for 15 min. The resulting solution was cooled to rt, diluted with water (5 mL) and extracted with EtOAC (2×2 mL). The combined organic layers were washed with brine (5 mL), dried over Na₂SO₄ and concentrated under reduced pressure. The crude solid was further purified by SiO₂ column chromatography (2% MeOH/CH₂Cl₂) to afford 47 mg (33% yield) of 25 (m.p. 290-294° C.). ¹H NMR (500 MHz, DMSO-d₆) δ 0.81 (s, 3H), 0.83 (s, 3H), 0.87 (s, 3H), 0.88 (s, 3H), 1.65 (s, 3H), 0.75-1.80 (m, 22H), 2.13 (m, 1H), 2.23 (m, 1H), 2.95 (m, 1H), 4.98 (m, 1H), 4.56 (s, 1H), 4.69 (s, 1H), 6.21 (s, 1H), 6.24 (s, 1H), 6.75 (d, J=10 Hz, 1H), 7.03 (m, 2H), 7.45 (d, J=15 Hz, 1H), 9.4 (bs, 1H) 12.15 (bs, 1H).

### Example 20

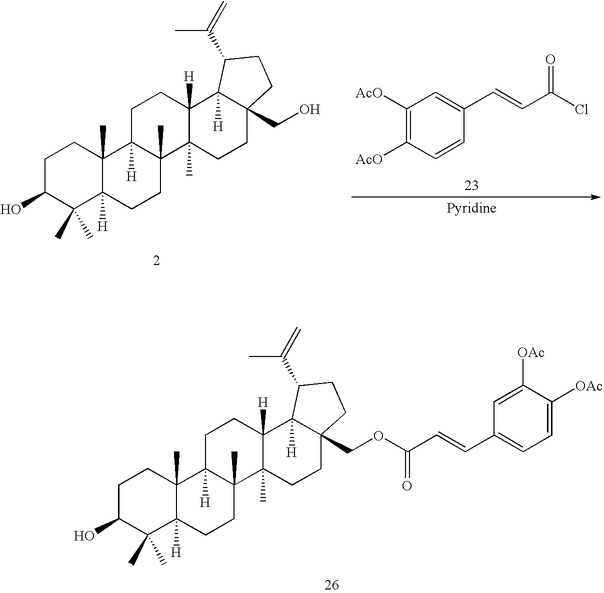

To a solution of betulin (2) (0.2 g, 0.45 mmol) in pyridine (10 mL), while stirring at rt under N₂, was added neat 23 (0.3 g, 1.04 mmol) dropwise. After 7 days, pyridine was removed by concentration under reduced pressure. The resulting residue was taken up in EtOAc (5 mL) and washed with water (3×5 mL). The organic layer was dried over Na₂SO₄ and concentrated under vacuum. The crude material was purified by SiO₂ column chromatography (4:1 hexane/EtOAc) to afford 79 mg (25% yield) of 26 (m.p. 214-216° C.). ¹H NMR (500 MHz, DMSO-d₆) δ 0.66 (s, 3H), 0.77 (s, 3H), 0.87 (s, 3H), 0.95 (s, 3H), 1.01 (s, 3H), 1.66 (s, 3H), 0.66-2.00 (m, 25H), 2.29 (s, 6H), 2.97 (m, 1H), 3.91 (d, J=10 Hz, 1H), 4.26 (d, J=5 Hz, 1H), 4.45 (d, J=10 Hz, 1H), 4.57 (s, 1H), 4.72 (s, 1H), 6.68 (m, 1H), 7.32 (d, J=5 Hz, 1H), 7.63-7.73 (m, 3H).

### Example 21

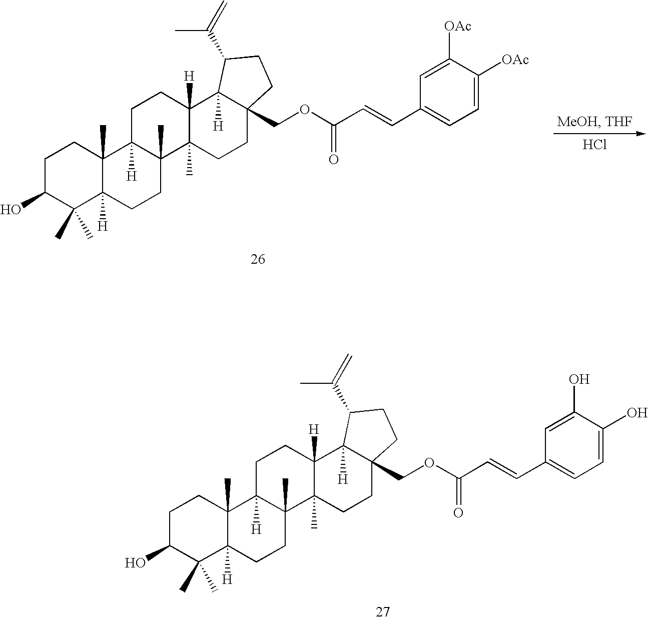

A solution of 26 (70 mg, 0.10 mmol) in MeOH (1.5 mL) and THF (1.5 mL) was acidified by careful addition of conc. HCl (0.18 mL) and heated at 60° C. for 15 min. The resulting solution was cooled down to rt, diluted with water (5 mL) and extracted with EtOAc (2×2 mL). The combined organic layers were washed with brine (5 mL), dried over Na₂SO₄) and concentrated under vacuum. The crude solid obtained was further purified by SiO₂ column chromatography (3:1 hexane/EtOAc) to afford 40 mg (65% yield) of 27 (m.p. 189-200° C.). ¹H NMR (500 MHz, DMSO-d₆) δ 0.65 (s, 3H), 0.77 (s,3H), 0.87 (s, 3H), 0.95 (s, 3H), 1.01 (s, 3H)1.66 (s, 3H), 0.63-2.00 (m, 25H), 2.97 (m, 1H), 3.33 (b, 3H), 3.87 (m, 1H), 4.42 (d, J=15 Hz, 1H), 4.57 (s, 1H), 4.71 (s, 1H), 6.28 (m, 1H), 6.76 (d, J=10 Hz, 1H), 6.99-7.04 (m, 2H), 7.49 (m, 1H).

### Example 22

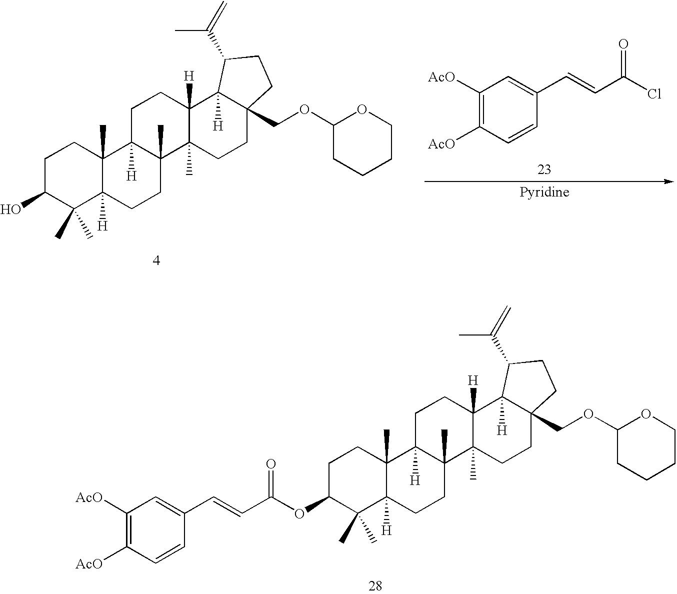

To a solution of 4 (0.5 g, 0.94 mmol) in pyridine (10 mL), while stirring at rt under N₂, was added neat 23 (0.32 g, 1.13 mmol) dropwise. After 2 days, pyridine was removed under reduced pressure. The resulting residue was taken up in EtOAc (6 mL) and washed with water (3×5mL). The organic layer was dried over Na₂SO₄ and concentrated under vacuum. The crude material was purified by SiO₂ column chromatography (4:1 hexane/EtOAc) to give 317 mg (48% yield) of 28 (m.p. 147-155° C.). ¹H NMR (500 MHz, DMSO-d₆) δ 0.83 (s, 6H), 0.88 (s, 3H), 0.97 (s, 3H), 0.99 (s, 3H),1.65 (s, 3H), 2.28 (m, 6H), 0.71-2.50 (m, 31H), 3.96 (m, 1H), 3.44 (m, 1H), 3.70-3.91 (m, 2H), 4.43-4.57 (m, 3H), 4.68-4.72 (m, 1H), 6.64 (m, 1H), 7.32 (m, 1H), 7.59-7.72 (m, 3H).

### Example 23

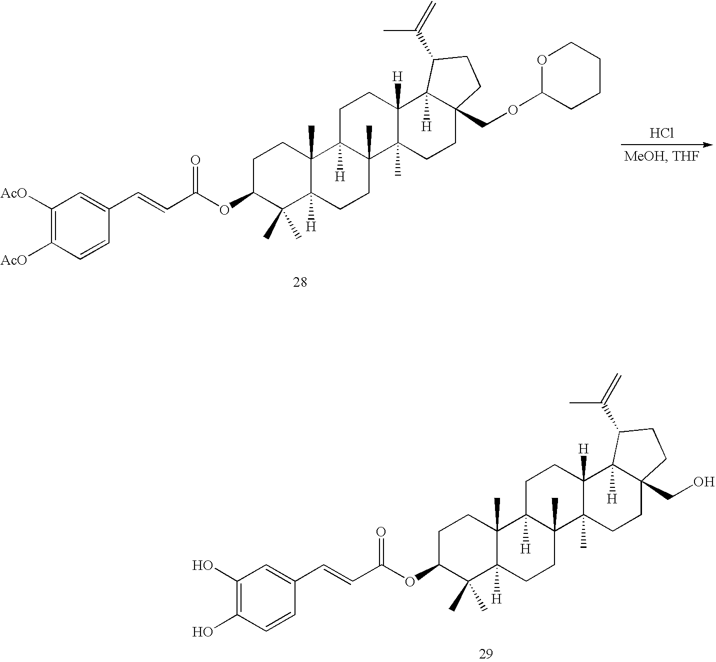

A solution of 28 (0.12 g, 0.17 mmol) in methanol (1.5 mL) and THF (1.5 mL) was acidified by careful addition of conc. HCl (0.18 mL) and heated at 60° C. for 15 min. The resulting solution was cooled down to rt, diluted with water (5 mL) and extracted with EtOAc (2×2 mL). The combined organic layers were washed with brine (5 mL), over Na₂SO₄ and concentrated under vacuum. The crude solid was further purified by SiO₂ column chromatography (3:1 hexane/EtOAc) to afford 66 mg (65% yield) of 29 (m.p. 207-216° C.). ¹H NMR (500 MHz, DMSO-d₆) δ 0.82 (s, 3H), 0.83 (s, 3H), 0.87 (s, 3H), 0.95 (s, 3H), 0.99 (s, 3H), 1.64 (s, 3H), 0.65-1.89 (m, 23H), 2.4 (m, 1H), 2.95-3.03 (m, 1H), 3.09 (d, J=15 Hz, 1H), 3.32 (b, 3H), 3.53 (d, J=10 Hz, 1H), 4.48 (m, 1H), 4.54 (m, 1H), 4.57 (m, 1H), 6.24 (m, 1H), 6.75 (d, J=15 Hz, 1H), 6.97-7.04 (m, 2H), 7.45 (m, 1H).

### Example 24

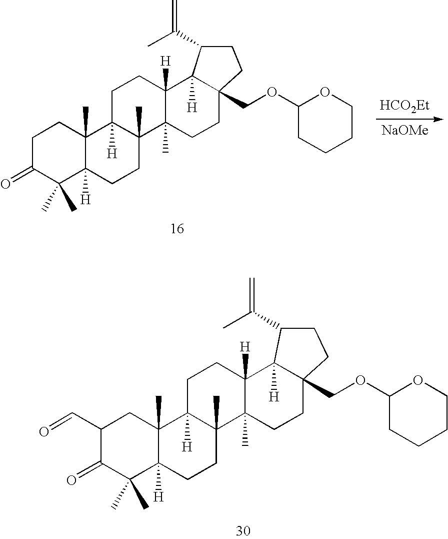

To a solution of 16 (1.2 g, 2.29 mmol) in anhydrous benzene (25 mL), while stirring at rt under N₂, was added NaOMe (0.80 g, 14.81 mmol). The resulting light orange suspension was stirred for 2 h, after which time it was quenched with 0.2 N HCl (50 mL). The organic layer was separated, washed with saturated NaHCO₃ (50 mL), dried over Na₂SO₄ and concentrated under vacuum. The crude product was purified by silica gel column chromatography eluting with a gradient of 0-20% EtOAc/heanex to afford 1.1 g (87% yield) of 30 as mixture of diastereomers (m.p. 150-163° C.). ¹H NMR (400 MHz, CDCl₃) δ 0.8-2.1 (m), 0.82 (s), 0.99 (s), 1.06 (s), 1.08 (s), 1.096 (s), 1.098 (s), 1.18 (s), 1.69 (s), 2.32 (d, J=14.4 Hz), 2.46 (m), 3.01 (d, J=9.6 Hz), 3.38 (d, J=9.2 Hz), 3.53 (m), 3.85 (m), 3.94 (d, J=9.6 Hz), 4.59 (m), 4.68 (s), 8.58 (d, J=2.8 Hz).

### Example 25

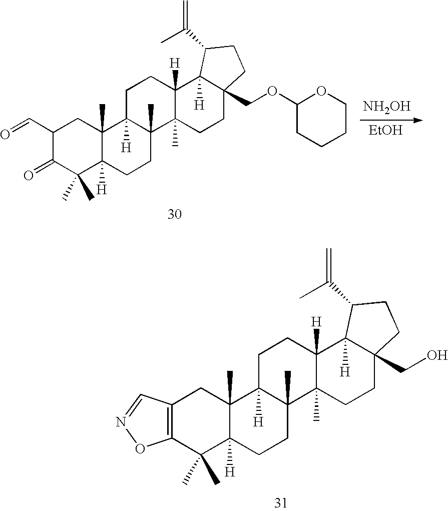

To a solution of 30 (41 mg, 0.074 mmol) in EtOH (10 mL), while stirring at rt under N₂ was added a solution of NH₂OH.HCl (52 mg, 0.74 mmol) in H₂ O (1 mL). The resulted clear solution was heated at reflux for 1 h, which was then cooled down to rt and concentrated under reduced pressure. The residue was dissolved in EtOAc (10 mL) and washed with saturated NaHCO₃ (10 mL). After being dried over Na₂SO₄ and concentrated under reduced pressure to an oil, which was purified by silica gel column chromatography eluting with a gradient of 0-50% EtOAc/hexane to provide 34 mg (99% yield) of 31 (m.p. 130-137° C.). ¹H NMR (400 MHz, CDCl₃) δ 0.8-2.1 (m, 21 H), 0.81 (s, 3H), 1.01 (s, 3H), 1.08 (s, 3H), 1.19 (s, 3H), 1.29 (s, 3H), 1.69 (d, J=0.8 Hz, 3H), 2.41 (m, 1H), 2.46 (d, J=14.8 Hz, 1H), 3.36 (d, J=10.8 Hz, 1H), 3.81 (d, J=11.2 Hz, 1H), 4.60 (m, 1H), 4.70 (d, J=2.0 Hz, 1H), 7.96 (s, 1H); ¹³C NMR (500 MHz, CDCl₃) γ 14.1, 14.7, 15.6, 16.0, 18.7, 19.0, 21.2, 21.3, 22.6, 25.1, 27.1, 28.6, 29.0, 29.7, 31.5, 33.1, 33.9, 34.8, 35.7, 37.3, 38.8, 40.9, 42.7, 47.75, 47.78, 48.6, 48.9, 53.4, 60.4, 108.8, 109.7, 150.2, 150.3, 173.0, Positive ESI-MS, m/e 466.7 (M−H)⁺.

### Example 26

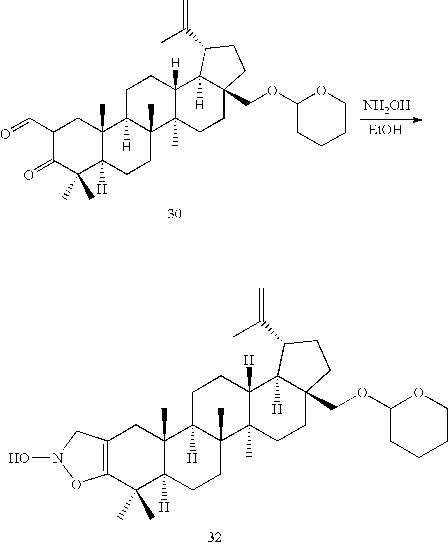

To a solution of 30 (123 mg, 0.22 mmol) in EtOH (30 mL), while stirring at rt under N₂, was added a solution of NH₂OH.HCl (156 mg, 2.22 mmol) in H₂O (3 mL) containing KOH (150 mg, 2.22 mmol). A noticeable amount of precipitates was formed and the resulting mixture heated at reflux for 1 h, whereupon it was cooled down to rt and concentrated under vacuum to a white solid, which was dissolved in EtOAc (30 mL) and washed with saturated NaHCO₃ (30 mL). The organic layer was dried over Na₂SO₄ and concentrated under reduced pressure. The residue was further purified by silica gel column chromatography eluting with a gradient of 5-50% EtOAc/hexane to provide 79 mg (63% yield) of 32 as a mixture of diastereomers (m.p. 187-190° C.). ¹H NMR (400 MHz, CDCl₃) δ 0.8-2.2 (m), 0.83 (s), 1.00 (s), 1.05 (s), 1.07 (s), 1.11 (s), 1.17 (s), 1.68 (s), 2.44 (m), 2.95 (d, J=18.8 Hz), 3.00 (d, J=4.4 Hz), 3.37 (d, J=9.2 Hz), 3.50 (m), 3.83 (m), 3.93 (d, J=9.2 Hz), 4.55 (t, J=2.8 Hz), 4.58 (m), 4.68 (s).

### Example 27

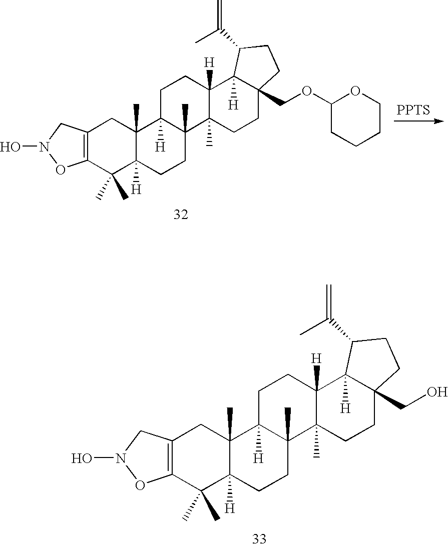

To a solution of 32 (47 mg, 0.085 mmol) in EtOH (4 mL), while stirring at rt, were added PPTS (22 mg, 0.085 mmol) and PTSA.H₂O (16 mg, 0.085 mmol). After 16 h, the reaction mixture was diluted with EtOAC (10 mL). The organic layer was separated, washed with saturated NaHCO₃ (10 mL), dried over Na₂SO₄ and concentrated under reduced pressure. The residue obtained was purified by silica gel column chromatography with gradient elution (20-50% EtOAc/hexane) to afford 32 mg (78% yield) of 33 as mixture of diastereomers (m.p. 227-231° C.). ¹H NMR (500 MHz, CDCl₃) δ 0.7-2.2 (m, 25 H), 0.83 (s, 3H), 1.01 (s, 3H), 1.06 (s, 3H), 1.11 (s, 3H), 1.17 (s, 3H), 1.69 (s, 3H), 2.40 (m, 1H), 2.96 (d, J=19 Hz, 1H), 3.36 (d, J=10.5 Hz, 1H), 3.80 (d, J=11 Hz, 1H), 3.59 (s, 1H), 4.69 (s, 1H).

### Example 28

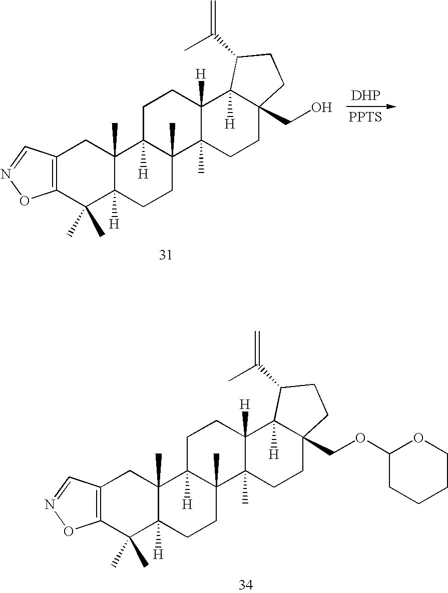

To a suspension of 31 (0.8 g, 1.72 mmol) in anhydrous CH₂Cl₂ (25 mL), while stirring at rt under N₂, was added dropwise 3,4-dihydro-2H-pyran (DHP, 0.17 mL, 1.86 mmol) followed by PPTS (51 mg, 0.20 mmol). After 16 h, the reaction was quenched with saturated NaHCO₃ (50 mL). The organic layer was separated, washed with brine (50 mL), dried over Na₂SO₄ and concentrated under reduced pressure. The crude residue was purified by silica gel column chromatography with gradient elution (5-20% EtOAc/hexane) to afford 858 mg (91% yield) of 34 as a mixture of diastereomers (m.p. 99-105° C.). ¹H NMR (400 MHz, CDCl₃) δ 0.8-2.1 (m), 0.80 (s), 1.00 (s), 1.07 (s), 1.08 (s), 1.195 (s), 1.197 (s), 1.29 (s), 1.69 (s), 2.46 (m), 3.01 (d, J=9.2 Hz), 3.39 (d, J=9.6 Hz), 3.53 (m), 3.85 (m), 3.94 (d, J=10 Hz), 4.55 (t, J=3.6 Hz), 4.59 (m), 4.69 (s), 4.95 (m), 7.96 (s).

### Example 29

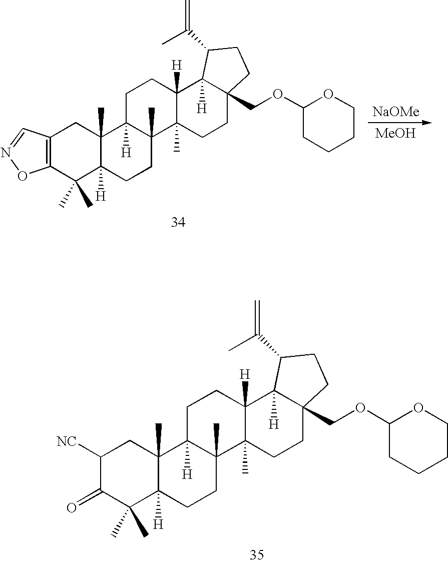

To a solution of 34 (227 mg, 0.41 mmol) in anhydrous ether (14 mL) and anhydrous MeOH (6 mL), while stirring at 0° C. under N₂, was added portionwise powder NaOMe (0.80 g, 14.81 mmol). The suspension was then warmed to rt and stirred for 1 h, whereupon it was diluted with EtOAc (15 mL). The organic layer was separate, washed with 0.2 N HCl (2×15 mL) and saturated NaHCO₃ (20 mL), dried over Na₂SO₄ and concentrated under vacuum. The crude product was purified by silica gel column chromatography (10% EtOAc/hexane) to afford 206 mg (91% yield) of 35 as a mixture of diastereomers (m.p. 186-197° C.). ¹H NMR (400 MHz, CDCl₃) δ 0.8-2.2 (m), 0.86 (s), 0.97 (s), 1.04 (s), 1.05 (s), 1.06 (s), 1.14 (s), 1.43 (s), 1.68 (s), 2.43 (m), 3.00 (d, J=9.6 Hz), 3.37 (d, J=3.35 Hz), 3.51 (m), 3.86 (m), 4.55 (t, J=3.2 Hz), 4.58 (m), 4.68 (s), 5.73 (d, J=1.6 Hz).

### Example 30

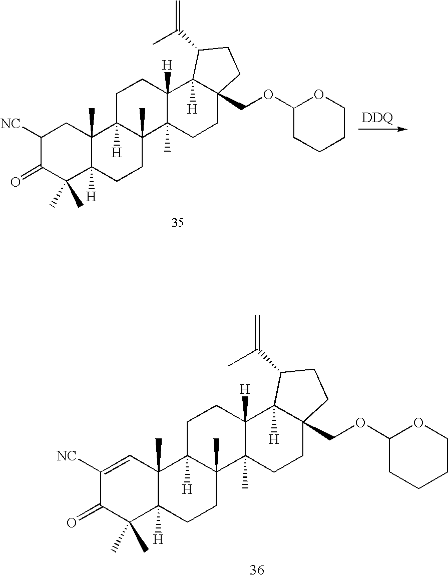

To a solution of 35(100 mg, 0.18 mmol) in benzene (8 mL), while stirring at rt, was added DDQ (60 mg, 0.26 mmol). The resulting dark orange solution was then heated at reflux for 1 h, whereupon it was cooled down to rt. The precipitated were filtered off and the filtrate was concentrated under reduced pressure to a crude residue which was then purified by silica gel column chromatography eluting with a gradient of 5-15% EtOAc/hexane to afford 71 mg of pure 36 (71% yield) as mixture of diastereomers (m.p. 180-193° C.). ¹H NMR (400 MHz, CDCl₃) δ 0.8-2.1 (m), 0.98 (s), 1.11 (s), 1.12 (s), 1.19 (s), 1.69 (s, 3H), 2.46 (m), 3.01 (d, J=9.2 Hz), 3.35 (d, J=8.8 Hz), 3.53 (d, J=9.2 Hz), 3.86 (m), 4.55 (t, J=3.2 Hz), 4.59 (m), 4.69 (s), 7.80 (s).

### Example 31

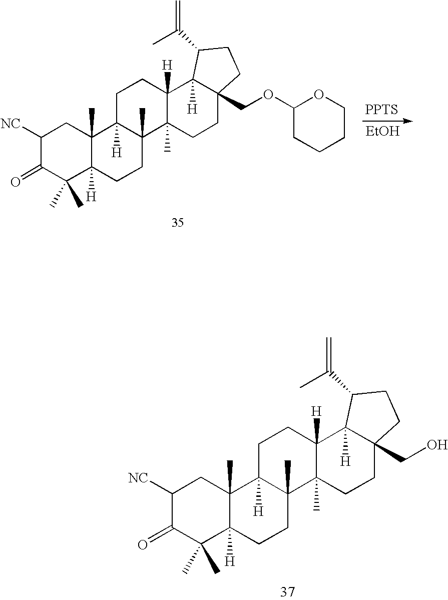

To a solution of 35(58 mg, 0. 11 mmol) in EtOH (4 mL), while stirring at rt, were added PPTS (26 mg, 0.11 mmol) and PTSA.H₂O (20 mg, 0.11 mmol). Afterr 16 h, the reaction mixture was diluted with EtOAc (10 mL). The organic layer was separated, washed with sat. NaHCO₃ (10 mL), dried over Na₂SO₄and concentrated under reduced pressure. The residue was purified by silica gel column chromatography with gradient elution (10-50% EtOAc/hexane) to afford 40 mg (81% yield) of 37 as mixture of diastereomers (m.p. 190-194° C.). ¹H NMR (500 MHz, CDCl₃) δ 0.8-2.2 (m), 0.80 (s), 0.87 (s), 0.98 (s), 1.05 (s), 1.15 (s), 1.24 (s), 1.35 (s), 1.36(s), 1.68 (s), 1.82 (m), 1.88 (m), 2.40 (m), 2.80 (d, J=11.2 Hz), 3.35 (d, J=11 Hz), 3.79 (t, J=9 Hz), 3.87 (m), 4.44 (s), 4.59 (s), 4.69 (s), 5.30 (s), 5.78 (s).

### Example 32

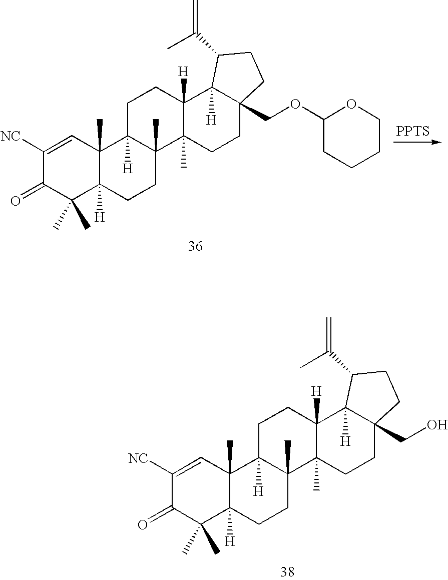

To a solution of 36 (40 mg, 0.073 mmol) in EtOH (4 mL), while stirring at rt, were added PPTS (18 mg, 0.073 mmol) and PTSA.H₂O (14 mg, 0.073 mmol). After 16 h, the reaction mixture was diluted with EtOAc (10 mL). The organic layer was separated, washed with saturated NaHCO₃ (10 mL), dried over Na₂SO₄ and concentrated under reduced pressure. The residual was purified by silica gel column chromatography with gradient elution (10-50% EtOAc/hexane) to afford 25 mg (75% yield) of 38 as a white solid (m.p. 170-175° C.). ¹H NMR (500 MHz, CDCl₃) δ 0.8-2.1 (m, 20H), 0.99 (s, 3H), 1.11 (s, 6H), 1.13 (s, 3H), 1.19 (s, 3H), 1.69 (s, 3H), 2.41 (ddd, J=5.5, 11, 17 Hz, 1H), 3.37 (dd, J=4, 11 Hz, 1H), 3.78 (1H, J=4, 11 Hz, 1H), 4.61 (s, 1H), 4.70 (s, 1H), 7.79 (s, 1H).

### Example 33

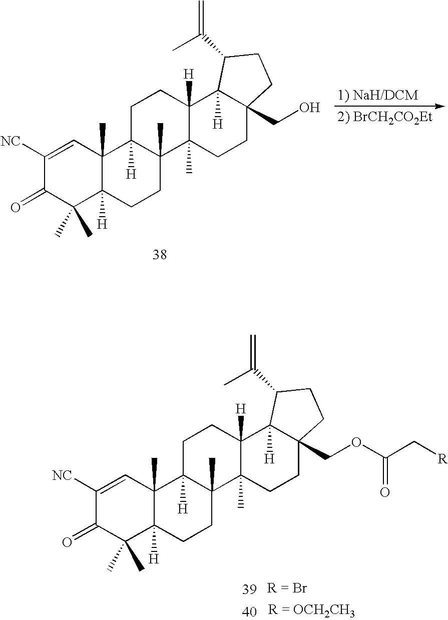

To a suspension of NaH (86 mg of 60% dispersion in mineral oil, 2.15 mmol) in anhydrous CH₂Cl₂ (2 mL), while stirring at rt under N₂, was added dropwise a solution of 38 (100 mg, 0.21 mmol) in anhydrous CH₂Cl₂ (1 mL). After 0.5 h, a solution of ethyl bromoacetate (75 mg, 0.43 mmol) in anhydrous CH₂Cl₂ (1 mL) was added dropwise. The resulting mixture was stirred at rt for 5 days, whereupon it was cooled to 0° C. and quenched with dropwise addition of H₂O (1 mL). The heterogeneous mixture was partitioned between EtOAc (10 mL) and brine (10 mL). The organic layer was separated, dried (Na₂SO₄) and concentrated under reduced pressure to an oily residue, which was purified by SiO₂ column chromatography with gradient elution gradient (0-20% EtOAc/hexane) to give 40 mg (36% yield) of 39 (m.p. 138-142° C.). ¹H NMR (500 MHz, CDCl₃) δ 0.9-2.1 (m, 21 H), 0.99 (s, 3H), 1.11 (s, 3H), 1.31 (s, 6H), 1.19 (s, 3H), 1.69 (s, 3H), 2.43 (m, 1H), 3.86 (s, 2H), 3.94 (d, J=11 Hz, 1H), 4.40 (d, J=11 Hz, 1H), 4.63 (m, 1H), 4.71 (m, 1H), 7.79 (s, 1H). ¹³C NMR (500 MHz, CDCl₃) δ 14.6, 16.5, 18.3, 18.8, 19.0, 21.1, 21.3, 24.9, 25.8, 26.9, 27.7, 29.41, 29.47, 33.2, 34.3, 37.7, 40.7, 42.0, 43.0, 43.7, 44.9, 46.5, 47.5, 48.5, 52.5, 64.5, 110.3, 114.0, 114.9, 149.4, 167.6, 170.5, 198.2.

### Example 34

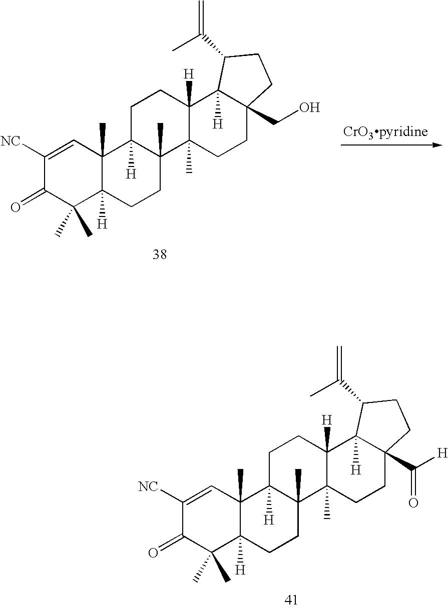

To a solution of pyridine (0.18 mL, 2.30 mmol) in CH₂Cl₂ (4 mL), while stirring at rt under N₂, was added CrO₃ (115 mg, 1.15 mmol). The resulting dark brown suspension was stirred for 1 h at rt, whereupon it was cooled to 0° C. A solution of 38 (89 mg, 0.192 mmol) in CH₂Cl₂ (2 mL) was added dropwise and stirring was continued at 0° C for an additional hour. The solids were filtered off and the filtrate was concentrated under reduced pressure. The residue obtained was purified by silica gel column chromatography (0-15% EtOAc/hexane) to afford 30 mg (34% yield) of 41 (m.p. 161-167° C.). ¹H NMR (500 MHz, CDCl₃) δ 0.8-2.2 (m, 20H), 0.98 (s, 3H), 1.01 (s, 3H), 1.11 (s, 3H), 1.12 (s, 3H), 1.18 (s, 3H), 1.70 (s, 3H), 2.89(m, 1), 4.66 (m, 1H), 4.77 (m, 1H), 7.79 (s, 1H), 9.64 (d, J=1.5 Hz, 1H).

### Example 35

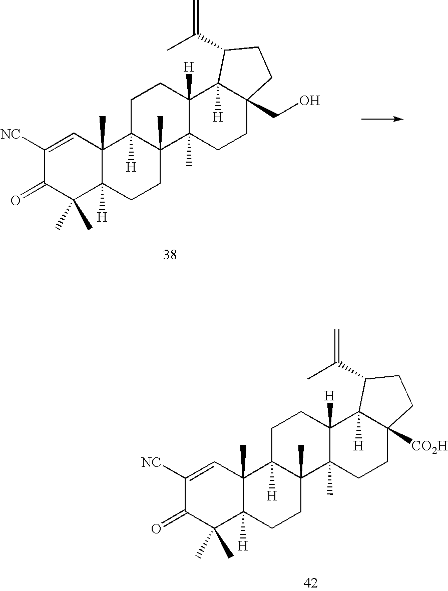

To a suspension of 38 (102 mg, 0.22 mmol) in acetone (3 mL), while stirring at 0° C. under N₂, was added dropwise Jones' reagent (0.24 mL, 1.96 M, 0.46 mmol). After 4 h, the red-orange reaction mixture was treated with sodium metabisulfite in three portions until the color was brown. The heterogeneous mixture was partitioned between H₂O (10 mL) and EtOAc (10 mL). The organic layer was separated, dried (Na₂SO₄) and concentrated under reduced pressure to a clear oil which was purified by SiO₂ silica gel column chromatography (0-50% EtOAc/hexane) to afford 60 mg (57% yield) of 42. ¹H NMR (500 MHz, CDCl₃) δ 0.9-2.4 (m, 21H), 0.99 (s, 3H), 1.03 (s, 3H), 1.115 and 1.119 (2s, 3H each), 1.18 (s, 3H), 1.70 (s, 3H), 3.02 (m, 1H), 4.64 (s, 1H), 4.76 (s, 1H), 7.81 (s, 1H).

### Example 36

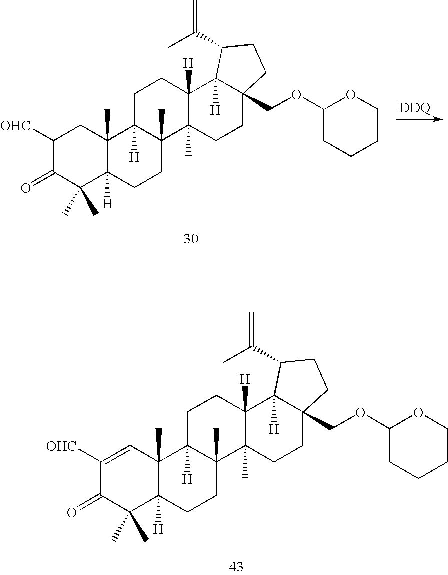

To a solution of 30(100 mg, 0.18 mmol) in benzene (8 mL), while stirring at rt, was added DDQ (60 mg, 0.26 mmol). The resulting dark orange solution was heated at reflux for 1 h, whereupon it was cooled down to rt and the solids were filtered off. The filtrate was concentrated under reduced pressure. The crude residue was purified by silica gel column chromatography eluting with a gradient of 5-15% EtOAc/hexane to afford 71 mg of the aldehyde 43 which was assigned as a 1: I mixture of starting material 30 and product 43 both as mixture of diastereomers.

### Example 37

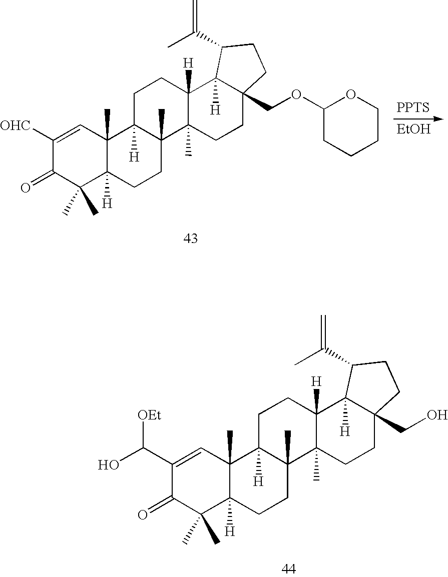

To a solution of 43 (31 mg, 0.06 mmol) in EtOH (4 mL), while stirring at rt, were added PPTS (14 mg, 0.06 mmol) and PTSA.H₂O (11 mg, 0.06 mmol). After 16 h, the reaction mixture was diluted with EtOAc (10 mL). The organic layer was separated, washed with saturated NaHCO₃ (10 mL), dried (Na₂SO₄) and concentrated under reduced pressure. The residual was purified by silica gel column chromatography with gradient elution (10-50% EtOAc/hexne to afford 18 mg (59% yield) of a compound structure of which was assigned as a diastereomeric mixture of two ethyl hemiacetals of 44 as shown by ¹H NMR analysis.

### Example 38

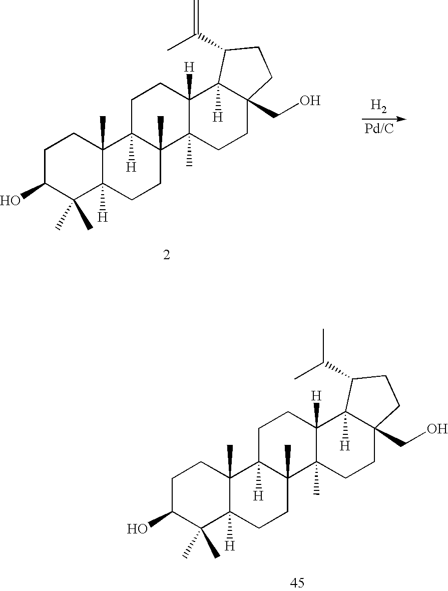

A suspension of betulin (2) (5.0 g, 11.29 mmol) and Pd/C (10% wt on activated carbon) (1.0 g) in MeOH (75 mL) and CH₂Cl₂ (100 mL) was hydrogenated at 35 psi for 16 h. The suspension was filtered through Celite and the solids were washed with additional MeOH (300 mL), acetone (300 mL) and THF (500 mL). The combined filtrates were concentrated under reduced pressure to afford 5.0 g (quantitative) of 45 (m.p. 277-279° C.) which was used directly in the next step. ¹H NMR (500 MHz, DMSO-d₆) δ 0.8-2.1 (m), 0.66 (s, 3H), 0.72 (d, J=6.5 Hz, 3H), 0.77 (s, 3H), 0.81 (d, J=6.5 Hz, 3H), 0.87 (s, 3H), 0.90 (s, 3H), 0.98 (s, 3H), 2.99 (m, 21H), 3.50 (m, 1H), 4.14 (m, 1H), 4.26 (d, J=5 Hz, 1H).

### Example 39

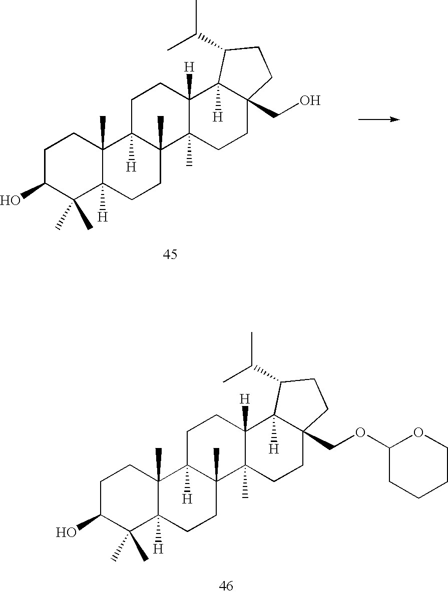

To a solution of 45 (4.0 g, 8.99 mmol) in anhydrous THF (120 mL) and anhydrous CH₂Cl₂ (200 mL), while stirring at rt under N₂, was added dropwise 3,4-dihydro-2H-pyran (0.92 g, 10.96 mmol), followed by addition of PPTS (0.5 g, 1.99 mmol). After 4 days, the reaction was quenched with saturated NaHCO₃ (200 mL). The organic layer was separated, washed with brine (100 mL), dried (Na₂SO₄) and concentrated under reduced pressure to a crude solid, which was further purified by SiO₂ column chromatography with gradient elution (0-50% EtOAc/hexane) to afford 2.0 g (52% yield) of compound 46 (m.p. 189-190° C.) as a mixture of diastereomers and 2.9 g of a solid which was assigned as a mixture of 46 and a bis-THP protected material by TLC analysis. Compound 46: ¹H NMR (400 MHz, CDCl₃) δ 0.6-2.1 (m), 0.76 (s), 0.83 (s), 0.95 (s), 0.97 (s), 1.02 (s), 1.04 (s), 2.95 (d, J=9.5 Hz), 3.18 (dd, J=4.5, 11.5 Hz), 3.35 (d, J=9 Hz), 3.47 (d, J=9 Hz), 3.52 (m), 3.83 (m), 3.90 (d, J=9.5 Hz), 4.52 (d, J=9 Hz), 4.57 (d, J=9 Hz).

### Example 40

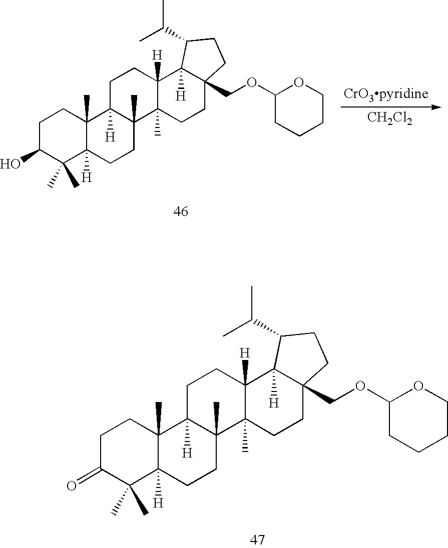

To a solution of pyridine (3.67 mL, 45.36 mmol) in CH₂Cl₂ (60 mL), while stirring at rt under N₂, was added CrO₃ (2.27 g, 22.69 mmol). The resulting dark brown suspension was stirred for 1 h at rt, whereupon it was cooled to 0° C. and a solution of 46 (2.0 g, 3.78 mmol) in CH₂Cl₂ (10 mL) was added dropwise. The suspension was stirred at 0° C for an additional hour. The solids were filtered off and the filtrate was concentrated under reduced pressure. The resulting residue was purified by silica gel column chromatography eluting with CH₂Cl₂/hexane (1:1) followed by 20-50% EtOAc/hexane to afford 1.3 g (65% yield) of 47 as a mixture of diastereomers. This material was pure by TLC analysis and identical to an authentic sample obtained from a previous experiment. No further analytical data was obtained for this product.

### Example 41

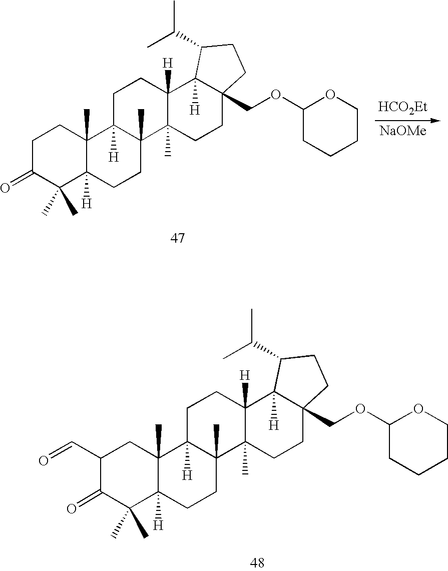

To a solution of 47 (220 mg, 0.417 mmol) and ethyl formate (0.15 mL, 1.84 mmol) in anhydrous benzene (4 mL), while stirring at rt under N₂, was added NaOMe (0.13 g, 2.40 mmol). The resulting yellow suspension was stirred for 2 h, diluted with EtOAc (5 mL) and acidified with 0.2 N HCl (10 mL). The organic layer was separated, washed with saturated NaHCO₃ (10 mL), dried (Na₂SO₄) and concentrated under reduced pressure to afford 221 mg (96% yield) of 48 as a mixture of diastereomers. This material was taken to the next step without further purification.

### Example 42

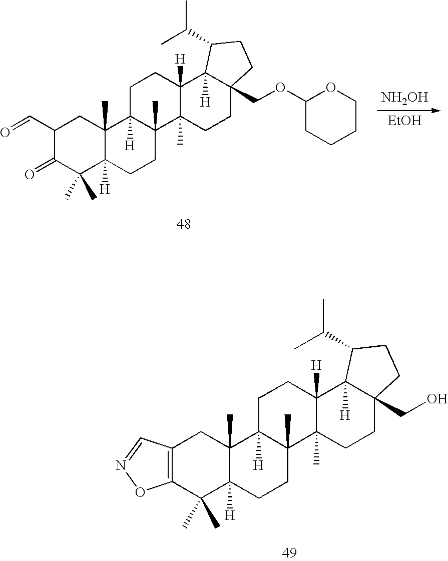

To a solution of 48 (221 mg, 0.39mmol) in EtOH (50 mL), while stirring at rt under N₂, was added a solution of NH₂OH.HCl (280 mg, 4.03 mmol) in H₂O (5 mL). The solution was then heated at 90° C. for 2 h, whereupon it was cooled down to rt and concentrated under reduced pressure. The residue was partitioned between EtOAc (10 mL) and water (2 mL). The organic layer was separated, washed with sat. NaHCO₃ (10 mL), dried (Na₂SO₄) and concentrated under reduced pressure to an oil, which was purified by silica gel column chromatography eluting with a gradient of 20-40% EtOAc/hexane to provide 179 mg (92% yield from 47) of 49 (m.p. 139-145° C.). ¹H NMR (400 MHz, CDCl₃) δ 0.78-2.0 (m, 24 H), 0.78 (d, J=7 Hz, 3H), 0.82 (s, 3H), 0.86 (d, J=7 Hz, 3H), 0.99 (s, 3H), 1.08 (s, 3H), 1.20 (s, 3H), 1.30 (s, 3H), 2.48 (d, J=15 Hz, 1H), 3.33 (d, J=11 Hz, 1H), 3.79 (d, J=10.5 Hz, 1H), 7.97 (s, 1H); ¹³C NMR (500 MHz, CDCl₃) δ 14.5, 14.8, 15.7, 16.0, 18.7, 21.2, 21.3, 21.7, 22.9, 26.8, 27.0, 28.6, 29.2, 29.5, 33.2, 34.0, 34.8, 35.7, 36.9, 38.8, 40.9, 42.9, 44.5, 47.9, 48.0, 48.6, 53.4, 60.5, 108.8, 150.2, 173.0.

### Example 43

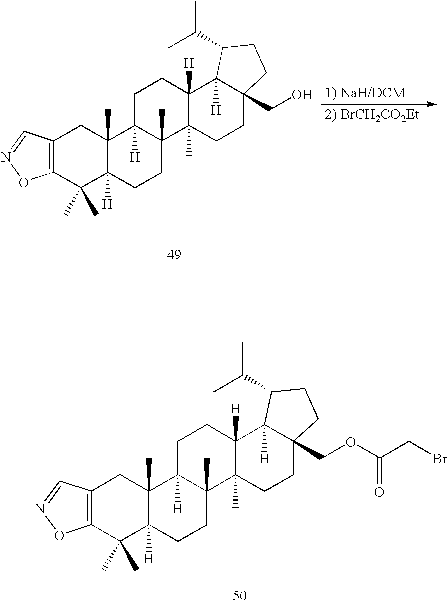

To a suspension of NaH (81 mg of 60% dispersion in mineral oil, 2.03 mmol) in anhydrous CH₂Cl₂ (2 mL), while stirring at rt under N₂, was added dropwise a solution of 49 (95 mg, 0.20 mmol) in anhydrous CH₂Cl₂ (1 mL). After 0.5 h, a solution of ethyl bromoacetate (71 mg, 0.42 mmol) in anhydrous CH₂Cl₂ (1 mL) was added dropwise. The resulting mixture was stirred at rt for 5 days, whereupon it was cooled to 0° C. and quenched with careful addition of H₂O (1 mL). The heterogeneous mixture was partitioned between EtOAc (10 mL) and water (10 mL). The aqueous layer was extracted with EtOAc (10 mL) and the combined organic layers were dried (Na₂SO₄) and concentrated under reduced pressure to an oily residue, which was purified by SiO₂ column chromatography eluting with a gradient of 10-50% EtOAc/hexane to give 81 mg (36% yield) of 50 (m.p. 188-197° C.). ¹H NMR (400 MHz, CDCl₃) δ 0.76-2.1 (m, 22 H), 0.78 (d, J=6.8 Hz, 3H), 0.82 (s, 3H), 0.86 (d, J=6.8 Hz, 3H), 0.98 (s, 3H), 1.09 (s, 3H), 1.20 (s, 3H), 1.30 (s, 3H), 1.95 (d, J=15.2 Hz, 1H), 2.49 (d, J=15.2 Hz, 1H), 3.86 (s, 2H), 3.94 (d, J=11 Hz, 1H), 4.40 (d, J=11 Hz, 1H), 7.98 (s, 1H).

### Example 44

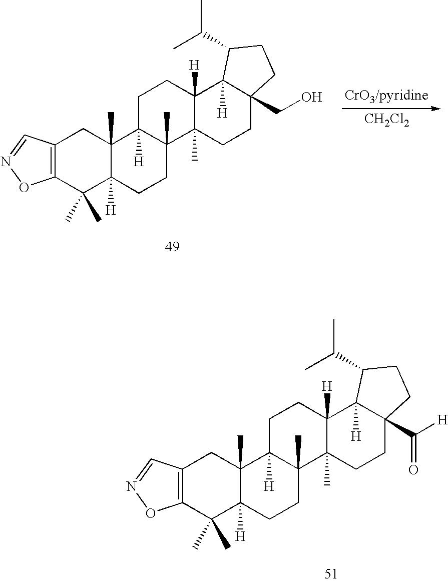

To a solution of pyridine (80 μL, 1.03 mmol) in CH₂Cl₂ (2 mL), while stirring at rt under N₂, was added CrO₃ (51 mg, 1.15 mmol). The resulting dark brown suspension was stirred for 0.5 h and cooled to 0° C. A solution of 49 (40 mg, 0.086 mmol) in CH₂Cl₂ (1 mL) was added dropwise. The suspension was stirred at 0° C. for an additional hour. The solids were filtered off and the filtrate was concentrated under reduced pressure. The resulting residue was purified by silica gel column chromatography (5-40% EtOAc/hexane) to afford 24 mg (60% yield) of 51 (m.p. 229-238° C.). ¹H NMR (500 MHz, CDCl₃) δ 0.78-2.2 (m, 22H), 0.79 (d, J=7 Hz, 3H), 0.81 (s, 3H), 0.89 (d, J=7 Hz, 3H), 0.96 (s, 3H), 0.97 (s, 3H), 1.19 (s, 3H), 1.29 (s, 3H), 1.95 (d, J=15 Hz, 1H), 2.50 (d, J=15 Hz, 1H), 7.97 (s, 1H), 9.64 (s, 1H).

### Example 45

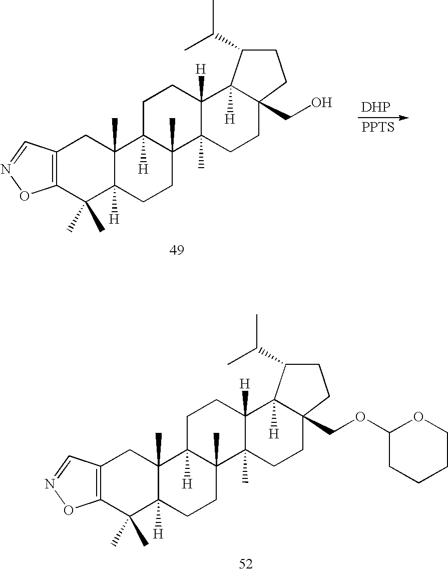

To a suspension of 49 (1.1 g, 2.35 mmol) in anhydrous CH₂Cl₂ (30 mL), while stirring at rt under N₂, was added dropwise 3,4-dihydro-2H-pyran (DHP, 0.24 g, 2.82 mmol), followed by addition of PPTS (120 mg, 0.47 mmol). After 16 h, a mixture of sat. NaHCO₃ (40 mL) and brine (10 mL) was added. The aqueous layer was extracted with EtOAc (50 mL) and the combined organic layers were dried (Na₂SO₄) and concentrated under reduced pressure. The crude residue was purified by silica gel column chromatography with gradient elution (0-10% EtOAc/hexane) to afford 1.0 g (78% yield) of pure 52 as a mixture of diastereomers (m.p. 106-110° C.). ¹H NMR (500 MHz, CDCl₃) δ 0.7-2.1(m), 0.78 (d, J=6.5 Hz), 0.81 (s), 0.85 (d, J=7 Hz), 0.98 (s), 1.08 (s), 1.09 (s), 1.30(s), 1.95 (d, J=15 Hz), 2.48 (d, J=15 Hz), 2.97 (d, J=9.5 Hz), 3.37 (d, J=9 Hz), 3.50 (m), 3.85 (m), 3.92 (d, J=9.5 Hz ), 4.53 (t, J=3.5 Hz), 4.58 (t, J=3.5 Hz), 4.95 (m), 7.97 (s).

### Example 46

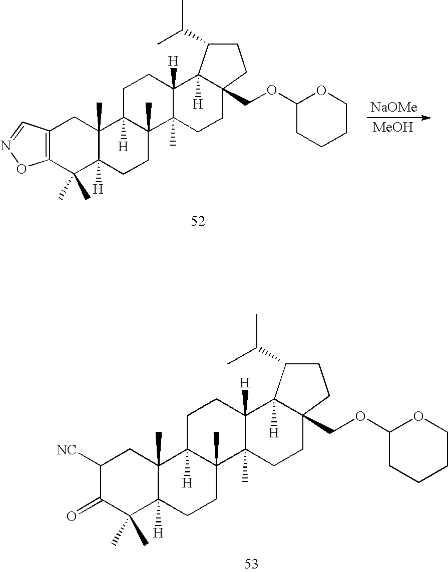

To a solution of 52 (1.0 g, 1.81 mmol) in anhydrous ether (65 mL) and anhydrous MeOH (25 mL), while stirring at 0° C. under N₂, was added portionwise powder NaOMe (3.52 g, 65.23 mmol). The suspension was warmed to rt and stirring was continued for 2 h. The reaction mixture was then quenched with H₂O (5 mL), diluted with EtOAc (100 mL) and acidified with 0.2 N HCl (100 mL). The organic layer was separated, washed with saturated NaHCO₃ (100 mL), dried (Na₂SO₄) and concentrated under reduced pressure. The crude product was purified by silica gel column chromatography eluting with a gradient of 0-50% EtOAc/hexane to afford 0.9 g (90% yield) of 53 as a mixture of diastereomers (m.p. 130-135° C.). ¹H NMR (400 MHz, CDCl₃) δ 0.74-2.1 (m), 0.77 (d, J=6.5 Hz), 0.84 (d, J=6.5 Hz), 0.87 (s), 0.88 (s), 0.95 (s), 1.04 (s), 1.055 (s), 1.058 (s), 1.06 (s), 1.15 (s), 1.83 (d, J=15 Hz), 2.15 (d, J=15 Hz), 2.96 (d, J=9 Hz), 3.35 (d, J=9 Hz), 3.48 (d, J=9.5 Hz), 3.52 (m), 3.85 (m), 4.53 (t, J=3.5 Hz), 4.58 (t, J=3 Hz), 5.685 (s), 5.688 (s).

### Example 47

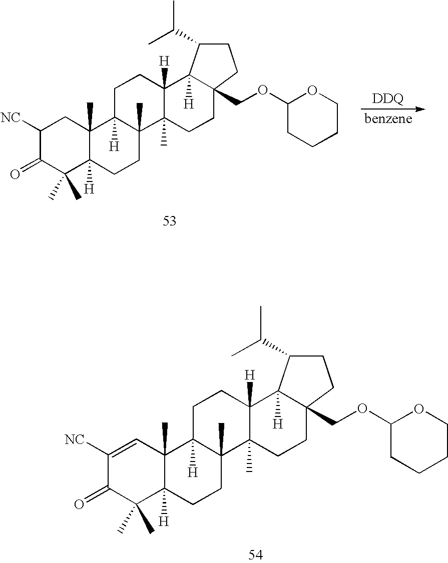

To a solution of 53 (0.9 g, 1.63 mmol) in benzene (60 mL), while stirring at rt, was added DDQ (0.5 g, 2.20 mmol). The resulting dark orange solution was heated at reflux for 1 h, whereupon it was cooled down to rt. The solids were filtered and washed with EtOAc (20 mL). The combined filtrates were concentrated under reduced pressure. The crude product was purified by SiO₂ column chromatography eluting with a gradient of 5-15% EtOAc/hexane to afford 426 mg of pure 54 (48% yield) as a mixture of diastereomers (m.p. 172-178° C.). ¹H NMR (400 MHz, CDCl₃) δ 0.76-2.1 (m), 0.78 (d, J=6.5 Hz), 0.86 (d, J=7 Hz), 0.96 (s), 1.11 (s), 1.12 (s), 1.13(s), 1.19 (s), 2.98 (d, J=9.5 Hz), 3.33 (d, J=9.5 Hz), 3.49 (d, J=10 Hz), 3.53 (m), 3.83 (m), 3.89 (d, J=9.5 Hz), 4.52 (t, J=3 Hz), 4.58 (t, J=3 Hz), 7.80 (t, J=2 Hz).

### Example 48

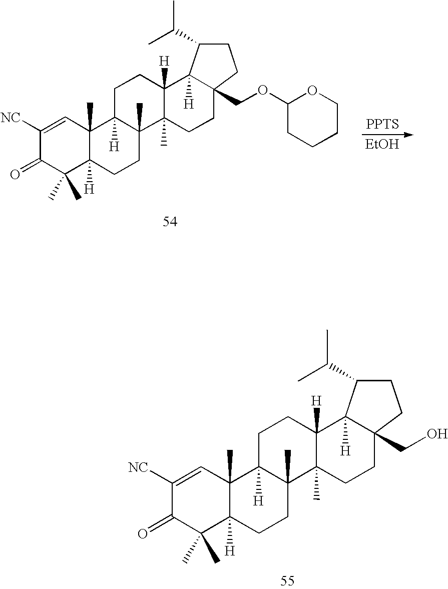

To a solution of 54 (400 mg, 0.72 mmol) in EtOH (40 mL), while stirring at rt, were added PPTS (183 mg, 0.72 mmol) and PTSA.H₂O (138 mg, 0.72 mmol). After 16 h, the reaction mixture was diluted with EtOAC (100 mL) and washed with saturated NaHCO₃ (2×100 mL). The organic layer was dried (Na₂SO₄) and concentrated under reduced pressure. The crude product was purified by SiO₂ column chromatography with gradient elution (0-50% EtOAc/hexane) to afford 286 mg (85% yield) of 55 as white solid (m.p. 168-171° C.). ¹H NMR (500 MHz, CDCl₃) δ 0.76-2.0 (m, 23H), 0.78 (d, J=7 Hz, 3H), 0.87 (d, J=6.5 Hz, 1H), 3H), 1.12 (s, 6H), 1.13 (s, 3H), 1.19 (s, 3H), 3.33 (d, J=11 Hz, 1H), 3.75 (d, J=10.5 Hz, 1H), 7.80 (s, 1H).

### Example 49

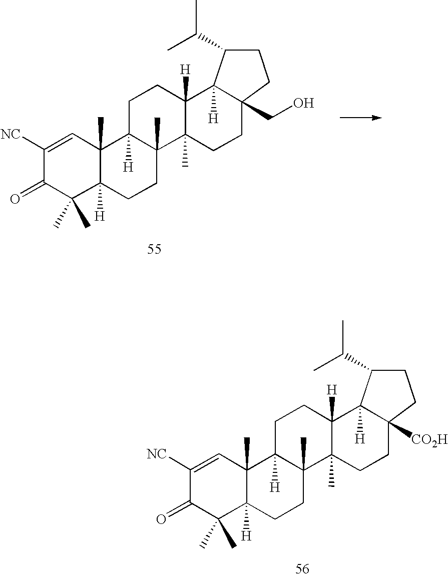

To a suspension of 55 (86 mg, 0.18 mmol) in acetone (3 mL), while stirring at 0° C. under N₂, was added dropwise Jones' reagent (0.20 mL, 1.96 M, 0.38 mmol). After 1 h, the red-orange reaction mixture was treated with sodium metabisulfite in three portions until the color was brown. The heterogeneous mixture was partitioned between H₂O (10 mL) and EtOAc (10 mL). The organic layer was separated, dried (Na₂SO₄) and concentrated under reduced pressure to a clear oil which was purified by SiO₂ silica gel column chromatography (0-50% EtOAc/hexane) to afford 50 mg (56% yield) of 56 (m.p. 216-218° C.). ¹H NMR (500 MHz, CDCl₃) δ 0.7-2.4 (m, 23H), 0.77 (d, J==7 Hz, 3H), 0.88 (d, J=7 Hz, 3H), 0.97 (s, 3H), 1.03 (s (s, 3H), 7.81 (s, 1H).

### Example 50

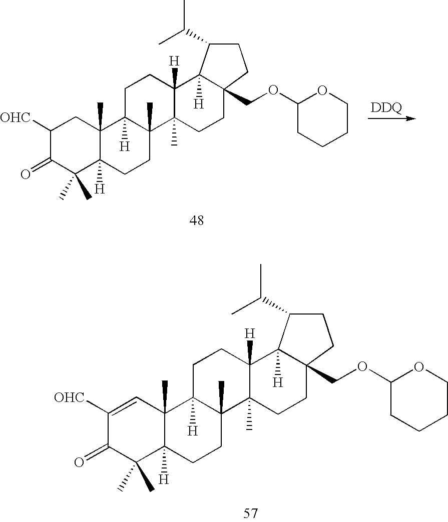

To a solution of 48 (1.0 g, 1.80 mmol) in benzene (60 mL), while stirring at rt, was added DDQ (60 mg, 0.26 mmol). The resulting dark orange solution was heated at reflux for 1 h and then cooled down to rt. The solids were filtered and washed with CH₂Cl₂ (20 mL). The combined filtrates were concentrated under reduced pressure to provide a crude residue which was partially purified by silica gel column chromatography (5-15% EtOAc/hexane to afford 107 mg of the aldehyde 57 as a mixture of diastereomers.

### Example 51

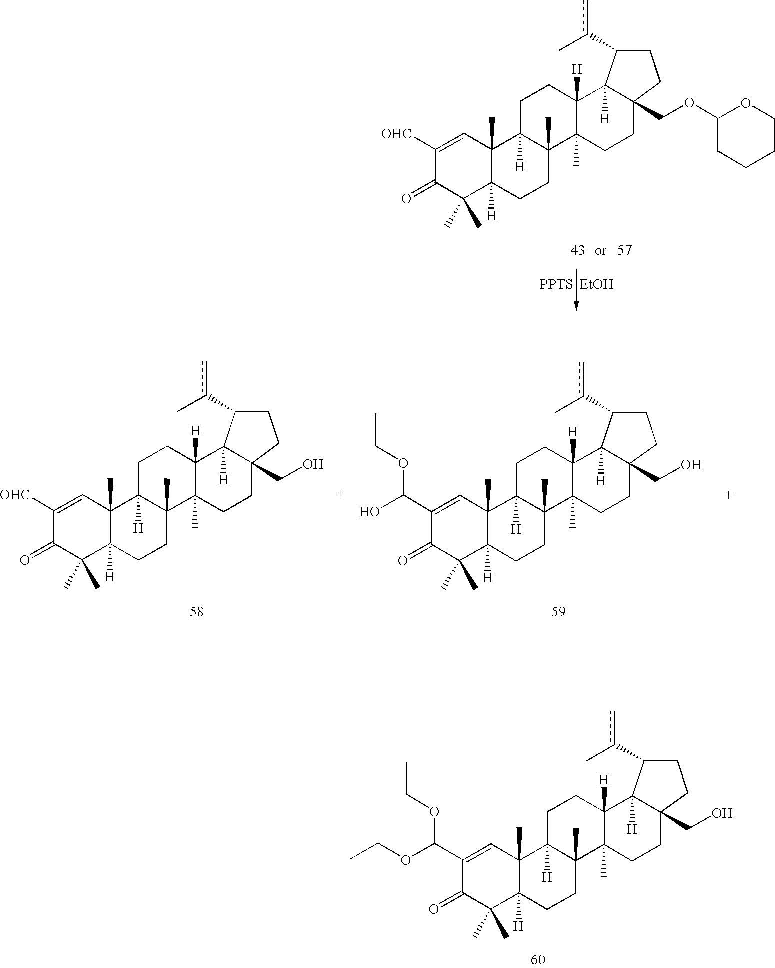

To solution of 43 (or 57) (163 mg, 0.30 mmol) in EtOH (2 mL), while stirring at rt, were added PPTS (74 mg, 0.30 mmol) and PTSA.H₂O (56 mg, 0.30 mmol) in succession. The reaction mixture was stirred for 48 h, partitioned between EtOAc (10 mL) and H₂O (10 mL). The organic layer was separated, washed with saturated NaHCO₃ (10 mL), dried (Na₂SO₄) and concentrated under reduced pressure. Purification by column chromatography yielded products 58, 59 and 60.

### Example 52

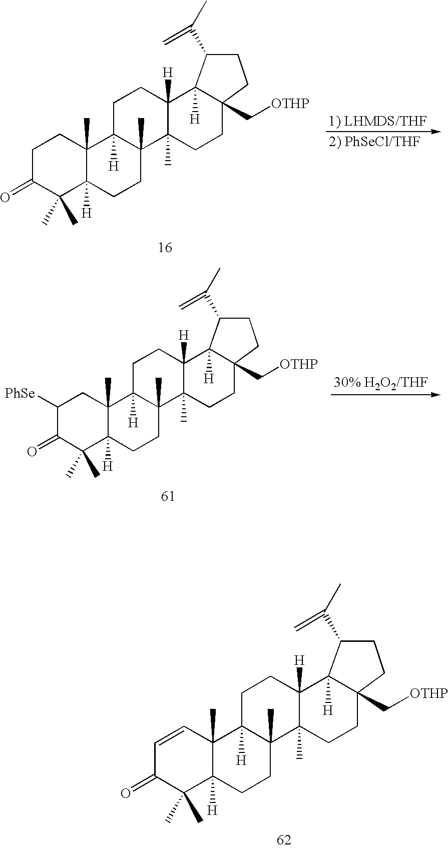

To solution of 16 (700 mg, 1.33 mmol) in THF (7 mL), while stirring at 0° C. under N₂, was added dropwise a 1 M solution of LHMDS in THF (2.66 mL, 2.66 mmol). After 1 h, a solution of PhSeCl (306 mg, 1.59 mmol) in THF (3 mL) was added dropwise. The temperature was gradually icreased to room temperature and, after stirring for an additional hpur, the reaction mixture was diluted with EtOAc (10 mL) and washed successively with 0.2 N HCl (10 mL) and saturated NaHCO₃ (10 mL). The organic layer was separated, dried (Na₂SO4) and concentrated under reduced pressure to a crude residue which was purified by silica gel column chromatography using gradient EtOAc/hexanes (0-20%) to give 788 mg (87% yield) of 61 as a white fluffy powder.

To a solution of 61 (576 mg, 0.85 mmol) in CH₂Cl₂ (3 mL), while stirring at 0° C., was added dropwise a 30% aqueous solution of H₂O₂ (0.4 mL, 3.53 mmol). After 1 h, a white precipitate had been formed and the reaction mixture was diluted with EtOAc (10 mL) and washed successively with saturated NaHCO₃ (10 mL) and saturated NaCl (10 mL) solutions. The organic layer was separated, dried (Na₂SO₄) and concentrated under reduced pressure. The crude product obtained was purified by silica gel column chromatography using gradient EtOAc/hexanes (0-15%) to afford 112 mg (25% yield) of 62 as a white fluffy powder. ¹H NMR (500 MHz, CDCl₃) δ 0.80-2.1 (m), 0.98 (s), 1.05 (s), 1.07 (s), 1.08 (s), 1.09 (s), 1.11 (s), 1.13 (s), 1.69 (s), 2.46 (m), 3.0 (d, J=10 Hz), 3.37 (d, J=8 Hz), 3.54 (m), 3.85 (m), 3.93 (d, J=9.5 Hz), 4.55 (t, J=3.5 Hz), 5.59 (m), 4.69 (s), 5.78 (d, J=10.5 Hz), 7.09 (dd, J=10.5 and 1.5 Hz).

### Example 53

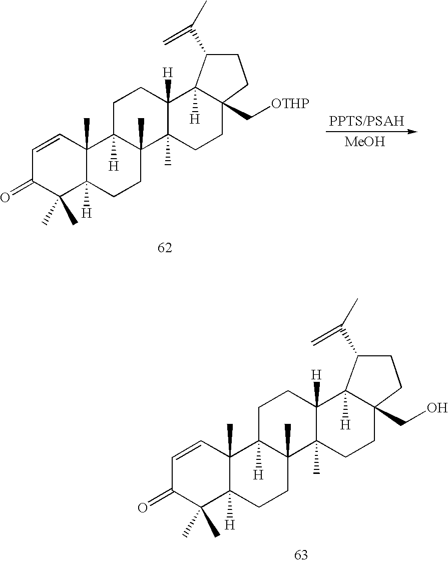

To a solution of 62 (47 mg, 0.09 mmol) in MeOH (2 mL) were added pyridinium p-toluenesulfonate (20mg, 0.08 mmol) and p-toluenesulfonic acid monohydrate (20 mg, 0.11 mmol) in succession. The reaction mixture was stirred at room temperature for 16 h, whereupon it was diluted with EtOAc (5 mL) and washed with saturated NaHCO₃ (5 mL) solution. The organic layer was separated, dried (Na₂SO₄) and concentrated under reduced pressure. Purification by SiO₂ column chromatography with gradient elution (0-50% EtOAc/hex) afforded 38 mg (97% yield) of 63 as white solid. ¹H NMR (500 MHz, CDCl₃) δ 0.8-2.1 (m), 0.99 (s), 1.06 (s), 1.08 (s), 1.11 (s), 1.13 (s), 1.68 (s), 2.37 (m), 3.36 (m), 3.81 (m), 4.59 (m), 4.70 (m), 5.79 (d,J=10.0 Hz), 7.08 (d,J=10.5 Hz).

### Example 54

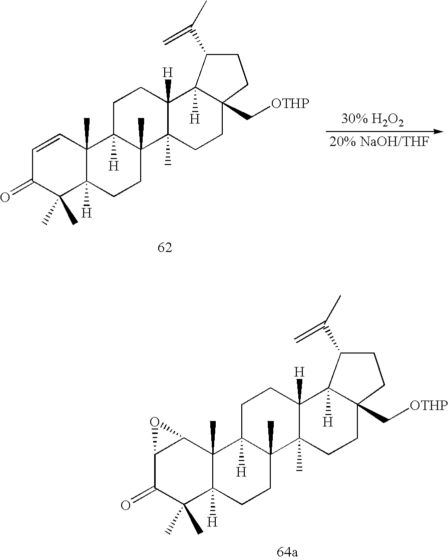

To a solution of 62 (100 mg, 0.20 mmol) in MeOH (2 mL), while stirring at 0° C., was added dropwise 30% aqueous solution of H₂O₂ (0.1 mL, 1.0 mmol) which was followed by the addition of 20% aqueous NaOH (0.2 mL, 1.0 mmol). After stirring at 0° C. for 0.5 h, the reaction mixture was warmed up to room temperature and stirred for additional 3 h, whereupon it was diluted with THF (1 mL). The reaction mixture was then stirred for 1 h and partitioned between EtOAc (10 mL) and H₂O (10 mL). The aqueous layer was extract with EtOAc (10 mL). The combined organic layers were dried (Na₂SO₄) and concentrated under reduced pressure to a white foam, which was purified by silica gel column chromatography (10-50% EtOAc/hexanes) to afford 88 mg (85% yield) of 64a as a mixture of diasteromers. ¹H NMR (500 MHz, CDCl₃) δ 0.8-2.1 (m), 0.87 (s), 0.97 (s), 0.98 (s), 1.03 (s), 1.06 (s), 1.07 (s), 1.08 (s), 1.69 (s), 2.45 (s), 3.00 (d, J=9.5 Hz), 3.34 (d, J=5 Hz), 3.37 (d, J=9.5 Hz), 3.52 (m), 3.54 (d, J=5 Hz), 3.85 (m), 3.94 (d, J=9.5 Hz), 4.55 (t, J=3.5 Hz), 4.59 (m), 4.68 (s).

### Example 55

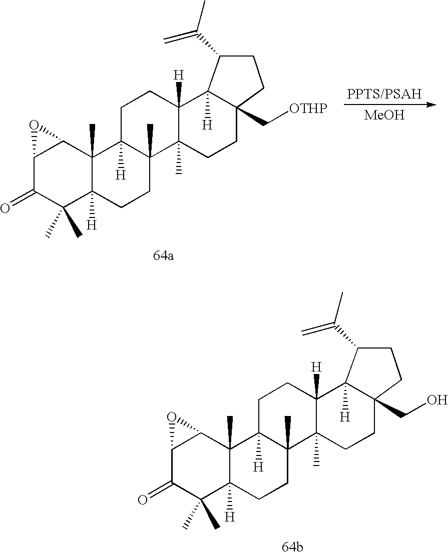

To a solution of 64a (103 mg, 0.19 mmol) in MeOH (3 mL) were added in succession pyridinium p-toluenesulfonate (60 mg, 0.24 mmol) and p-toluenesulfonic acid monohydrate (40 mg, 0.21 mmol). The reaction mixture was stirred at room temperature for 16 h, whereupon it was diluted with EtOAc (10 mL) and washed with saturated NaHCO₃ (10 mL) solution. The aqueous layer was extracted with EtOAc (10 mL) and the combined organic layers were dried (Na₂SO₄) and concentrated under reduced pressure. Purification by SiO₂ column chromatography (50% EtOAc/hexane and then 10% MeOH/CH₂Cl₂) afforded 90 mg (100% yield) of 64b as white solid. ¹H NMR (500 MHz, CDCl₃) δ 0.9-2.1 (m), 0.88 (s), 0.98 (s), 1.04 (s), 1.06 (s), 1.09 (s), 1.69 (s), 2.40 (m), 3.34 (d, J=4 Hz), 3.36 (m), 3.54 (d, J=5 Hz), 3.79 (m), 4.60 (m), 4.69 (m).

### Example 56

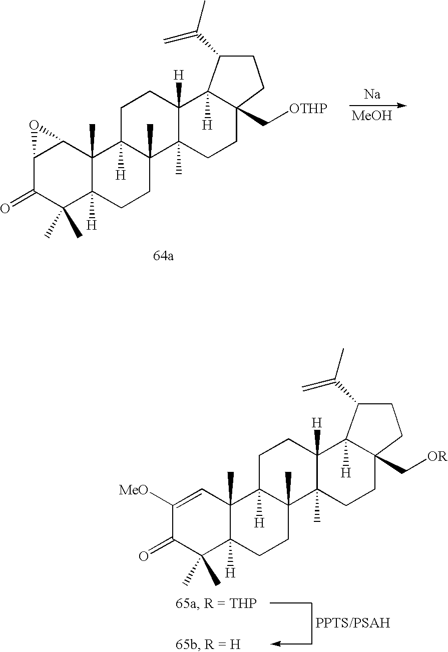

To a solution of 64a (40 mg, 0.07 mmol) in anhydrous MeOH (5 mL), while stirring at room temperature, was added Na (70 mg, 3.04 mmol) in pieces. After the metal was dissolved completely, the homogeneous mixture was heated at reflux for 48 h, whereupon it was cooled down to room temperature and MeOH removed under reduced pressure. The resulting residue was dissolved in EtOAc (10 mL) and washed successively with 10% HCl (10 ml) and saturated NaHCO₃ (10 mL) solution. The organic layer was separated, dried (Na₂SO₄) and concentrated under reduced pressure. The crude product obtained was purified by silica gel column chromatography to afford 34 mg (83% yield) of 65a. ¹H NMR (500 MHz, CDCl₃) δ 0.8-2.1 (m), 1.00 (s), 1.07 (s), 1.09 (s), 1.11 (s), 1.12 (s), 1.15 (s), 1.70 (s), 2.47 (m), 3.00 (d, J=9.5 Hz), 3.38 (d, J=8.5 Hz), 3.55 (s), 3.85 (m), 3.93 (d, J=8 Hz), 4.55 (t, J=3 Hz), 4.56 (m), 4.70 (s), 6.03 (d, J=2 Hz).

To a solution of 65a (24 mg, 0.04 mmol) in MeOH (2 mL), while stirring at room temperature, were added in succession pyridinium p-toluenesulfonate (12 mg, 0.05 mmol) and p-toluenesulfonic acid monohydrate (13 mg, 0.07 mmol). The reaction mixture was stirred for 16 h, whereupon it was diluted with EtOAc (10 mL) and washed with saturated NaHCO₃ (10 mL) solution. The organic layer was separated, dried (Na₂SO₄) and concentrated under reduced pressure. Purification by SiO₂ column chromatography with gradient elution (50% EtOAc/hexane) afforded 24 mg (100% yield) of 65b as white solid. ¹H NMR (500 MHz, CDCl₃) δ 0.8-2.1 (m), 1.01 (s), 1.07 (s), 1.10 (s), 1.12 (s), 1.15 (s), 1.70 (s), 2.41 (m), 3.36 (m), 3.55 (s), 3.80 (m), 4.61(m), 4.71 (d, J=2 Hz), 6.03 (s).

### Example 57

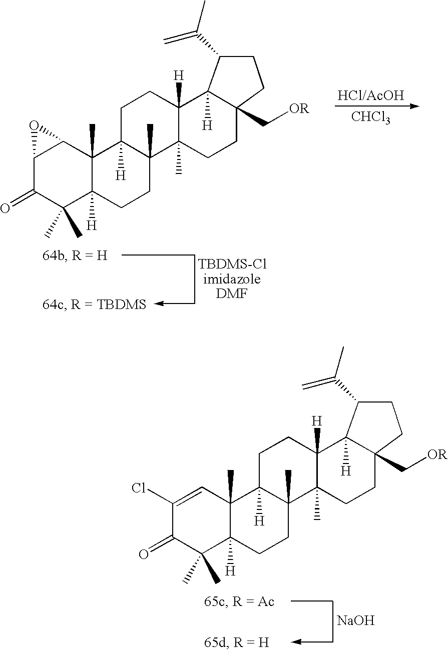

To a solution of 64b (90 mg, 0.19 mmol) in DMF (3 mL), while stirring at room temperature under N₂, were added in succession imidazole (40 mg, 0.59 mmol) and TBDMS-Cl (36 mg, 0.24 mmol). After stirring for 2 h, the reaction solution was diluted with EtOAc (10 mL) and washed with saturated NaHCO₃ (10 mL). The organic layer was separated, dried (Na₂SO₄) and concentrated under reduced pressure to a residue which was purified by silica gel column chromatography (10-50% EtOAc/hexane) to provide 97 mg (86% yield) of 64c.

A solution of 64c (50 mg, 0.09 mmol) in CHCl₃ (2 mL) was stirred at room temperature while a solution of HCl in AcOH (1 M, 2 mL, 2 mmol) was added dropwise. After 16 h, the reaction solution was diluted with CH₂Cl₂ (10 mL) and washed with saturated NaHCO₃ (10 mL). The aqueous layer was extracted with EtOAc (10 mL) and the combined organic layers were dried (Na₂SO₄) and concentrated under reduced pressure to an oily residue which was purified by silica gel column chromatography (0-20% EtOAc/hexane) to afford 45 mg of 65c.

The acetate 65c (35 mg, 0.07 mmol) in THF (1.5 mL) was treated with a solution of KOH (50 mg, 0.75 mmol) in distilled H₂O (1.5 mL) at rt for 48 h. The reaction mixture was then diluted with EtOAc (10 mL) and washed with 10% HCl (10 mL) and then saturated NaHCO₃ (10 mL). The organic layer was separated, dried (Na₂SO₄) and concentrated under reduced pressure to a residue which was purified by silica gel column chromatography to afford 18 mg (56% yield) of 65d as a white solid. ¹H NMR (500 MHz, CDCl₃) δ 0.8-2.1 (m), 0.99(s), 1.10 (s), 1.11 (s), 1.13 (s), 1.19 (s), 1.69 (d, J=0.5 Hz), 2.41 (m), 3.56 (d, J=11 Hz), 3.78 (d, J=10.5 Hz), 4.60 (m), 4.77 (s), 7.28 (s). Positive ESI-MS, m/e 473.5(M−H)⁺.

### Example 58

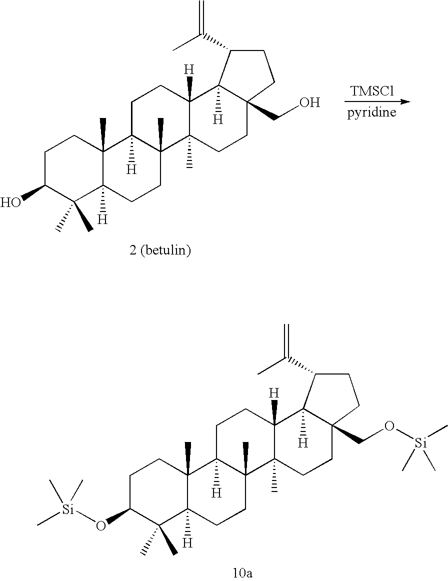

To a solution of betulin (2) (510 mg, 1.15 mmol) in dry pyridine (5 mL), while stirring at 0° C. under N₂, was added dropwise TMSCl (0.3 mL, 2.36 mmol). The heterogeneous mixture was warmed up to rt, stirred for 3 h and partitioned between H₂O (30 mL) and EtOAc (30 mL). The organic layer was washed with additional water (2×30 mL), dried (Na₂SO₄) and concentrated under reduced pressure. The residue was purified by silica gel column chromatography eluting with a gradient of 0-5% EtOAc/hexanes to provide 657 mg (97% yield) of 10a as a white solid (m.p. 132-134° C.). ¹H NMR (500 MHz, CDCl₃) δ 0.09 (s, 9H), 0.10 (s, 9H), 0.6-2.0 (m, 24 H), 0.73 (s, 3H), 0.82 (s, 3H), 0.86 (s, 3H), 0.96(s, 3H), 1.01 (s, 3H), 1.68 (s, 3H), 2.40 (m, 1H), 3.16 (dd, J=4.5, 11.5 Hz, 1H), 3.21 (d, J=10 Hz, 1H) 3.65 (d, J=9.5 Hz, 1H), 4.57 (s, 1H), 4.67 (d, J=2 Hz, 1H).

### Example 59

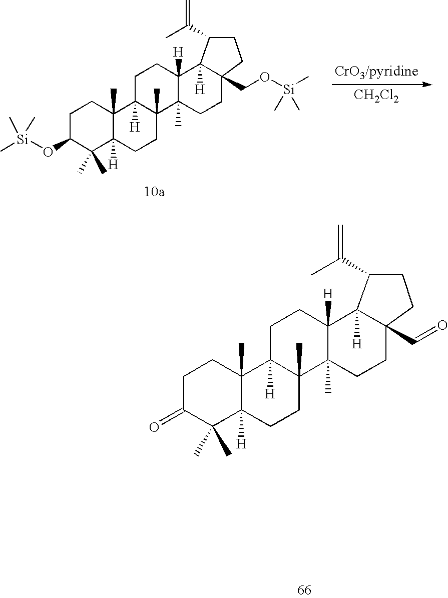

To a solution of pyridine (0.8 mL, 0.85 mmol) in CH₂Cl₂ (16 mL), while stirring at rt under N₂, was added CrO₃ (511 mg, 5.11 mmol). The resulting dark brown suspension was stirred for 0.5 h at rt and then cooled to 0° C. A solution of 10a (500 mg, 0.85 mmol) in CH₂Cl₂ (5 mL) was added dropwise. The suspension was stirred at 0° C. for an additional hour. The solids were filtered off and the filtrate was concentrated under reduced pressure. The residue was purified by silica gel column chromatography eluting with pure CH₂Cl₂ followed by 5-10% EtOAc/hexanes to afford 213 mg (57% yield) of 66 (m.p. 127-130° C.). ¹H NMR (500 MHz, CDCl₃) δ 0.80-2.2 (m, 21H), 0.92 (s, 3H), 0.95 (s, 3H), 0.98 (s, 3H), 1.02 (s, 3H), 1.07 (s, 3H), 1.70 (s, 3H), 2.46 (m, 3H), 2.88 (m, 1H), 4.63 (m, 1H), 4.76 (m, 1H), 9.67 (d, J=1.5 Hz, 1H); ¹³C NMR (500 MHz, CDCl₃) δ 14.1, 15.7, 15.9, 19.0, 19.6, 21.0, 21.2, 25.5, 26.6, 28.7, 29.1, 29.8, 33.1, 33.6, 34.1, 36.8, 38.7, 39.6, 40.7, 42.6, 47.3, 47.8, 47.9, 49.8, 54.9, 59.3, 110.2, 149.6, 206.5, 218.0.

### Example 60

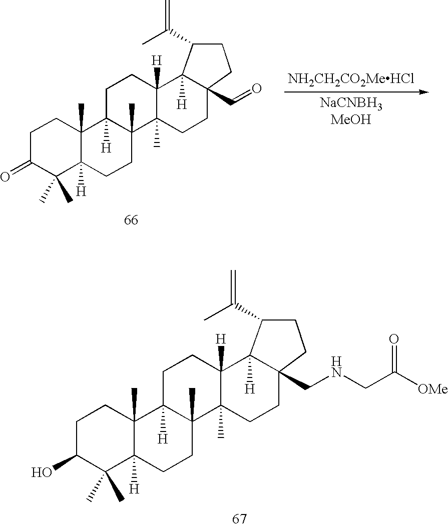

To a solution of 66 (50 mg, 0.11 mmol) in anhydrous MeOH (2 mL), while stirring at rt under N₂, was added glycine methyl ester hydrochloride (30 mg, 0.24 mmol) and NaCNBH₃ (20 mg, 0.32 mmol). After 15 min, the solution became heterogeneous and AcOH (5 drops) were added. Stirring was continued for 17 h and the reaction solution was acidified with 0.2 N HCl (3 mL) and partitioned between EtOAc (10 mL) and 1 N NaOH (10 mL). The organic layer was dried (Na₂SO₄) and concentrated under reduced pressure to a clear oil which was purified by SiO₂ column chromatography elutin with a gradient of 10-50% EtOAc/hexane to provide 30 mg (52% yield) of 67 as a white solid (m.p. 169-171° C.). ¹H NMR (500 MHz, CDCl₃) δ 0.80-2.0 (m, 26H), 0.76 (s, 3H), 0.82 (s, 3H), 0.97 (s, 6H), 1.02 (s, 3H), 1.67 (s, 3H), 2.18 (d, J=11 Hz, 1H), 2.38 (m, 1H), 2.74 (d, J=11 Hz, 1H), 3.18 (dd, J=5, 12 Hz, 1H), 3.43 (AB quartet, 2H), 3.74 (s, 3H), 4.57 (m, 1H), 4.67 (m, 1H); ¹³C NMR (500 MHz, CDCl₃) δ 14.8, 15.3, 15.8, 16.0, 18.2, 19.2, 20.8, 25.1, 27.1, 27.4, 27.9, 29.9, 30.2, 34.1, 34.9, 37.1, 38.7, 38.8, 40.9, 42.5, 46.7, 47.4, 47.5, 49.2, 50.4, 51.7, 51.8, 55.2, 78.9, 109.5, 150.6, 173.3.

### Example 61

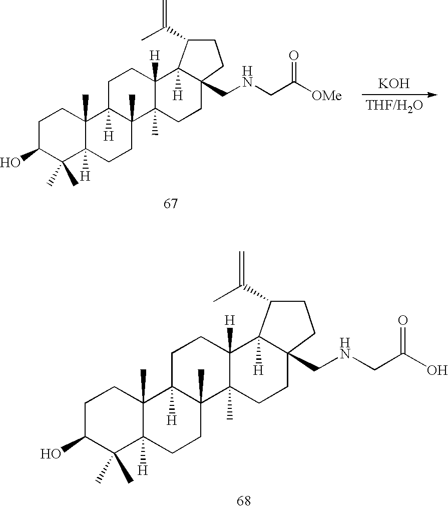

To a solution of 67 (117 mg, 0.23 mmol) in THF (2.0 mL) and CH₃OH (1.3 mL), while stirring at rt, was added dropwise an aqueous solution of NaOH (2.2 mL, 4 N, 5.5 mmol). The resulting heterogeneous mixture was stirred at rt for 48 h, diluted with H₂O (10 mL) and acidified with 4 N HCl to pH ˜4 (pH paper). After stirring for 2 h, the reaction mixture was filtered and the solids were washed with H₂O and dried under vacuum at 40° C. Further purification by silica gel column chromatography (5-10% MeOH/CH₂Cl₂) provided 54 mg (48% yield) of 68 as a white solid (m.p. >299° C.). ¹H NMR (500 MHz, CDCl₃) δ 0.68-2.2 (m, 28H), 0.75 (s, 3H), 0.86 (s, 3H), 0.95 (s, 3H), 1.02 (s, 3H), 1.09 (s, 3H), 1.70 (s, 3H), 2.45 (m, 1H), 2.83 (d, J=12.5 Hz, 1H), 3.12 (dd, J=5, 11.5 Hz, 1H), 3.52 (q, J=15.5 Hz, 2H), 4.61 (m, 1H), 4.72 (m, 1H).

### Example 62

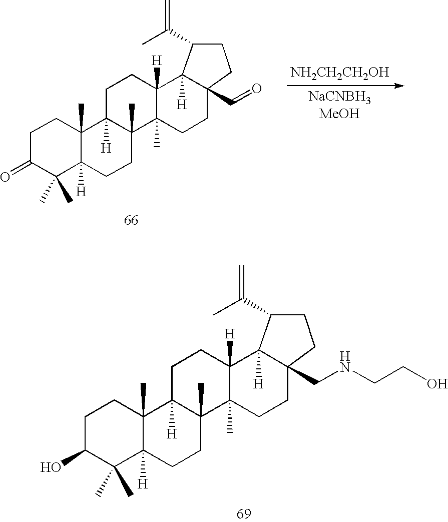

To a solution of 66 (38 mg, 0.087 mmol) in anhydrous MeOH (2 mL), while stirring at rt under N₂, were added in succession ethanolamine (11 mg, 0.18 mmol), NaCNBH₃ (15 mg, 0.24 mmol) and AcOH (5 drops). After 16 h, the reaction solution was diluted with EtOAc (10 mL) and washed with 1 N NaOH (10 mL). The organic layer was dried (Na₂SO₄) and concentrated under reduced pressure to a white solid, which was purified by SiO₂ column chromatography eluting with a gradient of 5-20% EtOAc/hexane, followed by 5-10% MeOH/CH₂Cl₂, to provide 15 mg (36% yield) of 69 as a white solid (m.p. 212-215° C.). ¹H NMR (500 MHz, CD₃OD) δ 0.70-2.1 (m, 27H), 0.75 (s, 3H), 0.86 (s, 3H), 0.95 (s, 3H), 1.02 (s, 3H), 1.09 (s, 3H), 1.70 (s, 3H), 2.47 (m, 1H), 2.53 (d, J=12.5 Hz, 1H), 2.92 (broad s, 2H), 3.02 (d, J=11.5 Hz, 1H), 3.12 (dd, J=5, 11.5 Hz, 1H), 3.75 (t, J=5.5 Hz, 2H), 4.60 (s, 1H), 4.71 (s, 1H).

### Example 63

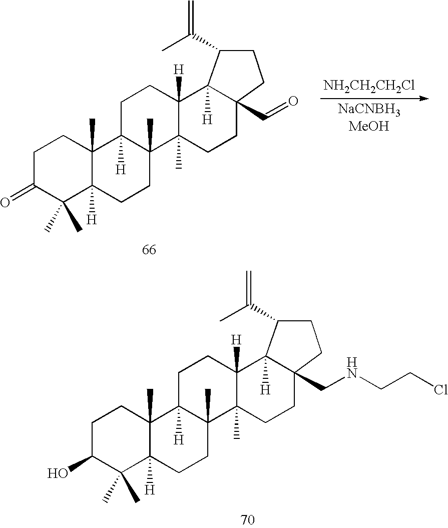

To a solution of 66 (101 mg, 0.22 mmol) in anhydrous MeOH (4 mL), whiel stirring at rt under N₂, were added in succession 2-chloroethylamine hydrochloride (55 mg, 0.48 mmol), NaCNBH₃ (40 mg, 0.64 mmol) and AcOH (5 drops). After 17 h, the reaction solution was acidified with 0.2 N HCl (4 mL) and partitioned between EtOAc (10 mL) and saturated NaHCO₃ (10 mL). The organic layer was dried (Na₂SO₄) and concentrated under reduced pressure. The residue was purification by SiO₂ column chromatography eluting with a gradient of 0-20% EtOAc/hexane to provide 76 mg (66% yield) of 70 as a white solid (m.p. 288-290° C.). ¹H NMR (500 MHz, CDCl₃) δ 0.60-2.1 (m, 26H), 0.76 (s, 3H), 0.82 (s, 3H), 0.96 (s, 3H), 0.97 (s, 3H), 1.03 (s, 3H), 1.68 (s, 3H), 2.21 (d, J=11 Hz, 1H), 2.40 (m, 1H), 2.76 (d, J=11.5 Hz, 1H), 2.98 (t, J=5.5 Hz, 2H), 3.18.(dd, J=5, 11.5 Hz, 1H), 3.68 (m, 2H), 4.57 (m, 1H), 4.68 (m, 1H).

### Example 64

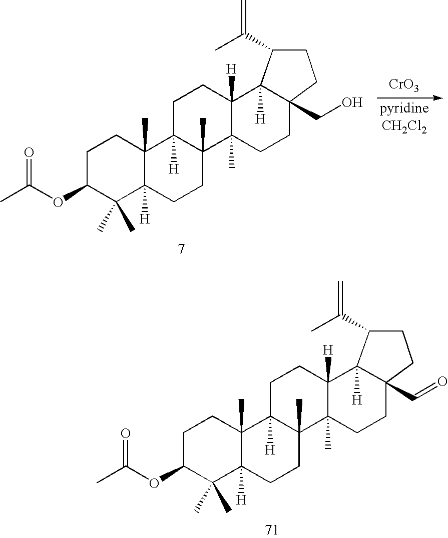

To a solution of pyridine (0.37 mL, 4.57 mmol) in CH₂Cl₂ (7 mL), while stirring at rt under N₂, was added CrO₃ (0.24 g, 2.4 mmol). The resulting dark brown suspension was stirred for 1 h at rt and then cooled to 0° C. A solution of 7 (183 mg, 0.38 mmol) in CH₂Cl₂ (3 mL) was added dropwise. The suspension was stirred at 0° C. for an additional hour. The solids were filtered off and the filtrate was concentrated under reduced pressure. The resulting residue was purified by silica gel column chromatography (5-10% EtOAc/hexane) to afford 160 mg (88% yield) of 71 (m.p. 182-184° C.). ¹H NMR (500 MHz, CDCl₃) δ 0.76-2.1 (m, 24H), 0.82 (s, 3H), 0.84 (s, 6H), 0.91 (s, 3H), 0.96 (s, 3H), 1.70 (d, J=0.5 Hz, 3H), 2.04 (s, 3H), 2.86 (m, 1H), 4.47 (m, 1H), 4.63 (m, 1H), 4.75 (d, J=1.5 Hz, 1H), 9.67 (d, J=1.5 Hz, 1H).

### Example 65

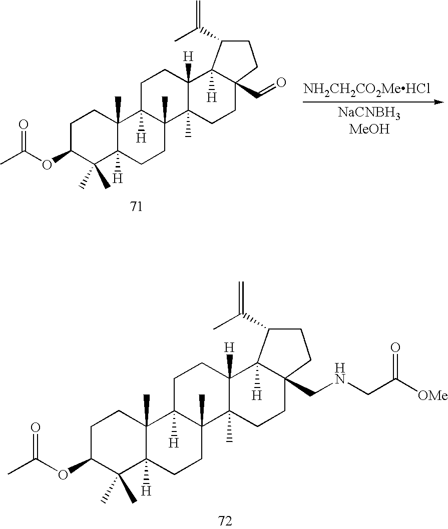

To a solution of 71 (50 mg, 0.10 mmol) in anhydrous MeOH (2 mL), while stirreding at rt under N₂, were added glycine methyl ester hydrochloride (30 mg, 0.24 mmol) and NaCNBH₃ (20 mg, 0.32 mmol). After 15 min, the solution became heterogeneous and AcOH (5 drops) was added. Stirring was continued for 17 h and the reaction solution was acidified with 0.2 N HCl (3 mL) and partitioned between EtOAc (10 mL) and 1 N NaOH (10 mL). The organic layer was dried (Na₂SO₄) and concentrated under reduced pressure to a clear oily residue, which was purified by SiO₂ column chromatography eluting with a gradient of 10-40% EtOAc/hexane to provide 14 mg (25% yield) of 72 as a white solid (m.p. 149-151° C.). ¹H NMR (500 MHz, CDCl₃) δ 0.76-2.0 (m, 26H), 0.83 (s, 3H), 0.84 (s, 6H), 0.96 (s, 3H), 1.02 (s, 3H), 1.68 (s, 3H), 2.04 (s, 3H), 2.18 (d, J=11 Hz, 1H), 2.39 (m, 1H), 2.74 (d, J=12 Hz, 1H), 3.43 (AB quartet, 2H), 3.74 (s, 3H), 4.57 (s, 1H), 4.67 (d, J=2 Hz, 1H).

### Example 66

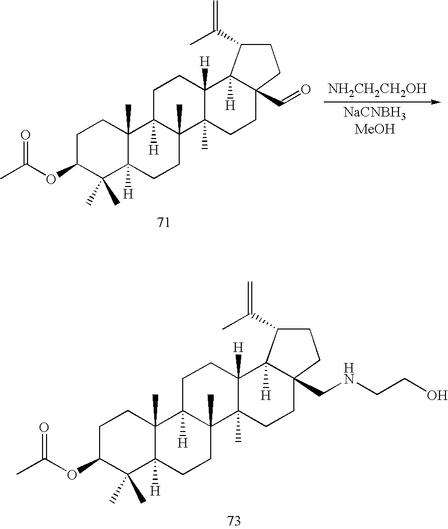

To a solution of 71 (44 mg, 0.09 mmol) in anhydrous MeOH (2 mL), while stirring at rt under N₂, were added in succession ethanolamine (12 mg, 0.19 mmol), NaCNBH₃ (16 mg, 0.26 mmol) and AcOH (5 drops). After 24 h, the reaction solution was acidified with 0.2 N HCl (3 mL) and partitioned between EtOAc (10 mL) and 1 N NaOH (10 mL). The organic layer was dried (Na₂SO₄) and concentrated under reduced pressure. The crude residue was purified by SiO₂ column chromatography eluting with a gradient of 5-20% EtOAc/hexane, followed by another gradient of 5-10% MeOH/CH₂Cl₂, to provide 28 mg (58% yield) of 73 as a white solid (m.p. 148-151° C.). ¹H NMR (500 MHz, CD₃OD) δ 0.80-2.1 (m, 26H), 0.85 (s, 3H), 0.86 (s, 3H), 0.90 (s, 3H), 1.03 (s, 3H), 1.09 (s, 3H), 1.70 (s, 3H), 2.01 (s, 3H), 2.42 (d, J=12.5 Hz, 1H), 2.47 (m, 1H), 2.83 (t, J=5.5 Hz, 2H), 2.92(d, J=11.5 Hz, 1H), 3.73 (t, J=5.5 Hz, 2H), 4.44 (dd, J=5, 11.5 Hz, 1H), 4.59 (m, 1H), 4.71 (d, J=2 Hz, 1H).

### Example 67

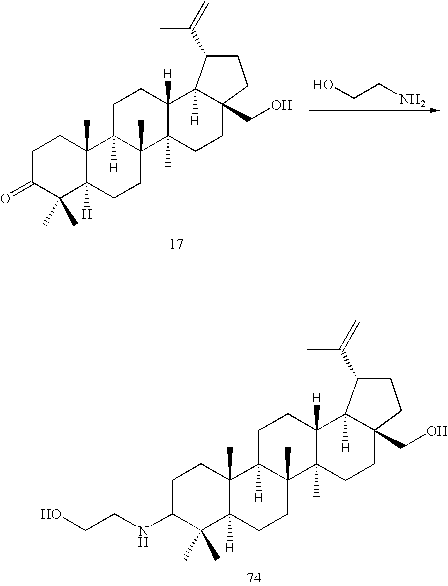

To a solution of 17 (0.2 g, 22 mmol) in methanol (5 mL), while stirring at rt under N₂, was added dropwise ethanolamine (0.04 mL, 0.68 mmol). After 30 min, NaCNBH₃ (0.05 g, 0.90 mmol) and sodium acetate (0.066 g, 1.64 mmol) were added in succession and stirring was continued for 5 days. The reaction mixture was then diluted with CH₂Cl₂ (10 mL) and washed with 10% aqueous NH₄OH (4 mL). The aqueous layer was extracted with CH₂Cl₂ (2×5 mL) and the combined organic layers were washed with brine (25 mL), dried (Na₂SO₄) and concentrated under reduced pressure to a crude solid (0.23 g). Further purification by SiO₂ column chromatography (0.2:99.4:0.4 MeOH/CH₂Cl₂/aq. NH₄OH) provided 27 mg (23% yield) of 74 (m.p. 285-293° C.). ¹H NMR (500 MHz, DMSO-d₆) δ 0.64 (s, 3H), 0.77 (s, 3H), 0.91 (s, 3H), 0.92 (s, 3H), 0.98 (s, 3H),1.63 (s, 3H), 0.64-0.85 (m), 2.37 (m), 2.77 (m, 1H), 3.08 (m, 1H), 3.43 (m, 2H), 3.52 (m, 1H), 4.21 (m, 1H), 4.53 (m, 1H), 4.66 (m, 1H).

### Example 68

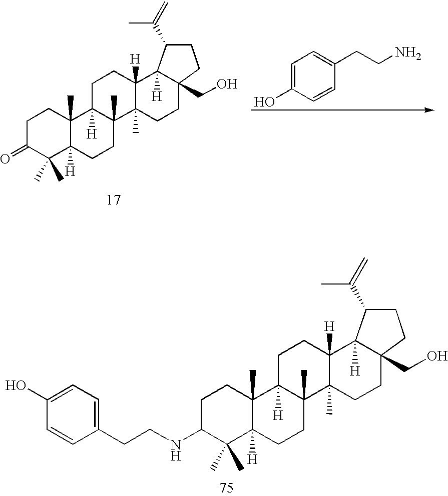

To a solution of 17 (0.2 g, 22 mmol) in methanol (5 mL), while stirring at rt, was added tyramine (0.09 g, 0.68 mmol). After 30 min, NaCNBH₃ (0.06 g, 0.90mmol) and sodium acetate (0.069 g, 85 mmol) were added in succession and stirring was continued for 5 days. The reaction mixture was diluted with CH₂Cl₂ (10 mL) and washed with 10% aqueous NH₄OH (4 mL). The aqueous layer was extracted with CH₂Cl₂ (2×5 mL) and the combined organic were washed with brine (25 mL), dried (Na₂SO₄) and concentrated under reduced pressure to a crude solid. Further purification by silica gel column chromatography (5:94.6:0.4 MeOH/CH₂Cl₂/aq. NH₄OH) provided 62 mg (25% yield) of 75 (m.p. 166-170° C.). ¹H NMR (500 MHz, DMSO-d₆) δ 0.60 (s, 3H), 0.75 (s, 3H), 0.86 (s, 3H), 0.92 (s, 3H), 0.97 (s, 3H),1.63 (s, 3H), 0.60-1.63 (m), 1.85 (m, 3H), 2.37 (m, 1H), 2.56 (m), 2.87 (m, 1H), 3.01 (m, 1H), 3.52 (m, 1H), 4.21 (m, 1H), 4.53 (s, 1H), 4.66 (m,1H), 6.65 (d, J=8.5 Hz, 2H), 6.98 (d, J=8.5 Hz, 2H), 9.08 (s, 1H).

### Example 69

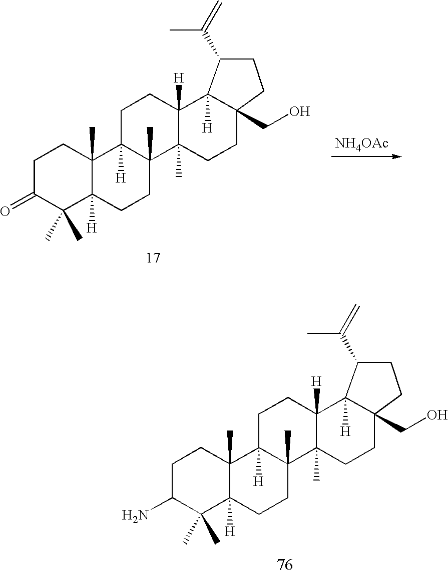

To a solution of 17 (0.2g, 0.45 mmol) in methanol (10 mL), while stirring at rt under N₂ was adde ammonium acetate (0.69 g, 9.07 mmol). The mixture was heated at 60° C. for 30 min and then cooled to rt. A solution of NaCNBH₃ (0.09 g, 1.40 mmol) in MeOH (2 mL) was added. After stirring overnight, the reaction mixture was diluted with CH₂Cl₂ (10 mL) and washed with 10% aqueous NH₄OH (4 mL). The aqueous layer was extracted with CH₂Cl₂ (2×5 mL) and the combined organic layers were washed with brine (25 mL), dried (Na₂SO₄) and concentrated under reduced pressure to a crude solid (0.19 g). Further purification by silica gel column chromatography (0.2:99.4:0.4 MeOH/CH₂Cl₂/aq. NH₄OH) provided 60 mg (30% yield) of 76 (m.p. 156-163° C.). ¹H NMR (500 MHz, DMSO-d₆) δ 0.62 (s, 3H), 0.77 (s, 3H), 0.78 (s, 3H), 0.96 (s, 3H), 0.97 (s, 3H), 1.63 (s, 3H), 0.62-1.91 (m), 2.35-2.45 (m), 3.08 (m, 1H), 3.52 (m, 1H), 4.21 (b, 1H), 4.53 (d, J=2 Hz, 1H), 4.66 (d, J=2 Hz, 1H).

### Example 70

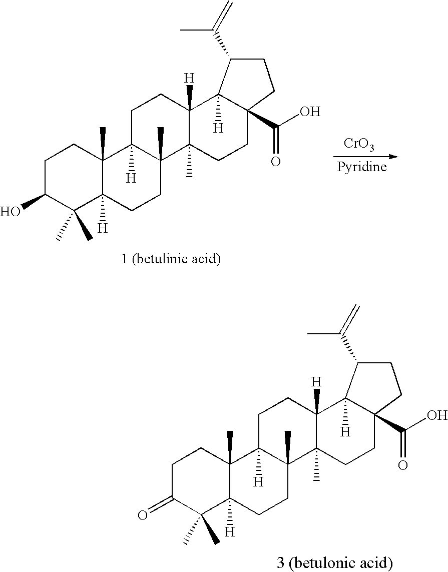

To a solution of pyridine (25.47 mL, 315.29 mmol) in CH₂Cl₂ (720 mL), while stirring at rt under N₂ was added CrO₃ (15.76 g, 157.64 mmol). The resulting dark brown suspension was stirred for 2 h at rt and then cooled to 0° C. Betulinic acid (1) (12 g, 26.27 mmol) was added portionwise. The suspension was stirred at 0° C. for additional 5.5 h. After warming up to rt. The precipitates were filtered and washed with additional CH₂Cl₂ (300 mL). The combined solutions were concentrated under reduced pressure and the crude product was purified by SiO₂ column chromatography (8:1 hexane/EtOAc) to provide 6.08 g (51% yield) of 3 (betulonic acid) (m.p. 262-267° C.). ¹H NMR (500 MHz, DMSO-d₆) δ 0.84 (s, 3H), 0.90 (s, 3H), 0.92 (s, 3H), 0.95(s, 3H), 0.98 (s, 3H), 1.65 (s, 3H), 0.84-2.50 (m, 20H), 2.94 (m, 1H), 4.56 (s, 1H), 4.69 (d, J=2 Hz, 1H), 12.07 (b, 1H).

### Example 71

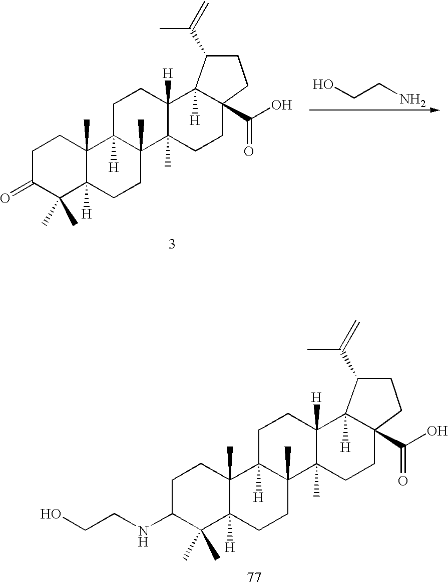

To a solution of 3 (0.2 g, 43 mmol) in methanol (5 mL), while stirring at rt under N₂, was added ethanolamine (0.08 mL, 1.31 mmol). After 30 min, NaCNBH₃ (0.11 g, 1.75 mmol) and sodium acetate (0.13 g, 1.64 mmol) were added in succession and stirring was continued for 4 days. The reaction mixture was diluted with CH₂Cl₂ (10 mL) and washed with 10% aqueous NH₄OH (4 mL). The aqueous layer was extracted with CH₂Cl₂ (2×5 mL). The combined organic layers were washed with brine (25 mL), dried (Na₂SO₄) and concentrated under reduced pressure to a crude solid (43 mg). Further purification by SiO₂ column chromatography (0.2:99.4:0.4 MeOH/CH₂Cl₂/aq. NH₄OH) provided 20 mg (9% yield) of 77 (m.p. 293-295° C.) ¹H NMR (500 MHz, DMSO-d₆) δ 0.62 (s, 3H), 0.76 (s, 3H), 0.87 (s, 3H), 0.90 (s, 3H), 0.93 (s, 3H), 1.64 (s, 3H), 0.62-0.95 (m, 24H), 1.81 (m,2H), 2.12 (d, J=10 Hz, 1H), 2.23 (m, 1H), 2.41 (m, 1H), 2.75 (m, 1H), 2.95 (m, 1H), 3.41(t, J=5 Hz, 2H), 4.56 (s, 1H), 4.69 (s, 1H).

### Example 72

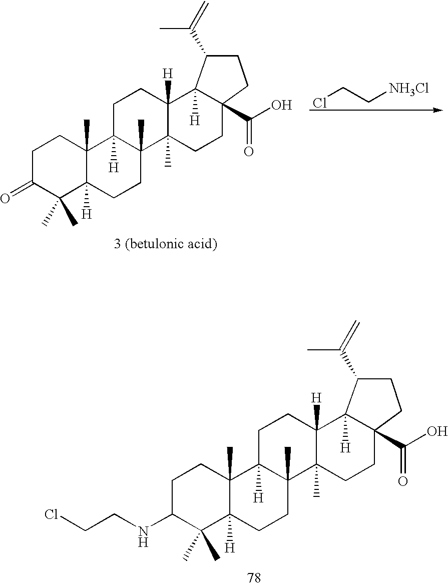

To a solution of 3 (betulonic acid) (0.2 g, 43 mmol) in methanol (5 mL), while stirring at rt under N₂, was added 2-chloroethalamine hydrochloride (0.15 g, 1.31 mmol). After 30 min, NaCNBH₃ (0.11 g, 1.75 mmol) and sodium acetate (0.13 g, 1.64 mmol) were added in succession and stirring was continued for 4 days. The reaction mixture was diluted with CH₂Cl₂ (10 mL) and washed with 10% aqueous NH₄OH (4 mL). The aqueous layer was extracted with CH₂Cl₂ (2×5 mL) and the combined organic layers were washed with brine (25 mL), dried (Na₂SO₄) and concentrated under reduced pressure to a crude solid (0.24 g). Further purification by SiO₂ column chromatography (5:94.6:0.4 MeOH/CH₂Cl₂/aq. NH₄OH) afforded 30 mg (13% yield) of 78. ¹H NMR (500 MHz, DMSO-d₆) δ 0.63 (s, 3H), 0.76 (s, 3H), 0.87 (s, 3H), 0.90 (s, 3H), 0.93 (s,3H), 0.53-1.65 (m, 24H), 1.85 (m, 2H), 2.1 (m, 1H), 2.76 (m, 1H), 2.95 (m, 1H), 3.42 (t, J=5 Hz, 2H), 4.56 (s, 1H), 4.68 (s, 1H).

### Example 73

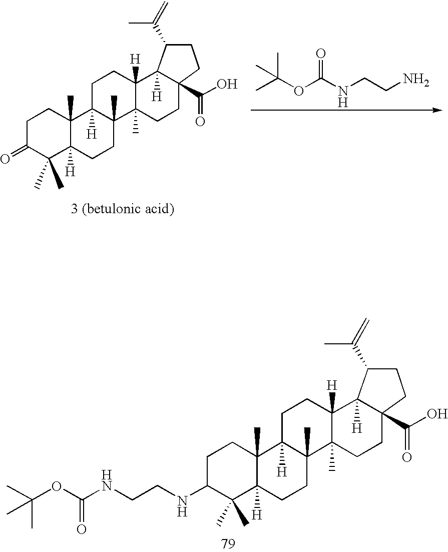

To a solution of 3 (0.2 g, 43 mmol) in methanol (5 mL), while stirring at rt under N₂, was added dropwise N-Boc-ethylenediamine (0.2 mL, 1.31 mmol). After 30 min, a solution of NaCNBH₃ (0.11 g, 1.75 mmol) in methanol (2 mL) and powder sodium acetate (0.13 g, 1.64 mmol) were added in succession and stirring was continued for 6 days. The reaction mixture was diluted with CH₂Cl₂ (10 mL) and washed with 10% aqueous NH₄OH (4 mL). The aqueous layer was extracted with CH₂Cl₂ (2×5 mL) and the combined organic layers were washed with brine (25 mL), dried (Na₂SO₄) and concentrated under reduced pressure to a crude solid (0.35 g). Further purification by SiO₂ column chromatography (0.2:99.4:0.4 MeOH/CH₂Cl₂/aq. NH₄OH) provided 126 mg (48% yield) of 79 (m.p.187-193° C). ¹H NMR (500 MHz, DMSO-d₆) δ 0.61 (s, 3H), 0.76 (s, 3H), 0.86 (s, 3H), 0.89 (s, 3H), 0.92 (s, 3H), 1.64 (s, 3H), 0.61-1.64 (m, 32H), 1.80 (m, 2H), 2.12 (m, 1H), 2.22 (m, 1H), 2.39 (m, 1H), 2.69 (m, 1H), 2.97 (m, 3H), 4.56 (s, 1H), 4.68 (s, 1H), 6.67 (m, 1H).

### Example 74

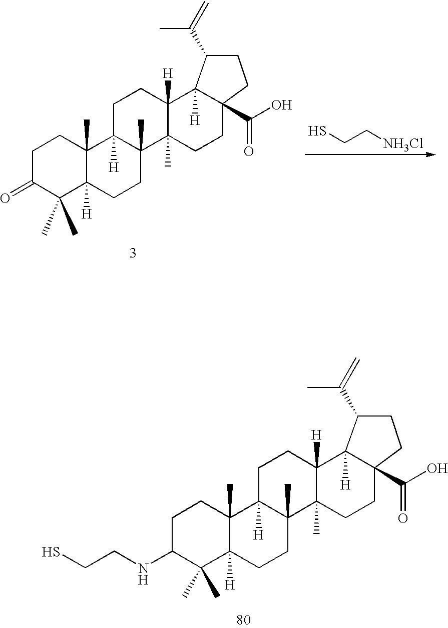

To a solution of 3 (0.2 g, 43 mmol) in methanol (5 mL), while stirring at rt under N₂, was added cysteamine hydrochloride (0.15 g, 1.31 mmol). After 30 min, a solution of NaCNBH₃ (0.11 g, 1.75 mmol) in methanol (2 mL) and powder sodium acetate (0.13 g, 1.64 mmol) were added in succession and stirring was continued for 6 days. The reaction mixture was partitioned between CH₂Cl₂ (10 mL) and 10% aqueous NH₄OH (4 mL). The solids formed were filtered, dried under reduced pressure and further purified by gradient SiO₂ column chromatography (0.2-20% MeOH/CH₂Cl₂ with 10 drops of aqueous NH₄OH/100 mL of solution) to afford 12 mg (5% yield) of 80 (m.p.284-289° C.). ¹H NMR (500 MHz, DMSO-d₆) δ 0.65 (s), 0.76 (s), 0.87 (s), 0.93 (s), 1.64 (s), 0.65-1.64 (m), 1.81 (m), 2.12 (m), 2.22 (m, 1H), 2.96 (m), 4.56 (s, 1H), 4.68 (s, 1H).

### Example 75

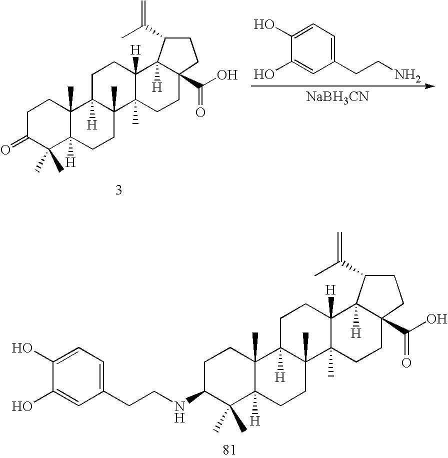

To a solution of 3 (betulonic acid) (0.15 g, 0.32 mmol) in methanol (5 mL), while stirring at rt under N₂, was added dopamine hydrochloride (0.13 g, 0.65 mmol). After 0.5 h, a solution of NaCNBH₃ (0.065 g, 1.022 mmol) in methanol (2 mL) was added. After stirring for 5 days, 10% aqueous NH₄OH (1 mL) was added and the reaction mixture concentrated under reduced pressure. The resulting residue was diluted with CH₂Cl₂ (5 mL) and washed with 5% NH₄OH (2 mL). The solids formed were filtered, washed with water (15 mL) and dried under reduced pressure at 45° C. The crude solid was triturated with hot ethanol (3×3 mL) and the ethanolic solutions were combined and concentrated under reduced pressure to afford 55 mg (28% yield) of 81 (m.p. 240-250° C.). ¹H NMR (500 MHz,DMSO-d₆) δ 0.064 (s, 3H), 0.76 (s, 3H), 0.86 (s, 3H), 0.92 (s,3H), 1.04 (s, 3H), 1.64 (s, 3H), 0.59-1.87 (m), 2.15 (m, 2H), 2.26 (m, 2H), 2.74 (m, 1H), 2.82 (m, 1H), 2.96 (m), 3.43 (m), 3.77 (m), 4.32 (m, 1H), 4.55 (s, 1H), 4.68 (s, 1H)6.28-6.45 (m, 2H),6.56-6.63 (m, 2H). D₂O Exchange NMR (500 MHz, DMSO-d₆) δ 0.65 (s, 3H), 0.76 (s, 3H), 0.86 (s, 3H), 0.92 (s, 3H), 1.05 (s, 3H), 1.64 (s, 3H), 0.60-1.80 (m), 2.12 (m, 1H), 2.47(m, 2H), 2.57-2.65 (m), 2.79 (m, 1H), 2.84 (m, 1H), 2.96 (m), 3.77 (m, 1H), 4.55 (s, 1H), 4.67 (s, 1H), 6.45 (m, 2H), 6.63 (m, 2H). ¹³C NMR (500 MHz, DMSO-d₆), δ 15.79, 18.92 25.08, 25.45, 26.36, 27.12, 28.06, 29.18, 30.11, 33.05, 33.58, 33.90, 36.32, 36.72,37.53, 38.23, 38.45, 41.96, 42.41, 46.48, 48.54, 50.07, 53.80, 54.85, 55.44, 56.07,61.96, 65.36, 76.74, 109.52, 115.95, 116.03, 119.14, 131.16,143.66, 145.18, 150.39, 177.38. MS (ESI+) m/e 593 (M+H).

### Example 76

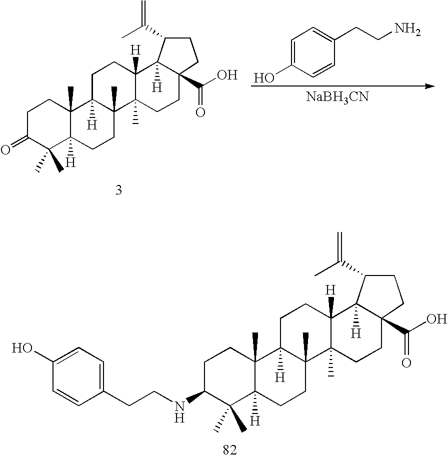

To a solution of 3 (0.20 g, 0.43 mmol) in methanol (5 mL), while stirring at rt under N₂, was added tyramine (0.18 g, 1.31 mmol). After 0.5 h, a solution of NaCNBH₃ (0.11 g, 1.75 mmol) in methanol (2 mL) was added. After stirring for 5 days, 10% aqueous NH₄OH solution (1 mL) was added and the reaction mixture was concentrated under reduced pressure. The resulting residue was diluted with CH₂Cl₂ (5 mL) and washed with 5% NH₄OH (2 mL). The solids formed were filtered, washed with water (10 mL), CH₂Cl₂ (10 mL) and dried under reduced pressure at 45° C. The crude solid was dissolved in hot ethanol (3 mL) and re-precipitated upon addition of cold water (3 mL). After being washed with water (5 mL) and dried under reduced pressure, the solid was recrystalization from hot MeOH (4×5 mL) and cold water (5 mL) to afford 48 mg (19% yield) of 82 (m.p. 204-210° C.). ¹H NMR (400 MHz,DMSO-d₆) δ 0.59 (s, 3H), 0.75 (s, 3H), 0.86 (s, 6H), 0.92 (s, 3H)1.64 (s, 3H), 0.59-1.80 (m), 2.12 (m, 1H), 2.22 (m, 1H), 2.53 (m), 2.88 (m, 1H), 2.94 (m, 1H), 4.55 (s, 1H), 4.68 (s, 1H), 6.65 (d, J=8.4 MHz, 2H), 6.99 (d, J=12 MHz, 2H). MS (APCI+) m/e 577 (M+H); MS (ESI+) m/e 577 (M+H).

### Example 77

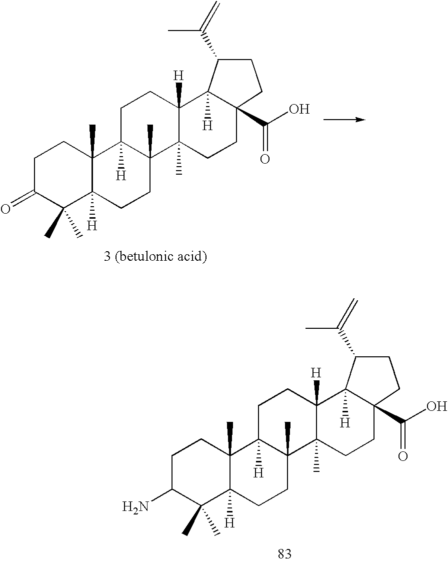

To a solution of 3 (3.5 g, 7.69 mmol) in methanol (150 mL), while stirring at rt under N₂, was added ammonium acetate (11.87 g, 153.94 mmol). The reaction mixture was heated at 60° C. for 5 h and then cooled to rt. A solution of NaCNBH₃ (1.5 g, 23.86 mmol) in MeOH (25 mL) was added dropwise. Stirring was continued overnight and 10% aqueous NH₄OH (100 mL) was added. The heterogenous mixture was concentrated under reduced pressure to approximate half in valume and partitioned between CH₂Cl₂ (500 mL) and water (400 mL). The white solid formed was filtered, washed with water and dried under high vacuum at 40° C. overnight to afford 3.24 g of a crude material. Further purification by SiO₂ column chromatography eluting with a gradient of 10-20% MeOH/CH₂Cl₂ containing 10 drops of aqueous NH₄OH/100 mL of solution provided 2.10 g (60% yield) of 83. ¹H NMR (500 MHz,CDCl₃) δ 0.76 (s, 3H), 0.82 (s, 3H), 0.95 (s, 3H), 0.96 (s, 6H), 1.67 (s, 3H), 0.76-1.72 (m, 22H), 1.90 (m, 3H), 2.22 (m, 1H), 2.36 (m, 1H), 2.51 (m, 1H), 3.04 (m, 1H), 4.55 (m, 1H), 4.69 (m, 1H). ¹³C NMR (500 MHz, CDCl₃) o 15.09, 15.89, 16.44, 16.56, 19.06, 19.72, 21.51, 26.04, 26.23, 28.35, 30.36, 31.38, 33.35, 34.94, 37.86, 38.07, 38.81, 39.52, 41.25, 43.09, 47.72, 49.96, 51.31, 56.50, 57.41, 60.23, 109.65, 151.85, 181.63. MS (ESI+) m/e 457 (M+H).

### Example 78

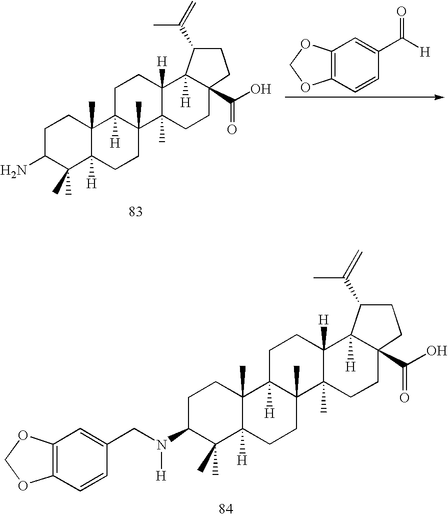

To a solution of 83 (100 mg, 0.21 mmol) in MeOH (5 mL) and THF (30 mL), while stirring at rt under N₂, was added dropwise piperonal (100 mg, 0.65 mmol). After 1 h, a solution of NaCNBH₃ (60 mg, 0.87 mmol) in MeOH (2 mL) and powder sodium acetate (70 mg, 0.82 mmol) were added in succession. Stirring was continued for 4 days and CH₂Cl₂ (25 mL) was added. The reaction mixture was washed with 10% aqueous NH₄OH (15 mL), water (2×10 mL) and brine (10 mL). The organic layer was dried (Na₂SO₄) and concentrated under reduced pressure to a crude product. Further purification by SiO₂ column chromatography (0.2:99.4:0.4 MeOH/CH₂Cl₂/aq. NH₄OH) afforded 35 mg (27% yield) of 84 (m.p. 294-297° C.). ¹H NMR (500 MHz, DMSO-d₆) δ 0.66 (s, 3H), 0.76 (s, 3H), 0.86 (s, 6H), 0.90 (s, 3H), 1.63 (s, 3H), 0.66-1.79 (m, 25H), 2.11 (m, 1H), 2.21 (m, 1H), 2.94 (m, 1H), 3.50 (b, 1H), 3.78 (b, 1H), 4.55 (s, 1H), 4.68 (s, 1H), 5.97 (s, 2H), 6.93 (bs, 3H).

### Example 79

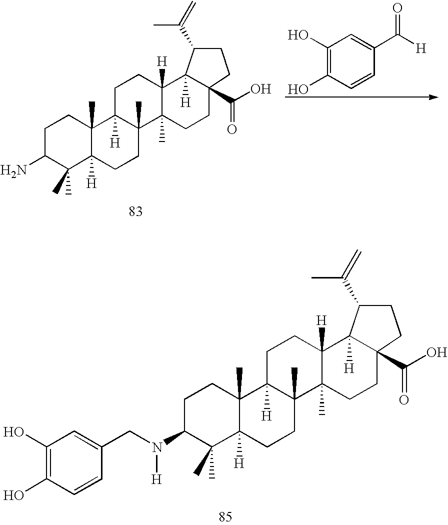

To a solution of 83 (100 mg, 0.21 mmol) in MeOH (2 mL) and THF (10 mL), while stirring at rt under N₂, was added 3,4-dihydrobenzaldehyde (90 mg, 0.65 mmol). After 0.5 h, a solution of NaCNBH₃ (60 mg, 0.87 mmol) in MeOH (2 mL) and sodium acetate (70 mg, 0.82 mmol) were added in succession. Stirring was continued for 2 days and CH₂Cl₂ (25 mL) added. The reaction mixture was washed with 10% NH₄OH (15 mL), water (2×10 mL) and brine (10 mL). The organic layer was dried (Na₂SO₄) and concentrated under reduced pressure. The crude material was purified by SiO₂ column chromatography (10:89.6:0.4 MeOH:CH₂Cl₂:aq. NH₄OH) to afford 20 mg (15% yield) of 85 (m.p. 228-232° C.). ¹H NMR (500 MHz,DMSO-d₆): δ 0.63 (s 3H), 0.76 (s, 3H), 0.87 (s, 3H), 0.91 (s, 3H), 0.93 (s, 3H), 1.64 (s, 3H), 0.63-1.90 (m), 2.11 (m), 2.22 (m), 2.40 (m), 2.71 (m), 2.95 (m), 3.31-3.41 (m), 4.56 (s, 1H), 4.68 (s, 1H).

### Example 80

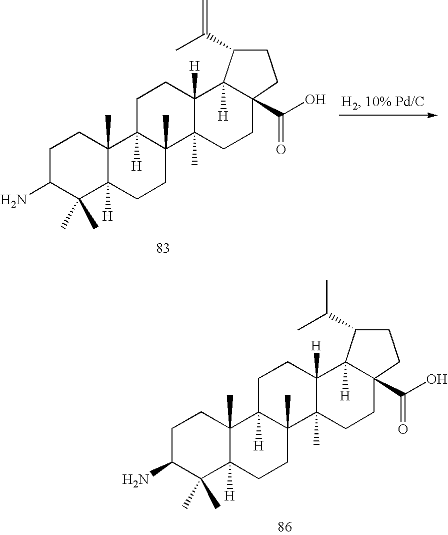

A suspension of 83 (0.075 g, 0.16 mmol) and Pd/C (10% wt on activated carbon, 0.03 g) in CH₂Cl₂ (10 mL) and MeOH (5 mL) was hydrogenated at 30 psi for 72 h. The reaction mixture was then filtered through Celite and washed with 2:1 mixture of CH₂Cl₂ and MeOH (100 mL). The combined filtrates were concentrated under reduced pressure to a solid, which was further purified by SiO₂ column chromatography (20% CH₂Cl₂/MeOH) to give 45 mg (60% yield) of 86. ¹H NMR (500 MHz, DMSO-d₆) δ 0.72 (s, 3H), 0.73 (d, J=6 Hz, 3H), 0.77 (s, 3H), 0.82 (d, J=7 Hz, 3H), 0.87 (s, 3H), 0.91 (s, 3H), 0.96 (s, 3H), 0.70-1.80 (m, 23H), 2.12 (m, 2H), 2.26 (m, 1H), 2.63 dd, J=4.5 and 11.5 Hz, 1H), 3.3 (bs, 2H).

### Example 81

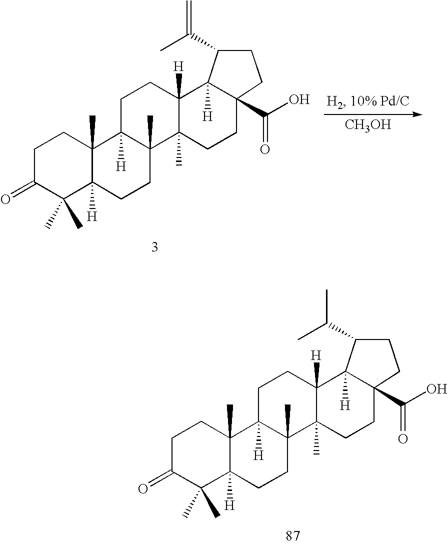

To a solution of 3 (0.3 g, 0.65 mmol) in MeOH (15 mL) and Pd/C (10% wt on activated carbon, 0.1 g) in MeOH (15 mL) was hydrogenated at 30 psi for 16 h. The reaction mixture was filtered through Celite and washed with MeOH (100 mL). The filtrate was concentrated under reduced pressure to a crude solid, which was purified by silica gel column chromatography (2% MeOH/CH₂Cl₂) to give 70 mg (23% yield) of 87 (258-265° C.). ¹H NMR (500 MHz, CDCl₃) δ 0.76 (d, J=6.5 Hz, 3H), 0.86 (d, J=6.5 Hz, 3H), 0.93 (s, 3H), 0.97 (d, J=2 Hz, 6 H), 1.02 (s, 3H), 1.07 (s, 3H), 0.75-1.94 (m, 25 H), 2.25 (m, 1H) 2.48 (m, 1H).

### BIOLOGICAL ASSAYS

### Biological Example 1

### MTS Cell Viability Assay—Betulinic Acid Analogs

Cells were plated, the evening before treatment, for each treatment on 96-well plates (1×10⁴ cells/well) in 100 μL volume per well. Drug solutions were prepared by diluting each test compound in DSMO (10 mM) with the appropriate cell growth media for each individual cell line, or in “Universal Media” if several cell lines were to be assayed in parallel, immediately prior to cell treatment. Universal Media consisted of 5 mL sodium pyruvate (100×liquid stock, CellGro), 5 mL glucose (100×, 45% liquid stock, CellGro), 5 mL Penicillin/Streptomycin (100×liquid stock, CellGro), 10 mL sodium bicarbonate (50×liquid stock, CellGro), 25 mL Fetal Calf Serum, 1.25 mL insulin (4 mg/mL, Gibco) and 449 mL RPMI media with 2 mM glutamine for a total volume of 500 mL. Cells were treated by aspirating media from each well and adding 80 μL of each drug solution to the attached cells. All treatments were performed in triplicate. Growth media (80 μL) were added to 3 blank wells (no cells) to measure background from the growth media Growth media alone (no DMSO or test compond) was added to 2 wells containing cells to measure the baseline MTS activity and vehicle (DMSO) control solutions were also included to monitor basal toxicity from DMSO. Cells were incubated at 37° C. for 72 h. MTS reagent (per 96-well plate) were prepared by combining 2 mL of MTS working solution (Cell Titer AQueous Non-Radioactive Cell Proliferation Assay, Promega, cat#G1112), 100 μL of 0.92 mg/mL phenazine methosulfate/Dulbecco's PBS and 2.1 mL growth media. MTS reagent (40 μL) were added to each well and incubated at 37° C. for 1.5 to 4 h. Plates were gently shaken by hand until solution in each well appeared homogenous. Absorbances at 490 nm were measured on a plate reader at multiple time points following the addition of MTS reagent for each plate. Triplicate absorbance (490 nm) measurements were averaged following background (no cell) subtraction for each drug concentration. Percent Cell Viability was calculated for each drug concentration using the following equation:
{[Absorbances(drug treated)]/[Absorbances(DMSO treated)]}×100%

Percent viability (y-axis) was plotted against drug concentration (x-axis) and the resulting graph was used to determine the 50% inhibitory concentration (IC₅₀) for each drug.

### Biological Example 2

### Caspase Assay—Betulinic Acid Analogs

Cells were plated, the evening before treatment, for each treatment (1×10⁴ cells/well) on black-walled, clear-bottomed, 96-well plate, in 100 μL volume per well. Drug solutions were prepared, immediately prior to cell treatment, by diluting each test compound in the appropriate cell growth media without fetal calf serum (FCS) or in “Universal Media” without FCS if several cell lines were to be assayed in parallel.

Cells were treated by aspirating media from each well and adding 70 μL of each drug solution to the attached cells. All treatments were performed in duplicate. Growth media (70 μL) was added to 2 blank wells (no cells) to measure background from the growth media. Growth media alone (no DMSO or test compond) was added to 2 wells containing cells to measure the baseline fluorescence and vehicle (DMSO) control solutions were also included to monitor basal caspase induction from DMSO. Cells were incubated at 37° C. for 8 h. Caspase assay reagent (per 96-well plate) was prepared according to manufacturer's instructions (Homogeneous Caspases Assay, fluorometric, Roche) by combining 6.3 mL Incubation Buffer with 0.7 mL of Substrate Stock Solution. Caspase assay reagent (70 μL) was added to each well; the plate was gently shaken by hand for 15-20 seconds and incubated at 37° C. for 4 h.

Fluorescent emission was measured at 535 nm on a plate reader using the “homogeneous caspase” program (excitation Wavelength=490 nm, emission wavelength=535 nm). Duplicate wells for each treatment were averaged following background (no cells) subtraction (emission 535 nm value from all experimental emission 535 nm values) for each drug concentration. Percent change in caspase activity was calculated for each drug concentration using the following equation:
{{[Emission₅₃₅(drug treated)]−[Emission₅₃₅(DMSO treated)]}/Emission₅₃₅(DMSO treated)}×100%

DMSO treatment represented baseline caspase activation in the absence of drug. The percent changes in caspase activity were plotted on the y-axis for each drug treatment.

### Biological Example 3

### Annexin-V Assay—Betulinic Acid Analogs

Cells were plated (8.75×10⁵ cells/6 cm. diameter tissue culture plates), the evening before treatment, in 4 mL volume per plate. This cell density is equivalent to the cell density used in the MTS and Caspase assays (1×10⁴ cells/well (96-well plate). Drug solutions were prepared, immediately prior to cell treatment, in the same as described for caspase assay

Cells were treated by aspirating media from each well and adding 3 mL of each drug solution to the attached cells. Growth media alone (no DMSO or drug) was added to a plate containing cells to measure the baseline-Annexin-V reactivity and vehicle (DMSO) alone control solutions were also prepared to monitor Annexin-V reactivity from DMSO. Cells were incubated at 37° C. for 8 h. Growth media (3 mL) was removed from each plate and added to a 15 mL conical tube containing 0.333 mL FCS (final FCS concentration of 10%). The media was saved to include any apoptotic/dead cells that may have detached from the plate during drug treatment. FCS was added to the media to prevent further cell damage and improve the efficiency of cell pelleting during subsequent centrifugation steps (empirical observation). Adherent cells were rinsed once with PBS and 1 mL of trypsin was added. Plates were rotated several times to assure coating of the entire surface with trypsin which was then removed. Plates were incubated at 37° C. for 4-5 min. Trypsinized cells were re-suspended in the saved media for each sample. Cell suspension was placed back into 15 mL tubes, which were then cooled on ice. Cells were re-suspended by pipetting 7-8 times. The tubes were centrifuged at 130×g for 5 min at 4° C. The resulting cell pellets were re-suspended in the ice cold 1 mL of 1× Nexin Buffer (Guava Nexin kit, Guava Technologies) and transferred to 1.5 mL conical microcentrifuge tubes to rinse cells with residual growth media. This procedure was repeated by centrifugation of cells at 130×g for 5 min, at 4° C. Re-suspension of the resulting cell pellet in 50 μL Nexin Staining Solution (Guava Nexin kit, Guava Technologies) was followed by incubation on ice, in the dark, for 20 min. Guava Samples were analyzed immediately on the Guava flow cytometer, using the Guava Nexin software package (see Guava user's manual and Guava Nexin kit protocol on data acquisition and analysis protocols).

### Biological Example 4

### Cytotoxicity Dose Response

Cytoxicity dose response for the triterpenoid derivatives synthesized in SK-MEL-2 (melanoma), A-375 (melanoma), Daoy (glioblastoma), LN-229 (glioblastoma), OVCAR-3 (ovarian carcinoma), HT-29 (colon carcinoma), MCF-7 (breast carcinoma) cell lines using the standard MTS assay is summarized in Table 2. Data revealed that among all analogs, N-hydroxy isoxazole 32, cyano keto alcohol 38, 28-amino alcohol 69, 3-tyramine 82 and 3-amino 86 were potent across the entire cell line panel whereas bromoacetyl analogs were selectively active in Daoy (glioblastoma) in general. The most effective analogs had an IC₅₀ value in the 1-6 μM range. Analogs which demonstrated greater than 5-fold improvement in cytotoxicity relative to betulinic acid 1 in Daoy (glioblastoma) included keto aldehyde 66, amino diol 69 and amino ester 72. Cafeic derivatives (24, 25 and 29) and 28-aza analogs (69, 72, and 73) demonstrated greater than 5-fold improvement in SK-MEL-2 (melanoma). Keto aldehde 66 appeared to exhibit selective toxicity towards Daoy.

TABLE 2
Biological Activities of Betulinic Acid Analogs Measured as IC₅₀ (μg/mL)

| Compound | SK-MEL-2 | A-375 | Daoy | LN-229 | OVCAR-3 | HT-29 | MCF-7 |
|---|---|---|---|---|---|---|---|
| 1 | 33.22 ± 3.77 | 51 | 31.27 ± 2.28 | 61 | 59.0 ± 4.58 | 37 | 50 |
| 13 | 33 | 29 | 1.4 | 6 | 6 | 10 | 45 |
| 18 | 42 | 14 | <2 | — | 8 | 36 | 48 |
| 19 | 6 | 7 | 7 | >75 | >75 | >75 | >75 |
| 32 | 5 | 6 | 6 | 6 | 6 | 8 | 6 |
| 38 | 6 | 6 | 8 | 6 | 7 | 8 | — |
| 39 | 20 | 4 | <2 | 7 | 7 | 6 | 13 |
| 40 | >10 | 6 | 4 | 28 | 10 | 10 | 30 |
| 49 | 6 | 10 | 4 | >75 | 50-75 | 50-75 | >75 |
| 50 | >75 | 25 | 4 | 48 | 42 | 40 | 50 |
| 55 | — | 4 | — | — | 8 | 7 | >75 |
| 66 | 28 | 15 | 6 | 35 | 20 | 21 | 38 |
| 69 | 5 | 4 | <2 | — | <10 | 2 | <10 |
| 70 | 35 | 20 | <2 | — | 10 | 19 | 54 |
| 72 | 6 | 6 | 6 | >75 | 36 | >75 | 75 |
| 73 | 5 | 4 | <2 | — | <10 | <10 | <10 |
| 77 | 36 | 14 | <2 | — | 28 | 16 | 35 |
| 82 | 5 | 5 | 5 | 5 | 6 | 4 | 6 |
| 86 | 6 | 5 | 6 | 5 | 6 | 5 | 6 |
| 87 | 16 | 22 | 5 | 28 | 22 | 35 | 38 |

Caspase activation assays were conducted to further characterize the mode of cell death. A treatment time and dose at which maximum caspase activation was observed prior to massive cell destruction would be indicative of apoptosis induction. SK-MEL-2 cells were treated with 5, 15, and 50 μM of the compound for 2, 4, 6, 8, 16, and 24 h in the absence of FBS (Fetal Bovine Serum). It is demonstrated that caspase activation induced by a triterpenoid compound peaked at around 8 h following the treatment of cancer cells with 15 and 50 μM of concentrations. N-Hydroxyisoxazole 32, cyano keto alcohol 38 and tyramine 75 had more robust caspase activating property than betulinic acid (1).

N-Hydroxyisoxazole 33 and, particularly, the A-ring modified bromoacetyl 39 induced caspase activation in SK-MEL-2. 3-β-Hydroxy bromoacetyl 13 induced caspase activation (approx. 200%) in Daoy and appeared to selectively activate apoptosis in Daoy in preference to SK-MEL-2. It is also worth noting that in the MTS assay, this analog showed robust cytotoxic activity in Daoy but not in SK-MEL-2. Cyano keto alcohol 38 activated caspases in SK-MEL-2, inducing apoptosis over necrosis robustly as evidenced by the Annexin assay. This analog therefore appears to be the most potent apoptosis inducer of this collection of compounds. N-hydroxyisoxazole 32, tyramine 75, and 3-aza 79 induced selective apoptosis at an optimal dose, above which cellular damage and non-apoptotic cell death occurred.

### REFERENCES

1. Hayek, E. W. H.; Moche, W.; Sauter, F. A Bicentenial of Betulin. Phytochemistry, 1989, 28, 2229-2242.
2. Kim, D. S. H. L; Chen, Z.; Nguyen, V. T.; Pezzuto, J. M.; Qui, S.; Lu, Z-Z. A Concise Semi-synthetic Approach to Betulinic Acid from Betulin. Synth. Commun. 1997, 27, 1607.
3. For recent reviews see: (a) Eiznhamer, D.; Xu, Z-Q. Betulinic Acid: A Promising Anticancer Candidate, IDrugs, 2004, 7, 359-373. (b) Cichewicz, R. H.; Kouzi, S. Chemistry, Biological Activity and Chemotherapeutic Potential of Betulinic Acid for the Prevention and Treatment of Cancer and HIV Infection. Med. Res. Rev.; 2004, 24, 90-114.
4. Pisha, E.; Chai, H.; Lee, I. S.; Chagwedra, T. E.; Fransworth, N. R.; Cordell, A. C.; Beecher, C. W. W:; Fong, H. H. S.; Kinghom, A. D.; Brown, D. M.; Wani, M. C.; Wall, M. E.; Hieken, T>J.; Das Gupta, T. K.; Pezzuto, J. M. Discovery of Betulinic Acid as a Selective Inhibitor of Human Melanoma that Functions by Induction of Apoptosis. Nat. Med. 1995, 1, 1046-1051.
5. (a) Fulda, S.; Jeremias, I.; Steiner, H. H.; Pietsch, T.; Debatin, K. M. Betulinic Acid: A New Cytotoxic Agent Against Malignant Brain-tumor Cells. Int. J Cancer 1999, 82, 435-441. (b) Schmidt, M. L.; Kuzmanoff, K. L.; Ling-Indeck L, Pezzuto, J. M. Betulinic Acid Induces Apoptosis in Human Neuroblastoma Cell Lines. Eur. J Cancer 1997, 33, 2007-2010. (c) Zuco, V.; Supino, R.; Righetti, S. C.; Cleris, K.; Marchesi, E.; Gambacorti-Passerini, C. Formelli, F. Selective Cytotoxicity of Betulinic Acid on Tumor Cell Lines, but not Normal Cells. Cancer lett. 2002, 175, 17-25.
6. Wick, W.; Grimmel, C.; Wagenknecht, B.; Dichgans, J.; Weller, M. Betulinic Acid-induced Apoptosis in Glioma Cells: A Sequential Requirement for New Protein Synthesis, Formation of Reactive Oxygen species, and Caspase Activity, J. Pharmacol. Exp. Ther. 1999, 293, 1306-1312.
7. Selzer, E.; Pimentel, E.; Wacheck, V.; Schlegel, W.; Pehamberger, H.; Jansen, B., Kodym, R. Effects of Betulinic Acid Alone and in Combunation with Irradiation in Human Melanoma Cells. J. Invest. Dermatol. 2000, 114, 935-940.
8. Fulda, S.; Friesen, C.; Los, M.; Scaffidi, C.; Mier, W.; Benedict, M.; Nunez G.; Krammer, P. H.; Peter, M. E.; Debatin, K. M. Betulinic Acid Triggers CD95 (APO-1/Fas)- and p53-Independent Apoptosis via Activation of Caspases in Neuroectodermal Tumors. Cancer Res. 1997, 57, 4956-4964.
9. (a) Fulda, S.; Scaffidi, C.; Susin, S. A.; Krammer, P. H.; Kroemer, G.; Peter, M. E.; Debatin, K. M. Activation of Mitochondria and Release of Mitochondrial Apoptogenic Factors by Betulinic Acid. J. Biol. Chem. 1998, 273, 33942-33948. (b) Fulda, S.; Debatin, K. M. Betulinic Acid Induces Apoptosis Through a Direct Effect on Mitochondria in Neuroectodermal Tumors. Med. Pediatr. Oncol. 2000, 35, 616-618.
10. Fulda, S.; Debatin, K-M. Sensitization for Anticancer Drug-Induced Apoptosis by Betulinic Acid. Neoplasia 2005, 7, 162-170.
11. Siddiqui, S.; Hafeez, F.; Begum, S.; Siddiqui, B. S. Oleanderol, A new Pentacyclic Triterpene From the Leaves of Nerium Oleander. J. Nat. Prod. 1988, 51, 229-233.
12. You, Y.-J.; Kim, Y.; Nam, N.-H.; Ahn, B.-Z. Synthesis and Cytotoxic Activity of A-Ring Modified Betulinic Acid Derivatives. Bioorg. Med. Chem. Lett. 2003, 13, 3137-3140.

What is claimed is:

1. A compound of the formula 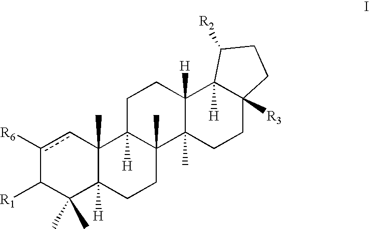
or a pharmaceutically acceptable salt thereof, wherein [symbol]
is a single or double bond;
R₁ is H, halo, NH₂, OH, SH, ═O, ═S, ═N—OH, NHR₄, NH(CH₂)ₙR₄, NR₄R₅, OR₄, OCOR₄, OC(O)OR₄, OC(O)NR₄R₅, SR₄, SCOR₄, SC(O)NR₄R₅, SC(O)NR₄R₅, NHCOR₄, NHC(O)OR₄, N(R₅)C(O)OR₄, NHC(O)NR₄R₅, N(R₅)C(O)NR₄R₅, ═N—OR₄, ═N—OCOR₄, OCO(HC═CH)ₙR₄, OCO(CH₂)ₙX, OSO₂(CH₂)ₙX, OSi(R₄)ₙ(R₅)₃₋ₙ, or SCO(CH₂)ₙX;
R₂ is C(CH₃)₂ or C(═CH₂)CH₃;
R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)R₄, CH₂OCO(CH₂)ₙX, CH₂NH₂, CH₂NHR₄, CH₂N(CH₂)ₙR₄R₅, CH₂NR₄R₅, CO₂R₄, C(O)NHR_{4,} or C(O)NR₄R₅;
R₄ and R₅ are independently H, C(O)X, halo, C₁₋₈ alkyl, aryl-C₁₋₈ alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;
R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;
R₆ is H, halo, R₄, Se-aryl, OR₄, CN, CHO, CO₂R₄, or C(R₄)ₙ(R₅)₃₋ₙ, or R₆ together with the ring to which it is attached form 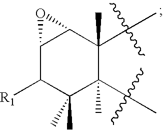
X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and
n=1-5;
provided that when R₁ is oxo, [symbol] is a double bond, R₂ is C(CH₃)₂ and R₃ is CO₂H, R₆ cannot be CN, Cl or CHO; and when R₁ is oxo, [symbol] is a double bond, R₂ is C(CH₃)₂ and R₃ is CO₂Me, R₆ cannot be CN, OMe or CHO.

2. A compound according to claim 1, wherein R₁ is OSi(CH₃)₂-tert-butyl, OSi(CH₃)₃, OH, ═O, O—C(O)—CH₃, OCO(CH₂)OCH₃, OCO(HC═CH)phenyl wherein the phenyl is substituted with two OCOCH₃ or OH, NH(CH₂)₂—OH, NH(CH₂)₂—Cl, NH(CH₂)₂—SH, NH(CH₂)-phenyl or NH(CH₂)₂-phenyl wherein the phenyl is substituted with OH, NH₂, O-pyranyl, NH(CH₂)₂—NHC(O)O-tert-butyl or NH(CH₂)benzodioxolyl.

3. A compound according to claim 1, wherein R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

4. A compound of the formula 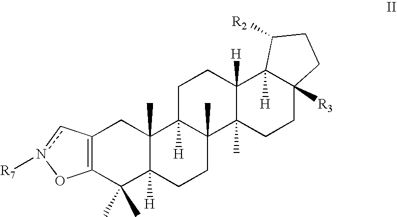
or a pharmaceutically acceptable salt thereof, wherein [symbol]
is a single or double bond, provided that when [symbol] is a double bond, R₇ is absent;
R₂ is C(CH₃)₂ or C(═CH₂)CH₃;
R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)R₄, CH₂OCO(CH₂)ₙX, CH₂NH₂, CH₂NHR₄, CH₂N(CH₂)ₙR₄R₅, CH₂NR₄R₅, CO₂R₄, C(O)NHR₄, or C(O)NR₄R₅;
R₄ and R₅ are independently H, C(O)X, halo, C₁₋₈ alkyl, aryl-C₁₋₈ alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;
R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;
R₇ is OH;
X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and
n=1-5.

5. A compound according to claim 4, wherein R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

6. A compound of the formula 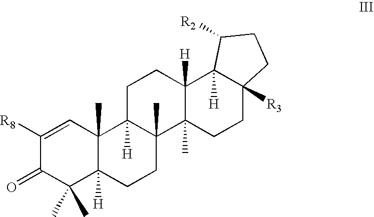
or a pharmaceutically acceptable salt thereof, wherein
R₂ is C(CH₃)₂ or C(═CH₂)CH₃;
R₃ is H, halo, CHO, CH₂OH, CH₂X, CH₂OR₄, CH₂OSi(R₄)ₙ(R₅)₃₋ₙ, CH₂OCOR₄, CH₂OC(O)OR₄, CH₂OC(O)NR₄R₅, CH₂OCO(HC═CH)R₄, CH₂OCO(CH₂)ₙX, CH₂OCOR₄, CH₂NHR₄, CH₂N(CH₂)ₙR₄R₅, CH₂NR₄R₅, CO₂R₄, C(O)NHR₄, or C(O)NR₄R₅,
R₄ and R₅ are independently H, C(O)X, halo, C₁₋₈ alkyl, aryl-C, g alkyl, cyclo(C₃₋₉)alkyl, (C₃₋₉) carbocycle, aryl, or heterocycle, wherein the alkyl is a straight or branched hydrocarbon; the carbocycle is saturated or unsaturated cyclic ring, the aryl is six membered aromatic carbocycle or a polycyclic aromatic hydrocarbon selected from phenyl, naphthyl, phenanthracenyl, indanyl; the heterocyle is six membered aromatic heterocycles, five membered aromatic heterocyles, 3 to 9 membered non-aromatic heterocycles or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein each of the alkyl, carbocycle, aryl or heterocycle is unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, OCOCH₃, carboxylic ester, carboxylic amide, sulfonic acid, sulfonic amide, CN, N₃, NHC(O)OC₁₋₈ alkyl, NHOH, ═NOH, NH₂, NO₂, OH, SH, F, Cl, Br, or I;
R₄ and R₅ may be combined to form a 3-9 membered saturated or unsaturated carbocycle, an aryl or a heterocycle, wherein the aryl is any six membered aromatic carbocycle or a polycyclic aromatic hydrocarbon selected from phenyl, naphthyl, phenanthracenyl, or indanyl; the heterocyle is five membered aromatic heterocyles, six membered aromatic heterocycles, 3 to 9 membered non-aromatic heterocycles, or bycyclic systems selected from piridyl, diazinyl, pyrimidinyl, 5-methoxy pyrimidinyl, pyrrolidinyl, (1,2,4)triazine-3,5-dione-6-yl, 6-mercaptopyrimidine-4yl, pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thiophenyl, piperazinyl, 4-methyl piperazinyl, pyranyl, morpholinyl, indolyl, benzthiopheneyl, benzofuranyl, isoindolyl, isobenzothiophenyl, isobenzofuranyl; wherein the alkyl, carbocycle, aryl or heterocycle may each be unsubstituted or substituted with one or more of the following: C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, carboxylic acid, carboxylic ester, carboxylic amide, sulfonic acid, sulfonic amide, CN, N₃, NHOH, ═NOH, NH₂, NO₂, OH, SH, F, Cl, Br, or I;
R₈ is H, CN, halo, Se-phenyl, OC₁₋₈ alkyl or C(O)H, or R₈ together with the ring to which it is attached form 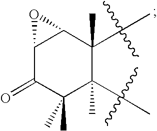
X is F, Cl, Br, I, CN, N₃, NHOH, ═NOH, NH₂, OH, SH, NHR₄, NR₄R₅, OR₄, SR₄, CO₂H, CO₂R₄, SO₃H₂, or SO₃R₄; and
n=1-5;
provided that when R₂ is C(CH₃)₂ and R₃ is CO₂H, R₈ cannot be CN, Cl or CHO; and when R₂ is C(CH₃)₂ and R₃ is CO₂Me, R₈ cannot be CN, OMe or CHO.

7. A compound according to claim 6, wherein R₃ is CHO, CO₂H, CH₂O-pyranyl, CH₂OH, CH₂OCO(CH₂)OCH₂CH₃, CH₂OCO(CH₂)Br, CH₂OCO(HC═CH)COOH, CH₂OCH₂CH₃, CH₂OCOCH₂OCH₃, CH₂NHCH₂C(O)OCH₃, CH₂NHCH₂C(O)OH, CH₂NHCH₂CH₂OH, or CH₂NHCH₂CH₂Cl, CH₂OCO(HC═CH)phenyl wherein the phenyl is substituted with two OCOCH₃ or OH.

8. A compound according to claim 6, wherein, R₈ is H, CN, CHO, Cl or OCH₃.

9. A pharmaceutical compositions comprising a compound according to claim 1 and pharmaceutically acceptable carrier, excipient, or diluent.

10. A pharmaceutical compositions comprising a compound according to claim 4 and pharmaceutically acceptable carrier, excipient, or diluent.

11. A pharmaceutical compositions comprising a compound according to claim 6 and pharmaceutically acceptable carrier, excipient, or diluent.

12. A method for inhibiting cancer in a cell comprising contacting the cell in which inhibition is desired with an effective amount of a compound according to claim 1 or a pharmaceutical composition according to claim 8.

13. A method for inhibiting cancer in a cell comprising contacting the cell in which inhibition is desired with an effective amount of a compound according to claim 4 or a pharmaceutical composition according to claim 9.

14. A method for inhibiting cancer in a cell comprising contacting the cell in which inhibition is desired with an effective amount of a compound according to claim 6 or a pharmaceutical composition according to claim 10.

15. A method of treating a disease comprising administering to a patient a pharmaceutical composition according to any one of claims 9-11.

16. The method according to claim 15, wherein the disease involves a cell proliferative condition.

17. The method according to claim 16, wherein the cell proliferative condition is cancer.

18. The method according to claim 17, wherein the cancer is melanoma, glioblastoma, ovarian carcinoma, colon carcinoma, and breast carcinoma, or cervical cancer.

19. A method for inhibiting viruses, bacteria or malaria in a cell comprising contacting the cell in which inhibition is desired with an effective amount of a compound according to any one of claims 1, 4, or 6 or a pharmaceutical composition according to any one of claims 9-11.

20. A method for treating inflammation comprising administering to a patient a pharmaceutical composition according to any one of claims 9-11.
